SEQUENCE LISTING (27 sequences)

SEQ ID NO: 1 - DNA, 20 bp, Artificial Sequence
  Oligonucleotide
aggcagttgagacgaacatt

SEQ ID NO: 2 - DNA, 43 bp, Artificial Sequence
  Oligonucleotide
cctaagtctgaaatttatcacccgccatagtagacgtatcacc

SEQ ID NO: 3 - DNA, 63 bp, Artificial Sequence
  Oligonucleotide
cttgctacacgattcagacttaggaatgttcgacatgcgagggtccaataccgacgattacag

SEQ ID NO: 4 - DNA, 43 bp, Artificial Sequence
  Oligonucleotide
ggtgataaaacgtgtagcaagctgtaatcgacgggaagagcat

SEQ ID NO: 5 - DNA, 20 bp, Artificial Sequence
  Oligonucleotide
gcccatccactactatggcg

SEQ ID NO: 6 - DNA, 63 bp, Artificial Sequence
  Oligonucleotide
cctcgcatgactcaactgcctggtgatacgaggatgggcatgctcttcccgacggtattggac

SEQ ID NO: 7 - DNA, 32 bp, Artificial Sequence
  Oligonucleotide
gtctgaggcagttgagagatctcgaacattcc

SEQ ID NO: 8 - DNA, 61 bp, Artificial Sequence
  Oligonucleotide
taagtctgaagatccatttatcaccagctgctgcacgccatagtagacgtatcacctgtcc

SEQ ID NO: 9 - DNA, 93 bp, Artificial Sequence
  Oligonucleotide
agctacttgctacacgaggatcttcagacttaggaatgttcgagatcacatgcgaggactcggtccaataccgtactaacgattacagatcaa

SEQ ID NO: 10 - DNA, 32 bp, Artificial Sequence
  Oligonucleotide
atgcccatccggctcactactatggcgtgcag

SEQ ID NO: 11 - DNA, 93 bp, Artificial Sequence
  Oligonucleotide
cgagtcctcgcatgactcaactgcctcagacggacaggtgatacgagagccggatgggcatgctcttcccgtagagacggtattggacatgat

SEQ ID NO: 12 - DNA, 21 bp, Artificial Sequence
  Oligonucleotide
ttttttttttttttttttttt

SEQ ID NO: 13 - DNA, 21 bp, Artificial Sequence
  Oligonucleotide
atcgtacgatcatagatcaat

SEQ ID NO: 14 - DNA, 21 bp, Artificial Sequence
  Oligonucleotide
tacagtcgtattgcattccga

SEQ ID NO: 15 - DNA, 21 bp, Artificial Sequence
  Oligonucleotide
attctagacgttacttaacat

SEQ ID NO: 16 - DNA, 21 bp, Artificial Sequence
  Oligonucleotide
taactatagctacaagctttc

SEQ ID NO: 17 - DNA, 21 bp, Artificial Sequence
  Oligonucleotide
ccataccgccatttccaacta

SEQ ID NO: 18 - DNA, 21 bp, Artificial Sequence
  Oligonucleotide
aagcacatgcgatgtttaact

SEQ ID NO: 19 - DNA, 21 bp, Artificial Sequence
  Oligonucleotide
aacgcugggcguuaaucaatt

SEQ ID NO: 20 - DNA, 21 bp, Artificial Sequence
  Oligonucleotide
uugauuaacgcccagcguutt

SEQ ID NO: 21 - DNA, 21 bp, Artificial Sequence
  Oligonucleotide
acaugaagcagcacgacuutt

SEQ ID NO: 22 - DNA, 21 bp, Artificial Sequence
  Oligonucleotide
aagucgugcugcuucaugutt

SEQ ID NO: 23 - DNA, 21 bp, Artificial Sequence
  Oligonucleotide
aaaaaaaaaaaaaaaaaaaaa

SEQ ID NO: 24 - DNA, 20 bp, Artificial Sequence
  Oligonucleotide
tccaacaccccaacatcttc

SEQ ID NO: 25 - DNA, 20 bp, Artificial Sequence
  Oligonucleotide
gtctttccgtgctccaaaac

SEQ ID NO: 26 - DNA, 19 bp, Artificial Sequence
  Oligonucleotide
agacctgcgacacctgcgt

SEQ ID NO: 27 - DNA, 61 bp, Artificial Sequence
  Oligonucleotide
cagctggtgataaaacgtgtagcaagtagctttgatctgtaatcgactctacgggaagagc

(19) United States
(12) Patent Application Publication
(10) Pub. No.: US 2014/0212503 A1
(43) Pub. Date: Jul. 31, 2014

(54) DELIVERY SYSTEM

(71) Applicants: Hyukjin Lee, Cambridge, MA (US); Abigail Lytton-Jean, Cambrige, MA (US); Angela Inok Park, Cambridge, MA (US); Robert S. Langer, Newton, MA (US); Daniel G. Anderson, Sudburg, MA (US)

(72) Inventors: Hyukjin Lee, Cambridge, MA (US); Abigail Lytton-Jean, Cambrige, MA (US); Angela Inok Park, Cambridge, MA (US); Robert S. Langer, Newton, MA (US); Daniel G. Anderson, Sudburg, MA (US)

(21) Appl. No.: 14/005,707

(22) PCT Filed: Mar. 17, 2012
(86) PCT No.: PCT/US12/29571

§ 371 Date: Jan. 24, 2014

Related U.S. Application Data
(60) Provisional application No. 61/453,668, filed on Mar. 17, 2011.
(60) Provisional application No. 61/544,014, filed on Oct. 6, 2011.

(51) Int. Cl. A61K 9/14; A61K 47/22; A61K 31/713

(52) U.S. Cl. CPC A61K 9/146; A61K 31/713; A61K 47/22
USPC 424/499; 435/375; 514/44 A

(57) ABSTRACT

The present invention provides three-dimensional, nanoscale delivery systems, particularly well adapted for delivery of nucleic acids and/or nucleic acid associated entities.

### CROSS REFERENCE TO RELATED APPLICATIONS

This application claims priority to U.S. Provisional Application Ser. No. 61/453,668, filed Mar. 17, 2011; and U.S. Provisional Application Ser. No. 61/544,014 filed on Oct. 6, 2011, the disclosures of which are both hereby incorporated by reference.

### BACKGROUND

There is a need for improved systems for the delivery of pharmaceutical and/or detectable entities.

### SUMMARY

The present invention encompasses the recognition that desirable delivery systems can be prepared from oligonucleotides whose structure is selected or designed to achieve assembly of the oligonucleotides into three dimensional structures with nanoscale dimensions, analogous in some ways to artificial viruses. As discussed in the embodiments and examples below, the present invention is, in part, based on the discovery that a self-assembling three-dimensional delivery structure can be generated using a plurality of oligonucleotides. Without wishing to be bound by any particular theory, it is contemplated that these may be administered to a subject, to deliver a therapeutic moiety, a detectable moiety, or another payload of interest. Such self-assembling three-dimensional delivery structures used alone or in combination, may permit and/or facilitate treatment, diagnosis or prognosis of a subject.

In one aspect, the present invention provides a nanoscale delivery system configured to carry and deliver a payload moiety. In some embodiments, provided delivery systems comprise a plurality of oligonucleotides, each with a first portion that is complementary to a first portion of another oligonucleotide in the plurality. In some embodiments, the oligonucleotides are configured to form a self-assembling three-dimensional nanoparticle through hybridization. In some embodiments, a self-assembled three-dimensional nanoparticle is generated whose arms are substantially double stranded. In some embodiments, a self-assembled three-dimensional nanoparticle is generated in the shape of a polyhedron. In some embodiments, the three-dimensional polyhedron is self-assembled so that the faces are congruent regular polygons which are assembled in the same way around each vertex to form a “regular polyhedron”. In some embodiments, the three-dimensional polyhedron is self-assembled so that the faces are not congruent regular polygons or assembled in the same way around each vertex, thereby forming an “irregular polyhedron”. In some embodiments, the three-dimensional polyhedron is in the shape of a tetrahedron. In some embodiments, the three-dimensional polyhedron is in the shape of a cube. In some embodiments, the three-dimensional polyhedron is in the shape of a hexahedron. In some embodiments, the three-dimensional polyhedron is in the shape of a heptahedron. In some embodiments, the three-dimensional polyhedron is in the shape of a octahedron. In some embodiments, the three-dimensional polyhedron is in the shape of a dodecahedron. In some embodiments, the three-dimensional polyhedron is in the shape of a icosahedron. In some embodiments, the three-dimensional polyhedron is in the shape of a icosidodecahedron. In some embodiments, the three-dimensional polyhedron is in the shape of a rhombic triacontahedron. In some embodiments, the self-assembling three-dimensional structure forms the shape of a stellation polyhedron. In some embodiments, the three-dimensional stellation polyhedron is in the shape of a any of the structures described in FIG. 27.

In some embodiments, the nanoscale delivery system comprises more than one self-assembled three-dimensional nanoparticle In some embodiments, the delivery system comprises at least 2, 3, 4, 5, 6, 7, 8, 9, 10, 11, or 12 self-assembling three-dimensional nanoparticles linked together. In some embodiments the at least 2, 3, 4, 5, 6, 7, 8, 9, 10, 11, or 12 self-assembling three-dimensional nanoparticles are linked together using a cleavage step. In some embodiments the at least 2, 3, 4, 5, 6, 7, 8, 9, 10, 11, or 12 self-assembling three-dimensional nanoparticles are linked together without using a cleavage step. In some embodiments the at least 2, 3, 4, 5, 6, 7, 8, 9, 10, 11, or 12 self-assembling three-dimensional nanoparticles are linked together without cutting the self assembled three-dimensional structure.

In some embodiments, the delivery system comprises at least 2, 3, 4, 5, 6, 7, 8, 9, 10, 11, or 12 self-assembling three-dimensional nanoparticles linked together through hybridization. In some embodiments, at least 2, 3, 4, 5, 6, 7, 8, 9, 10, 11, or 12 self-assembling three-dimensional nanoparticles are attached to each other by at least one common face. In some embodiments, at least 2, 3, 4, 5, 6, 7, 8, 9, 10, 11, or 12 self-assembling three-dimensional nanoparticles are attached to each other by a linking group. In some embodiments, the linking group is a chemical linking group. In some embodiments, the chemical linking group is an chemical linker as described in the application. In some embodiments, the linking group is a biological linking group. In some embodiments, the biological linking group is selected from the group consisting of DNA, RNA, protein, lipid, glycoprotein, glycolipid, carbohydrate and combinations thereof. In some embodiments, the biological linking group is part of the self-assembling three-dimensional nanoparticle (i.e. a hybridization element). In some embodiments, the biological linking group is not part of the self-assembling three-dimensional nanoparticle and is a separate molecule acting as a biological bridge. In some embodiments, the biological linking group contains a cleave site, wherein the cleavage site is cleavable by an enzyme selected from the group consisting of a protease, RNase, DNase, endonuclease, exonuclease, peptidase, glycosylase, restriction enzyme, lipase and phosphatase. In some embodiments, at least 2, 3, 4, 5, 6, 7, 8, 9, 10, 11, or 12 self-assembling three-dimensional nanoparticles have ligand hybridization elements with sequences selected to be complementary to a common “bridge” oligonucleotide, such that addition of the bridge oligonucleotide permits higher-order association of nanoparticles. In some embodiments, such a bridge oligonucleotide is or comprises a ligand oligonucleotide and/or a payload moiety (e.g., an siRNA, such as is illustrated, for example, in FIG. 17). In some embodiments, the bridge oligonucleotide contains a cleave site, wherein the cleavage site is cleavable by an enzyme selected from the group consisting of a protease, RNase, DNase, endonuclease, exonuclease, peptidase, glycosylase, restriction enzyme, lipase and phosphatase.

In some embodiments, at least some of the oligonucleotides within the plurality of oligonucleotides further comprise a second portion. In some embodiments the second portion is a ligand hybridization element. In some embodiments, the second portion does not hybridize with any other oligonucleotide in the plurality of oligonucleotides. In some embodiments, upon self-assembly of the three-dimensional nanoparticle, the three-dimensional nanoparticle is generated in which the second portion is single stranded. In some embodiments, the plurality of oligonucleotides are configured, wherein upon formation of the self-assembling three-dimensional nanoparticles, the single stranded second portions are on the exterior surface of the three-dimensional nanoparticle. In some embodiments, the plurality of oligonucleotides are configured, wherein upon formation of the self-assembled three-dimensions nanoparticle, the single stranded second portions are oriented within the interior of the three-dimensional structure.

In some embodiments, the delivery system comprises a self-assembled three-dimensional nanoparticle with at least one ligand hybridization element. In some embodiments, the delivery system comprises a self-assembled three-dimensional nanoparticle with a plurality of ligand hybridization elements. In some embodiments, the delivery system comprises a self-assembled three-dimensional nanoparticle with at least 2, 3, 4, 5, 6, 7, 8, 9, 10, 11 or 12 ligand hybridization elements. In some embodiments, the ligand hybridization elements are configured, wherein upon formation of the self-assembling three-dimensional nanoparticles, the ligand hybridization elements are on the exterior surface of the three-dimensional nanoparticle. In some embodiments, the ligand hybridization elements are configured, wherein upon formation of the self-assembling three-dimensional nanoparticles, at least 2 ligand hybridization element are on the exterior surface of the three-dimensional nanoparticle and within the same face and/or plane of the three-dimensional nanoparticle. In some embodiments, the ligand hybridization elements are configured, wherein upon formation of the self-assembling three-dimensional nanoparticles, a plurality of least 2 ligand hybridization elements are on the exterior surface of the three-dimensional nanoparticle and within the same face and/or plane of the three-dimensional nanoparticle. In some embodiments, ligand hybridization elements are configured, wherein upon formation of the self-assembled three-dimensions nanoparticle, the ligand hybridization elements are oriented within the interior of the three-dimensional structure.

In some embodiments, the ligand hybridization element has a nucleotide length of at least 10 bases. In some embodiments, the ligand hybridization element has a nucleotide length of at least 15 bases. In some embodiments, the ligand hybridization element has a nucleotide length of at least 20 bases. In some embodiments, the ligand hybridization element has a nucleotide length of at least 25 bases. In some embodiments, the ligand hybridization element has a nucleotide length of at least 30 bases. In some embodiments, the hybridization element comprises a nucleotide sequence comprising a plurality of purine residues. In some embodiments, the hybridization element comprises a nucleotide sequence comprising a plurality of pyrimidine residues. In some embodiments, the hybridization element comprises a nucleotide sequence selected from the group consisting of polyA and PolyT. In some embodiments comprises at least one single stranded ligand hybridization sequence with a 5′ end. In some embodiments comprises at least one single stranded ligand hybridization sequence with a 3′ end. In some embodiments, the nanoparticle comprises a plurality of 5′ and/or 3′ single stranded ligand hybridization sequences.

In some embodiments, the plurality of oligonuclotides are nucleic acid. In some embodiments the nucleic acid is purified from an organism. In some embodiments, the nucleic acid is synthetic. In some embodiments, the nucleic acid is generated using non-naturally occurring nucleotide bases. In some embodiments, the nucleic acid comprises deoxyribonucleic acids. In some embodiments the nucleic acid comprises ribonucleic acid. In yet other embodiments, the plurality of oligonucleotides are a mixture of DNA and RNA.

In some embodiments, the delivery system comprises at least one hybridized entity. In some embodiments, the delivery system comprises a plurality of hybridized entities. In some embodiments, the hybridized entity comprises a ligand oligonucleotide, wherein the ligand oligonucleotide comprises a ligand sequence substantially complementary to at least one ligand hybridization element in the nanoparticle and at least one payload moiety. In some embodiments, the delivery system comprises a plurality of ligand oligonucleotides. In some embodiments, the delivery system comprises at least 1, 2, 3, 4, 5, 6, 7, 8, 9, 10, 11, or 12 ligand oligonucleotides. In some embodiments, the ligand oligonucleotide is a nucleic acid. In some embodiments, the ligand oligonucleotide is DNA. In some embodiments, the ligand oligonucleotide is RNA.

In some embodiments, at least one payload moiety is covalently attached to the ligand oligonucleotide. In some embodiments a plurality of payload moieties is covalently attached to the ligand oligonucleotide. In some embodiments, at least 1, 2, 3, 4, 5, 6, 7, 8, 9, 10, 11, or 12 payload moieties are covalently attached to the ligand oligonucleotide. In some embodiments, at least one payload moiety is releasably attached to the ligand oligonucleotide. In some embodiments a plurality of payload moieties is releasably attached to the ligand oligonucleotide. In some embodiments, at least 1, 2, 3, 4, 5, 6, 7, 8, 9, 10, 11, or 12 payload moieties are releasably attached to the ligand oligonucleotide. In some embodiments, a plurality of different payload moieties are releasably and/or covalently attached to the ligand oligonuclotide. In some embodiments the payload moiety is selected from the group consisting of a therapeutic payload, detectable payload, targeting payload, delivery payload or combinations thereof.

In some embodiments, the payload moiety comprises an oligonucleotide. In some embodiments, the oligonucleotide is DNA. In some embodiments, the DNA comprises at least one non-natural residue. In some embodiments, the oligonucleotide is RNA. In some embodiments, the RNA is selected from the group consisting of siRNA (small interfering RNA), shRNA (small hairpin RNA), mRNA (messenger RNA), miRNA (micro RNA) and combinations thereof. In some embodiments, the RNA has enzymatic activity (i.e. ribozyme).

In some embodiments, the hybridized entity comprises a cleavage site. In some embodiments, the hybridized entity is a ligand oligonucleotide, wherein the ligand oligonucleotide comprises a cleavage site. In some embodiments, the cleavage site is located within the ligand sequence. In some embodiments the cleavage site is located between the ligand oligonucleotide and the payload moiety. In some embodiments, the cleavage site is cleavable by an enzyme selected from the group consisting of a protease, RNase, DNase, endonuclease, exonuclease, peptidase, glycosylase, restriction enzyme, lipase and phosphatase.

In one aspect, the present invention provides a nanoscale delivery system configured to carry and deliver a payload moiety. In some embodiments, the delivery system comprise a plurality of oligonucleotides, each with a first portion that is complementary to a first portion of another oligonucleotide in the plurality, such that the plurality of oligonucleotides self-assembles to form a nanoparticle with a three-dimensional tetrahedral structure comprising a plurality of faces defined by substantially double stranded arms, wherein at least some oligonucleotides within the plurality have a structure that further comprises; a second portion that does not hybridized with another oligonucleotide in the plurality, so that such second portions is available for hybridizing to a payload moiety.

In some embodiment, the ligand hybridization element comprises a single stranded portion. In some embodiments, the ligand hybridization element comprises a single stranded overhang. In some embodiments, the single stranded overhand is a 3′ overhang. In some embodiments the single stranded overhang is a 5′ overhang.

In some embodiments, the ligand hybridization element has a nucleotide length of at least 10 bases. In some embodiments, the ligand hybridization element has a nucleotide length of at least 15 bases. In some embodiments, the ligand hybridization element has a nucleotide length of at least 20 bases. In some embodiments, the ligand hybridization element has a nucleotide length of at least 25 bases. In some embodiments, the ligand hybridization element has a nucleotide length of at least 30 bases. In some embodiments, the hybridization element comprises a nucleotide sequence comprising a plurality of purine residues. In some embodiments, the hybridization element comprises a nucleotide sequence comprising a plurality of pyrimidine residues. In some embodiments, the hybridization element comprises a nucleotide sequence selected from the group consisting of polyA and PolyT.

In some embodiments, the nanoparticle comprises at least 1 ligand hybridization element. In some embodiments, the nanoparticle comprises a plurality of ligand hybridization elements. In some embodiments, the nanoparticle comprises at least 1, 2, 3, 4, 5, 6, 7, 8, 9, 10, 11 or 12 ligand hybridization elements.

In some embodiments, nanoparticle comprises at least one payload moiety. In some embodiments, the nanoparticle comprises a plurality of payload moieties. In some embodiments, the nanoparticle comprise at least 1, 2, 3, 4, 5, 6, 7, 8, 9, 10, 11, or 12 payload moieties. In some embodiments, the payload moiety is a nucleic acid. In some embodiments, the nucleic acid is DNA. In some embodiments, the nucleic acid is RNA. In some embodiments, the RNA is selected from the group consisting of siRNA, shRNA, miRNA, mRNA, Ribozyme or combinations thereof. In some embodiments, the payload is siRNA.

In some embodiments, the nanoparticle comprises an siRNA payload. In some embodiments, the nanoparticle comprises at least one siRNA payload. In some embodiments, the nanoparticle comprises a plurality of siRNA payloads. In some embodiments, the nanoparticle comprises at least 1, 2, 3, 4, 5, 6, 7, 8, 9, 10, 11 or 12 siRNA payloads. In some embodiments, the siRNA payload is single stranded. In some embodiments, the siRNA payload is double stranded. In some embodiments the siRNA payload is a duplex. In some embodiments, the siRNA payload comprises a single stranded region and a double stranded regions. In some embodiments, the siRNA payload comprises a 3′ overhang. In some embodiments, the siRNA payload has a 5′ overhang. In some embodiments, the siRNa has a 3′ and a 5′ overhang.

In some embodiments, the siRNA payload hybridizes to the nanoparticle. In some embodiments, the siRNA payload hybridizes to the nanoparticle by way of a 3′ overhang. In some embodiments, the siRNA payload hybridizes to the nanoparticle by way of a 5′ overhang. In some embodiments, the siRNA payload hybridizes to the nanoparticle by way of a 3′ overhang. In some embodiments, the siRNA payload hybridizes to the nanoparticle by way of hybridizing between its 3′ overhang and one of the nucleotide's ligand hybridization elements, so that the 3′ overhang is the ligand hybridization element's complementary ligand. In some embodiments, the siRNA payload is attached to the nanoparticle using a chemical linker. In some embodiments, the siRNA payload is attached to the nanoparticle using a biological linker. In some embodiments, the siRNA payload is attached using a bridge.

In some embodiments, the delivery system comprises at least one siRNA payload. In some embodiments, the delivery system comprises a plurality of siRNA payloads. In some embodiments, the delivery system comprise at least 1, 2, 3, 4, 5, 6, 7, 8, 9, 10, 11, or 12 siRNA payloads.

In some embodiments, the delivery system comprises at least one targeting payload. In some embodiments, the delivery system comprises a plurality of targeting payloads. In some embodiments, the delivery system comprise at least 1, 2, 3, 4, 5, 6, 7, 8, 9, 10, 11, or 12 targeting payloads. In some embodiments the oligonucleotides within the plurality being designed and constructed such that, when the at least three targeting payload moieties hybridize to the ligand hybridization elements, the at least three targeting payload moieties are displayed on the same face of the nanoparticle.

In some embodiments, the delivery system comprise a plurality of targeting payloads and siRNA payloads. In some embodiments, the number of targeting payloads is empirically determined so that it does no saturate the total number of receptors on the cell surface. In some embodiments, the number of targeting payload is less than the total number of other payloads. In some embodiments, the number of targeting payloads is more than the total number of other payloads.

In one aspect, the present invention provides a method for generating a nanoparticle delivery system comprising, providing a plurality of oligonucleotides, each of which has a structure comprising a first portion that is complementary to a first portion of another oligonuclotide in the plurality such that the plurality of oligonucleotides self-assembles to form a three-dimensional nanoparticle whose arms are substantially double stranded, wherein at least some oligonucleotides in the plurality have a structure which further comprise a second portion comprising a ligand hybridization element; combining the plurality of oligonucleotides in a reaction buffer to form a reaction mixture; and maintaining the combination for a time and under conditions sufficient to permit self-assembly of the nanoparticle. In some embodiments, the step of maintaining further comprise, subjecting the reaction mixture to a heat treatment to denature the plurality of oligonuclotides; and cooling the reaction mixture to anneal the plurality of oligonucleotides to promote self-assembly of the three-dimensional nanoparticle. In some embodiments, the denaturation step is performed at a temperature which is at least 95° C. or greater.

In some embodiments of the method, the oligonucleotides are configured to form a self-assembling three-dimensional nanoparticle through hybridization. In some embodiments, a self-assembled three-dimensional nanoparticle is generated whose arms are substantially double stranded. In some embodiments, a self-assembled three-dimensional nanoparticle is generated in the shape of a polyhedron. In some embodiments, the three-dimensional polyhedron is self-assembled so that the faces are congruent regular polygons which are assembled in the same way around each vertex to form a “regular polyhedron”. In some embodiments, the three-dimensional polyhedron is self-assembled so that the faces are not congruent regular polygons or assembled in the same way around each vertex, thereby forming an “irregular polyhedron”. In some embodiments, the three-dimensional polyhedron is in the shape of a tetrahedron. In some embodiments, the three-dimensional polyhedron is in the shape of a cube. In some embodiments, the three-dimensional polyhedron is in the shape of a hexahedron. In some embodiments, the three-dimensional polyhedron is in the shape of a heptahedron. In some embodiments, the three-dimensional polyhedron is in the shape of a octahedron. In some embodiments, the three-dimensional polyhedron is in the shape of a dodecahedron. In some embodiments, the three-dimensional polyhedron is in the shape of a icosahedron. In some embodiments, the three-dimensional polyhedron is in the shape of a icosidodecahedron. In some embodiments, the three-dimensional polyhedron is in the shape of a rhombic triacontahedron. In some embodiments, the self-assembling three-dimensional structure forms the shape of a stellation polyhedron. In some embodiments, the three-dimensional stellation polyhedron is in the shape of a any of the structures described in FIG. 27.

In some embodiments of the method, the nanoparticle delivery system comprises more than one self-assembled three-dimensional nanoparticles. In some embodiments, the delivery system comprises at least 2, 3, 4, 5, 6, 7, 8, 9, 10, 11, or 12 self-assembling three-dimensional nanoparticles linked together. In some embodiments the at least 2, 3, 4, 5, 6, 7, 8, 9, 10, 11, or 12 self-assembling three-dimensional nanoparticles are linked together using a cleavage step. In some embodiments the at least 2, 3, 4, 5, 6, 7, 8, 9, 10, 11, or 12 self-assembling three-dimensional nanoparticles are linked together without using a cleavage step. In some embodiments the at least 2, 3, 4, 5, 6, 7, 8, 9, 10, 11, or 12 self-assembling three-dimensional nanoparticles are linked together without cutting the self assembled three-dimensional structure.

In some embodiments of the method, the nanoscale delivery system comprises at least 2, 3, 4, 5, 6, 7, 8, 9, 10, 11, or 12 self-assembling three-dimensional nanoparticles linked together through hybridization. In some embodiments, at least 2, 3, 4, 5, 6, 7, 8, 9, 10, 11, or 12 self-assembling three-dimensional nanoparticles are attached to each other by at least one common face. In some embodiments, at least 2, 3, 4, 5, 6, 7, 8, 9, 10, 11, or 12 self-assembling three-dimensional nanoparticles are attached to each other by a linking group. In some embodiments, the linking group is a chemical linking group. In some embodiments, the chemical linking group is an chemical linker as described in the application. In some embodiments, the linking group is a biological linking group. In some embodiments, the biological linking group is selected from the group consisting of DNA, RNA, protein, lipid, glycoprotein, glycolipid, carbohydrate and combinations thereof. In some embodiments, the biological linking group is part of the self-assembling three-dimensional nanoparticle (i.e. a hybridization element). In some embodiments, the biological linking group is not part of the self-assembling three-dimensional nanoparticle and is a separate molecule acting as a biological bridge. In some embodiments, the biological linking group contains a cleave site, wherein the cleavage site is cleavable by an enzyme selected from the group consisting of a protease, RNase, DNase, endonuclease, exonuclease, peptidase, glycosylase, restriction enzyme, lipase and phosphatase. In some embodiments, at least 2, 3, 4, 5, 6, 7, 8, 9, 10, 11, or 12 self-assembling three-dimensional nanoparticles have ligand hybridization elements with sequences selected to be complementary to a common “bridge” oligonucleotide, such that addition of the bridge oligonucleotide permits higher-order association of nanoparticles. In some embodiments, such a bridge oligonucleotide is or comprises a ligand oligonucleotide and/or a payload moiety (e.g., an siRNA, such as is illustrated, for example, in FIG. 17). In some embodiments, the bridge oligonucleotide contains a cleave site, wherein the cleavage site is cleavable by an enzyme selected from the group consisting of a protease, RNase, DNase, endonuclease, exonuclease, peptidase, glycosylase, restriction enzyme, lipase and phosphatase.

In some embodiments of the method, the second portion is a ligand hybridization element. In some embodiments, the second portion does not hybridize with any other oligonucleotide in the plurality of oligonucleotides. In some embodiments, upon self-assembly of the three-dimensional nanoparticle, the three-dimensional nanoparticle is generated in which the second portion is single stranded. In some embodiments, the plurality of oligonucleotides are configured, wherein upon formation of the self-assembling three-dimensional nanoparticles, the single stranded second portions are on the exterior surface of the three-dimensional nanoparticle. In some embodiments, the plurality of oligonucleotides are configured, wherein upon formation of the self-assembled three-dimensions nanoparticle, the single stranded second portions are oriented within the interior of the three-dimensional structure.

In some embodiments, the delivery system comprises a self-assembled three-dimensional nanoparticle with at least one ligand hybridization element. In some embodiments, the delivery system comprises a self-assembled three-dimensional nanoparticle with a plurality of ligand hybridization elements. In some embodiments, the delivery system comprises a self-assembled three-dimensional nanoparticle with at least 2, 3, 4, 5, 6, 7, 8, 9, 10, 11 or 12 ligand hybridization elements. In some embodiments, the ligand hybridization elements are configured, wherein upon formation of the self-assembling three-dimensional nanoparticles, the ligand hybridization elements are on the exterior surface of the three-dimensional nanoparticle. In some embodiments, the ligand hybridization elements are configured, wherein upon formation of the self-assembling three-dimensional nanoparticles, at least 2 ligand hybridization element are on the exterior surface of the three-dimensional nanoparticle and within the same face and/or plane of the three-dimensional nanoparticle. In some embodiments, the ligand hybridization elements are configured, wherein upon formation of the self-assembling three-dimensional nanoparticles, a plurality of least 2 ligand hybridization elements are on the exterior surface of the three-dimensional nanoparticle and within the same face and/or plane of the three-dimensional nanoparticle. In some embodiments, ligand hybridization elements are configured, wherein upon formation of the self-assembled three-dimensions nanoparticle, the ligand hybridization elements are oriented within the interior of the three-dimensional structure.

In some embodiments, the ligand hybridization element has a nucleotide length of at least 10 bases. In some embodiments, the ligand hybridization element has a nucleotide length of at least 15 bases. In some embodiments, the ligand hybridization element has a nucleotide length of at least 20 bases. In some embodiments, the ligand hybridization element has a nucleotide length of at least 25 bases. In some embodiments, the ligand hybridization element has a nucleotide length of at least 30 bases. In some embodiments, the hybridization element comprises a nucleotide sequence comprising a plurality of purine residues. In some embodiments, the hybridization element comprises a nucleotide sequence comprising a plurality of pyrimidine residues. In some embodiments, the hybridization element comprises a nucleotide sequence selected from the group consisting of polyA and PolyT. In some embodiments comprises at least one single stranded ligand hybridization sequence with a 5′ end. In some embodiments comprises at least one single stranded ligand hybridization sequence with a 3′ end. In some embodiments, the nanoparticle comprises a plurality of 5′ and/or 3′ single stranded ligand hybridization sequences.

In some embodiments, the plurality of oligonuclotides are nucleic acid. In some embodiments the nucleic acid is purified from an organism. In some embodiments, the nucleic acid is synthetic. In some embodiments, the nucleic acid is generated using non-naturally occurring nucleotide bases. In some embodiments, the nucleic acid comprises deoxyribonucleic acids. In some embodiments the nucleic acid comprises ribonucleic acid. In yet other embodiments, the plurality of oligonucleotides are a mixture of DNA and RNA.

In one aspect, the present invention provides a kit for creating a nanoscale delivery system configured to carry and delivery a payload moiety. In some embodiments, the kit comprises a plurality of oligonucleotides, each which has a structure comprising; a first portion that is complementary to a first portion of another oligonucleotide in the plurality, such that the plurality of oligonucleotides self-assembles to form the nanoparticle, which has a three-dimensional structure comprising a plurality of faces defined by substantially double stranded arms. In some embodiments, at least some of the oligonucleotides within the plurality of oligonucleotides have a structure that further comprises; a second portion that does not hybridize with another oligonucleotide in the plurality, so that such portions are single stranded ligand hybridization elements.

In some embodiments of the kit, the oligonucleotides are configured to form a self-assembling three-dimensional nanoparticle through hybridization. In some embodiments, a self-assembled three-dimensional nanoparticle is generated whose arms are substantially double stranded. In some embodiments, a self-assembled three-dimensional nanoparticle is generated in the shape of a polyhedron. In some embodiments, the three-dimensional polyhedron is self-assembled so that the faces are congruent regular polygons which are assembled in the same way around each vertex to form a “regular polyhedron”. In some embodiments, the three-dimensional polyhedron is self-assembled so that the faces are not congruent regular polygons or assembled in the same way around each vertex, thereby forming an “irregular polyhedron”. In some embodiments, the three-dimensional polyhedron is in the shape of a tetrahedron. In some embodiments, the three-dimensional polyhedron is in the shape of a cube. In some embodiments, the three-dimensional polyhedron is in the shape of a hexahedron. In some embodiments, the three-dimensional polyhedron is in the shape of a heptahedron. In some embodiments, the three-dimensional polyhedron is in the shape of a octahedron. In some embodiments, the three-dimensional polyhedron is in the shape of a dodecahedron. In some embodiments, the three-dimensional polyhedron is in the shape of a icosahedron. In some embodiments, the three-dimensional polyhedron is in the shape of a icosidodecahedron. In some embodiments, the three-dimensional polyhedron is in the shape of a rhombic triacontahedron. In some embodiments, the self-assembling three-dimensional structure forms the shape of a stellation polyhedron. In some embodiments, the three-dimensional stellation polyhedron is in the shape of a any of the structures described in FIG. 27.

In some embodiments of the kit, the nanoparticle delivery system comprises more than one self-assembled three-dimensional nanoparticles. In some embodiments, the delivery system comprises at least 2, 3, 4, 5, 6, 7, 8, 9, 10, 11, or 12 self-assembling three-dimensional nanoparticles linked together. In some embodiments the at least 2, 3, 4, 5, 6, 7, 8, 9, 10, 11, or 12 self-assembling three-dimensional nanoparticles are linked together using a cleavage step. In some embodiments the at least 2, 3, 4, 5, 6, 7, 8, 9, 10, 11, or 12 self-assembling three-dimensional nanoparticles are linked together without using a cleavage step. In some embodiments the at least 2, 3, 4, 5, 6, 7, 8, 9, 10, 11, or 12 self-assembling three-dimensional nanoparticles are linked together without cutting the self assembled three-dimensional structure.

In some embodiments of the kit, the nanoscale delivery system comprises at least 2, 3, 4, 5, 6, 7, 8, 9, 10, 11, or 12 self-assembling three-dimensional nanoparticles linked together through hybridization. In some embodiments, at least 2, 3, 4, 5, 6, 7, 8, 9, 10, 11, or 12 self-assembling three-dimensional nanoparticles are attached to each other by at least one common face. In some embodiments, at least 2, 3, 4, 5, 6, 7, 8, 9, 10, 11, or 12 self-assembling three-dimensional nanoparticles are attached to each other by a linking group. In some embodiments, the linking group is a chemical linking group. In some embodiments, the chemical linking group is an chemical linker as described in the application. In some embodiments, the linking group is a biological linking group. In some embodiments, the biological linking group is selected from the group consisting of DNA, RNA, protein, lipid, glycoprotein, glycolipid, carbohydrate and combinations thereof. In some embodiments, the biological linking group is part of the self-assembling three-dimensional nanoparticle (i.e. a hybridization element). In some embodiments, the biological linking group is not part of the self-assembling three-dimensional nanoparticle and is a separate molecule acting as a biological bridge. In some embodiments, the biological linking group contains a cleave site, wherein the cleavage site is cleavable by an enzyme selected from the group consisting of a protease, RNase, DNase, endonuclease, exonuclease, peptidase, glycosylase, restriction enzyme, lipase and phosphatase. In some embodiments, at least 2, 3, 4, 5, 6, 7, 8, 9, 10, 11, or 12 self-assembling three-dimensional nanoparticles have ligand hybridization elements with sequences selected to be complementary to a common “bridge” oligonucleotide, such that addition of the bridge oligonucleotide permits higher-order association of nanoparticles. In some embodiments, such a bridge oligonucleotide is or comprises a ligand oligonucleotide and/or a payload moiety (e.g., an siRNA, such as is illustrated, for example, in FIG. 17). In some embodiments, the bridge oligonucleotide contains a cleave site, wherein the cleavage site is cleavable by an enzyme selected from the group consisting of a protease, RNase, DNase, endonuclease, exonuclease, peptidase, glycosylase, restriction enzyme, lipase and phosphatase.

In some embodiments, upon self-assembly of the three-dimensional nanoparticle, the three-dimensional nanoparticle is generated in which the second portion is single stranded. In some embodiments, the plurality of oligonucleotides are configured, wherein upon formation of the self-assembling three-dimensional nanoparticles, the single stranded second portions are on the exterior surface of the three-dimensional nanoparticle. In some embodiments, the plurality of oligonucleotides are configured, wherein upon formation of the self-assembled three-dimensions nanoparticle, the single stranded second portions are oriented within the interior of the three-dimensional structure.

In some embodiments, the delivery system comprises a self-assembled three-dimensional nanoparticle with at least one ligand hybridization element. In some embodiments, the delivery system comprises a self-assembled three-dimensional nanoparticle with a plurality of ligand hybridization elements. In some embodiments, the delivery system comprises a self-assembled three-dimensional nanoparticle with at least 2, 3, 4, 5, 6, 7, 8, 9, 10, 11 or 12 ligand hybridization elements. In some embodiments, the ligand hybridization elements are configured, wherein upon formation of the self-assembling three-dimensional nanoparticles, the ligand hybridization elements are on the exterior surface of the three-dimensional nanoparticle. In some embodiments, the ligand hybridization elements are configured, wherein upon formation of the self-assembling three-dimensional nanoparticles, at least 2 ligand hybridization element are on the exterior surface of the three-dimensional nanoparticle and within the same face and/or plane of the three-dimensional nanoparticle. In some embodiments, the ligand hybridization elements are configured, wherein upon formation of the self-assembling three-dimensional nanoparticles, a plurality of least 2 ligand hybridization elements are on the exterior surface of the three-dimensional nanoparticle and within the same face and/or plane of the three-dimensional nanoparticle. In some embodiments, ligand hybridization elements are configured, wherein upon formation of the self-assembled three-dimensions nanoparticle, the ligand hybridization elements are oriented within the interior of the three-dimensional structure.

In some embodiments, the ligand hybridization element has a nucleotide length of at least 10 bases. In some embodiments, the ligand hybridization element has a nucleotide length of at least 15 bases. In some embodiments, the ligand hybridization element has a nucleotide length of at least 20 bases. In some embodiments, the ligand hybridization element has a nucleotide length of at least 25 bases. In some embodiments, the ligand hybridization element has a nucleotide length of at least 30 bases. In some embodiments, the hybridization element comprises a nucleotide sequence comprising a plurality of purine residues. In some embodiments, the hybridization element comprises a nucleotide sequence comprising a plurality of pyrimidine residues. In some embodiments, the hybridization element comprises a nucleotide sequence selected from the group consisting of polyA and PolyT. In some embodiments comprises at least one single stranded ligand hybridization sequence with a 5′ end. In some embodiments comprises at least one single stranded ligand hybridization sequence with a 3′ end. In some embodiments, the nanoparticle comprises a plurality of 5′ and/or 3′ single stranded ligand hybridization sequences.

In some embodiments, the plurality of oligonuclotides are nucleic acid. In some embodiments the nucleic acid is purified from an organism. In some embodiments, the nucleic acid is synthetic. In some embodiments, the nucleic acid is generated using non-naturally occurring nucleotide bases. In some embodiments, the nucleic acid comprises deoxyribonucleic acids. In some embodiments the nucleic acid comprises ribonucleic acid. In yet other embodiments, the plurality of oligonucleotides are a mixture of DNA and RNA.

### BRIEF DESCRIPTION OF DRAWING

FIG. 1: Illustrates the three dimensional geometry of a tetrahedral structure.

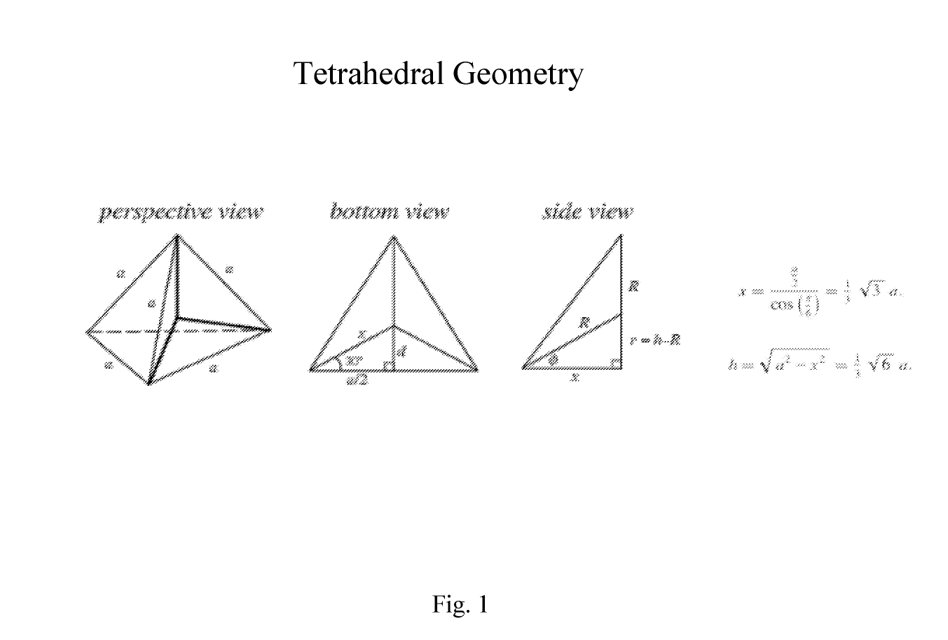

FIG. 2: Illustrates preparation of self-assembled DNA fragments into tetrahedral structures with sticky ends, available for ligand (e.g., siRNA) hybridization as described herein.

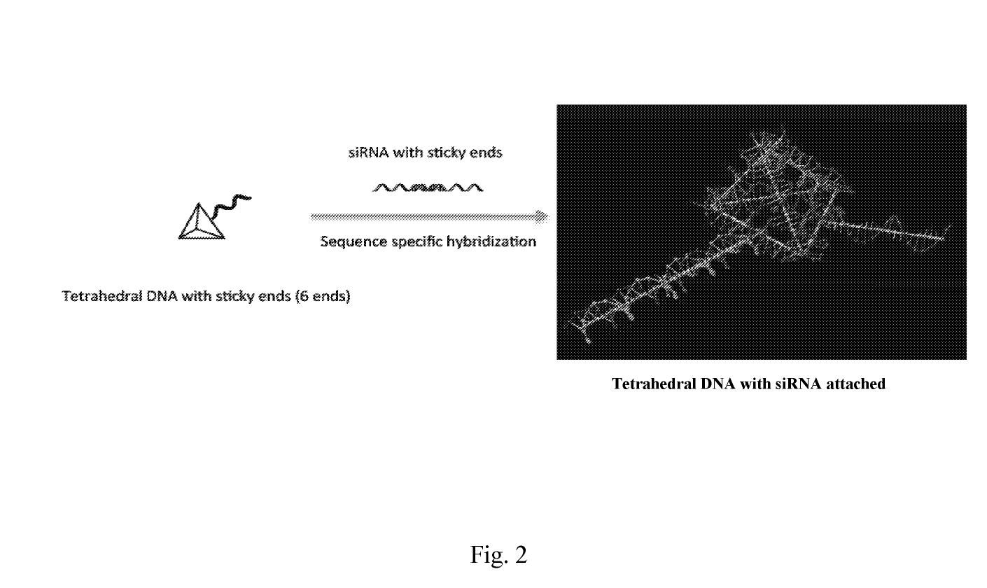

FIG. 3: Illustrates an example of a self-assembled DNA fragments into a tetrahedral structure with sticky ends, available for ligand, along with a description of certain benefits of the provided delivery structure.

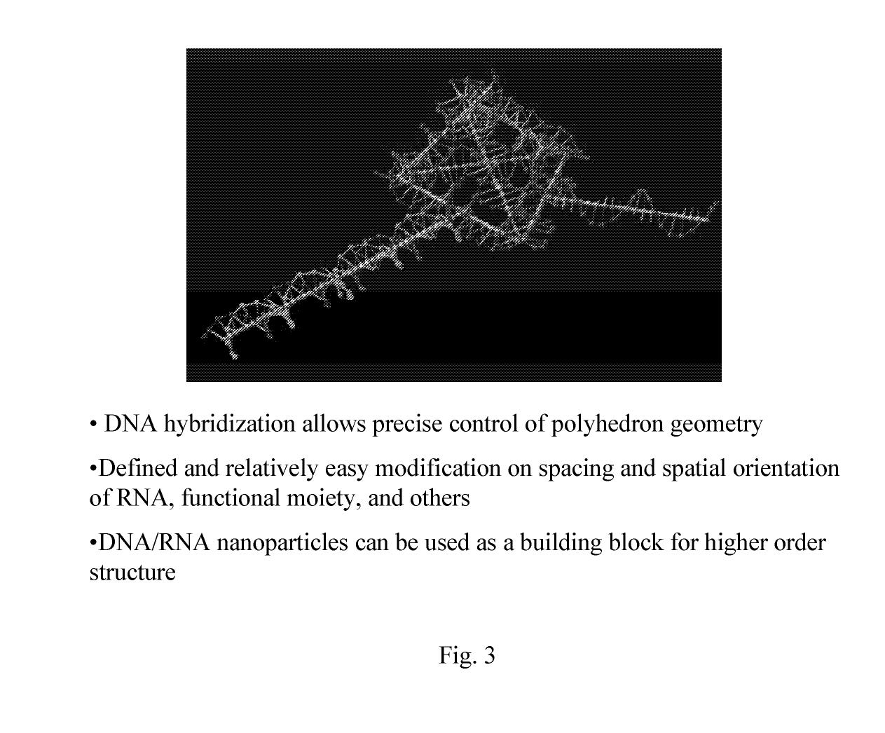

FIG. 4: Depicts preparation of a 6-nick tetrahedral delivery system according to the present invention from indicated oligonucleotides.

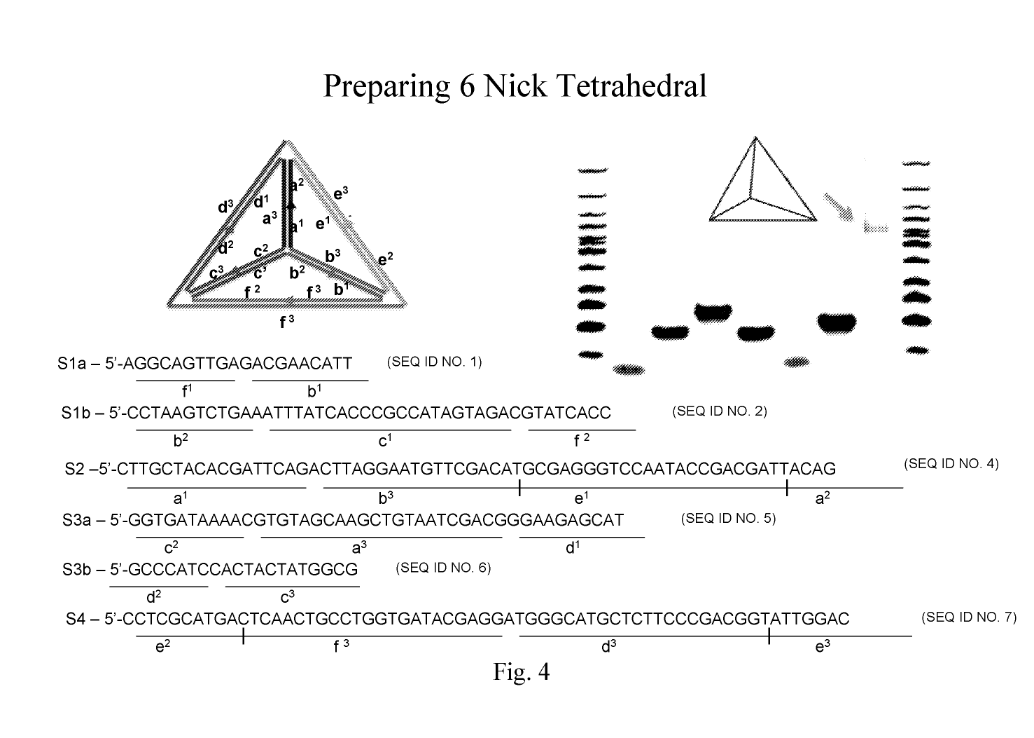

FIG. 5: Depicts a 6-nick tetrahedral delivery system associated with siRNAs.

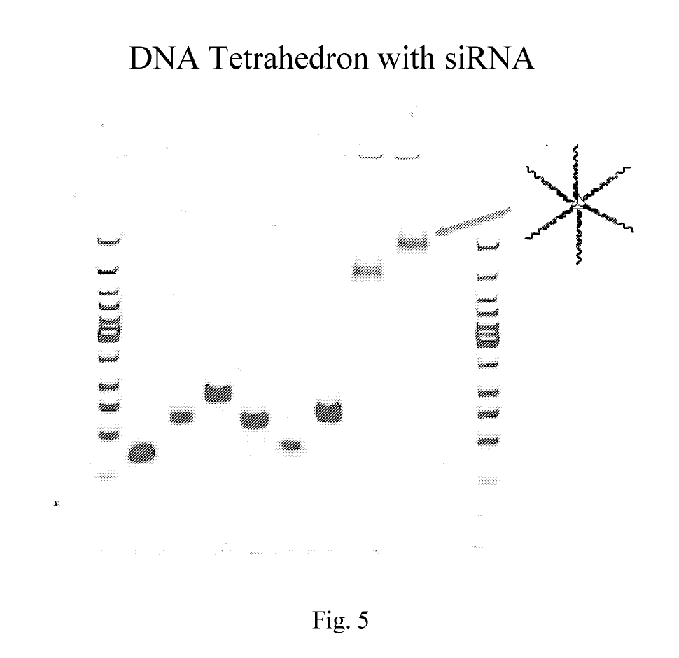

FIG. 6: Shows hydrodynamic radius vs % intensity (Rh˜15.8) for certain provided delivery structures

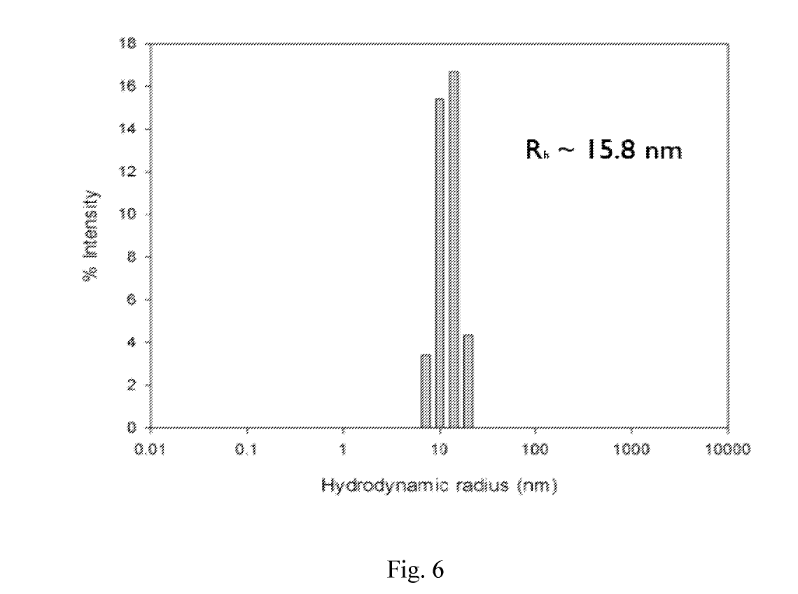

FIG. 7: Presents images of exemplary tetrahedral delivery systems provided herein, under different magnification.

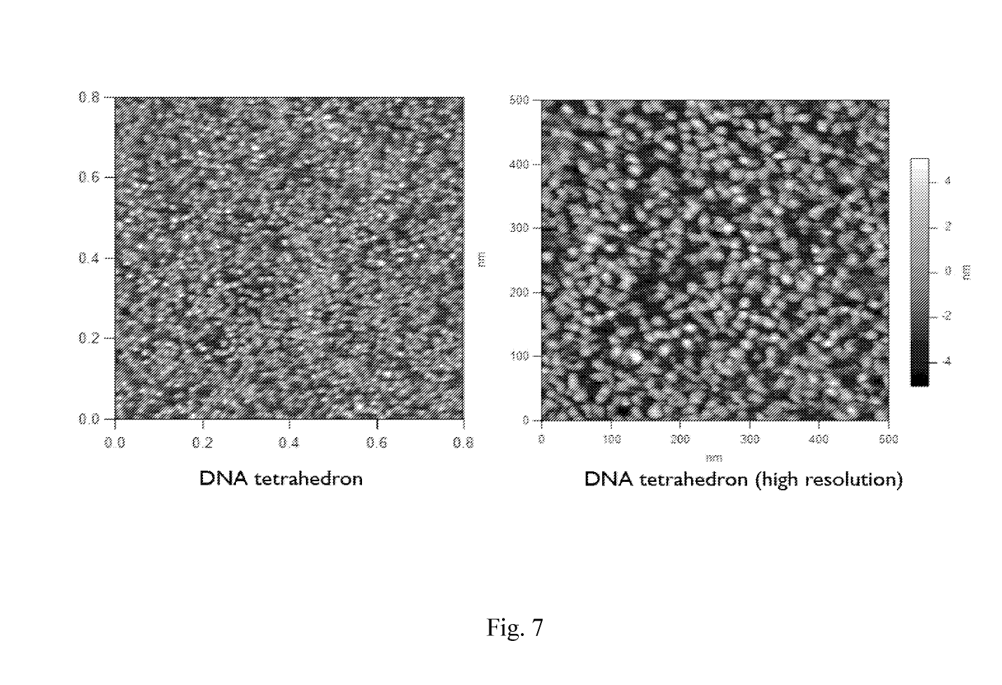

FIG. 8: Depicts association (e.g., conjugation) of exemplary cell-penetrating peptides, non-charged peptides from manos, Folate, or Polyethylene glycol 3400 to facilitate targeting and/or delivery of certain exemplary delivery systems in accordance with the present invention.

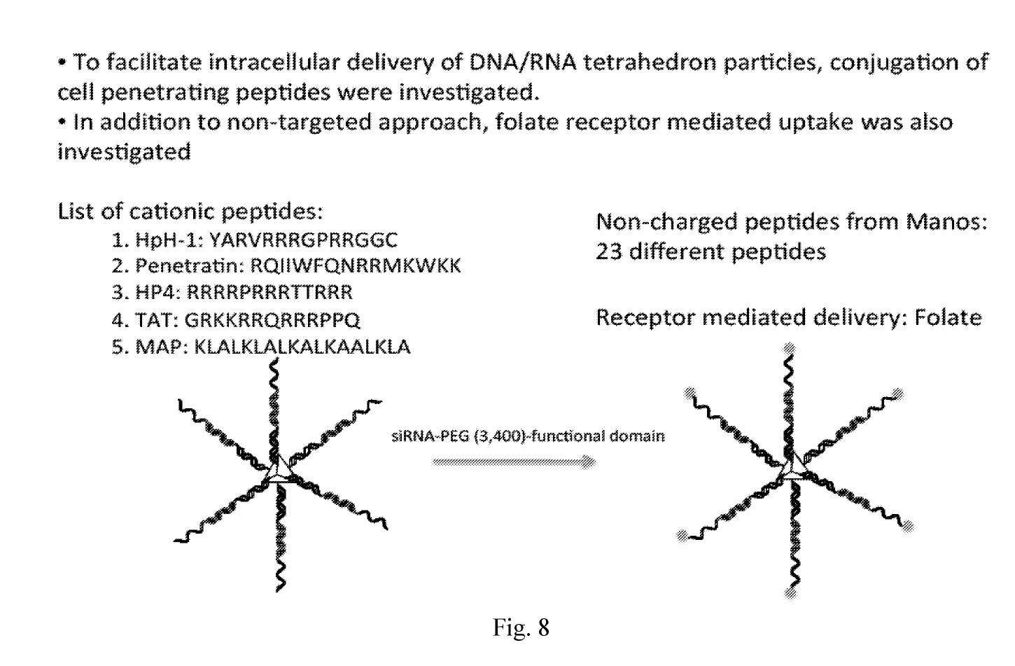

FIG. 9: Depicts strategies for association/coupling of an exemplary DNA tetrahedron delivery system with various entities such as, for example, polyT, siRNA, PEG, etc

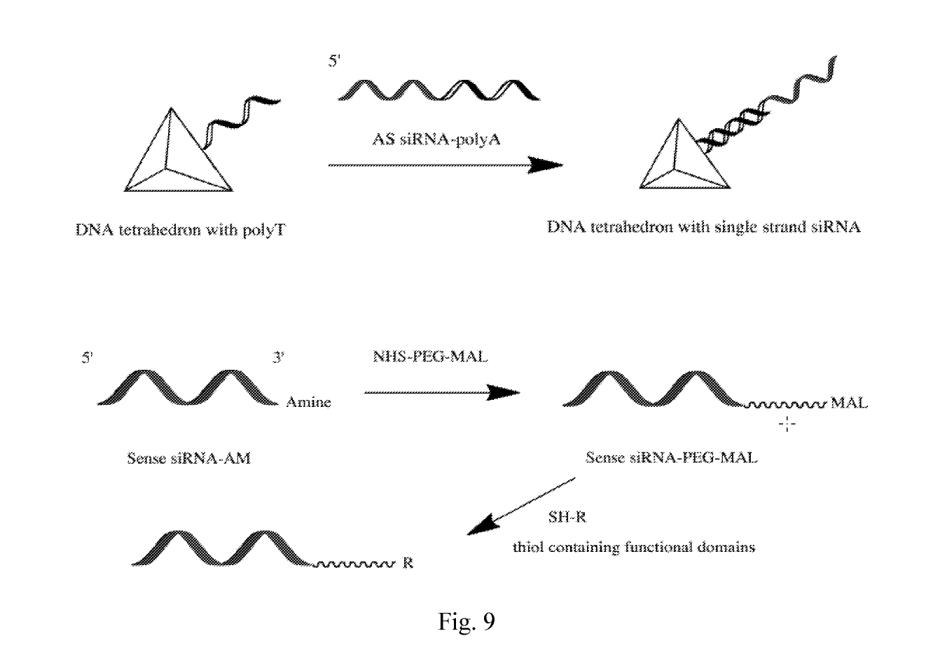

FIG. 10: Depicts an exemplary DNA tetrahedron delivery systems with ds-siRNA-PEG-functional domains.

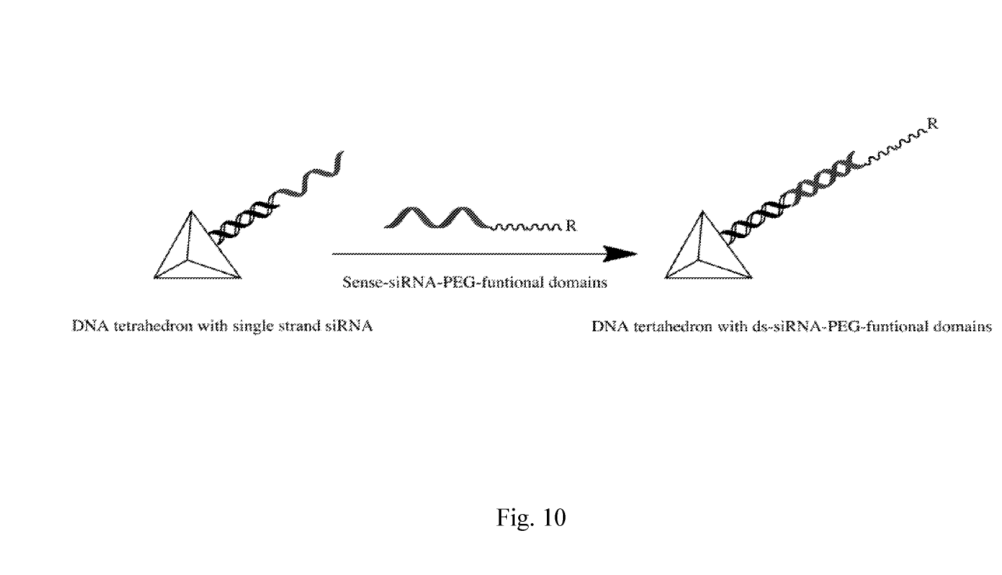

FIG. 11: Highlights certain cell penetrating/targeting domains and their ability to delivery an agent that reduces luciferase expression (%).

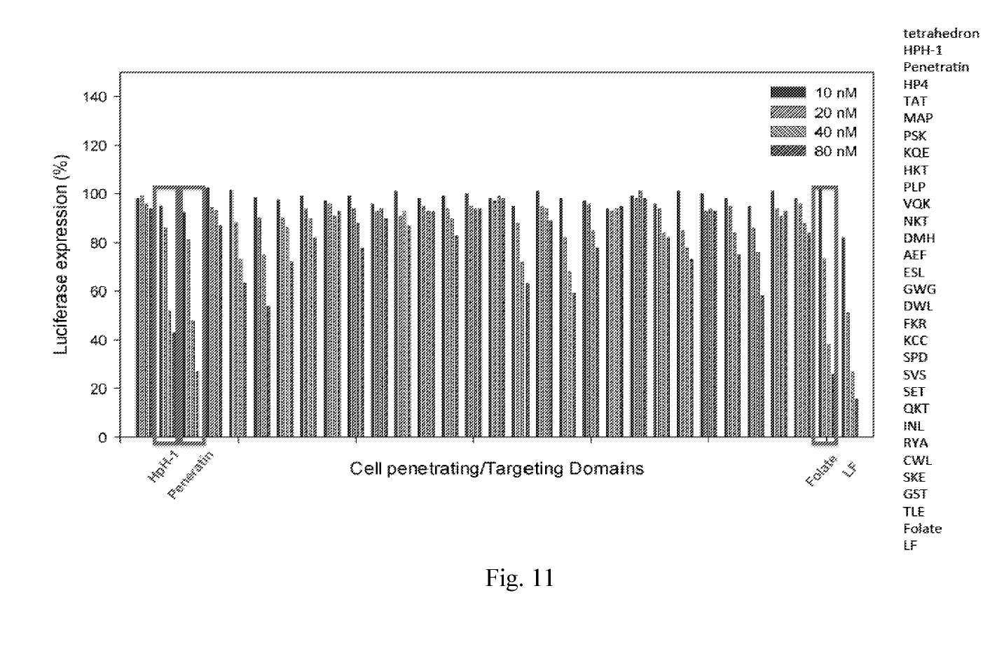

FIG. 12: Depicts GFP expression levels of various delivery systems with and without a Folate targeting agent in accordance with the present invention.

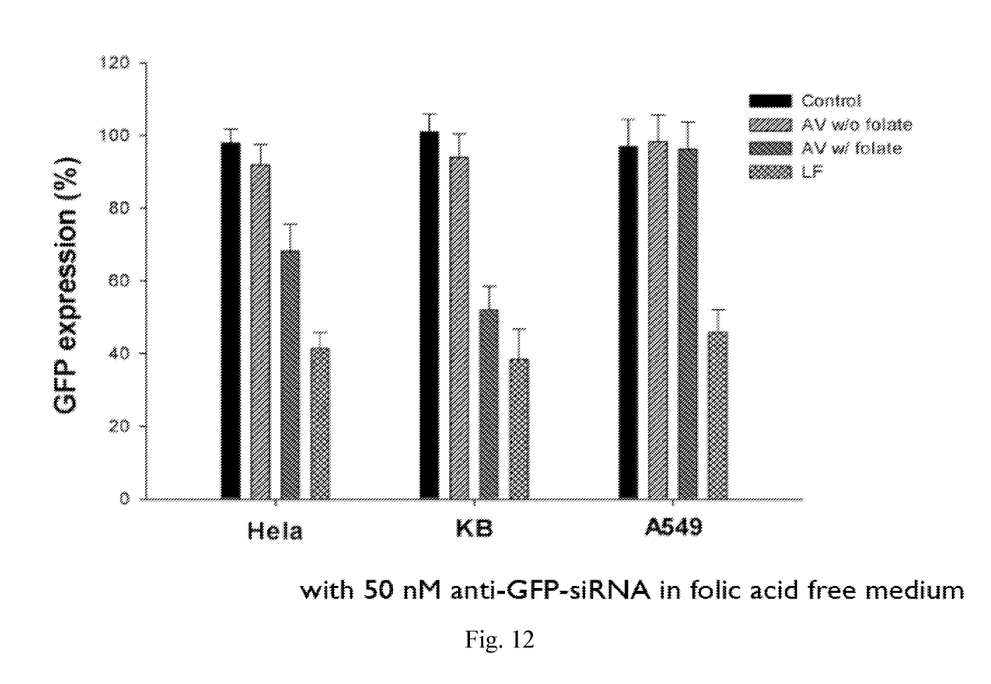

FIG. 13: Depicts GFP expression after delivery of anti-GFP siRNA in accordance with the present invention.

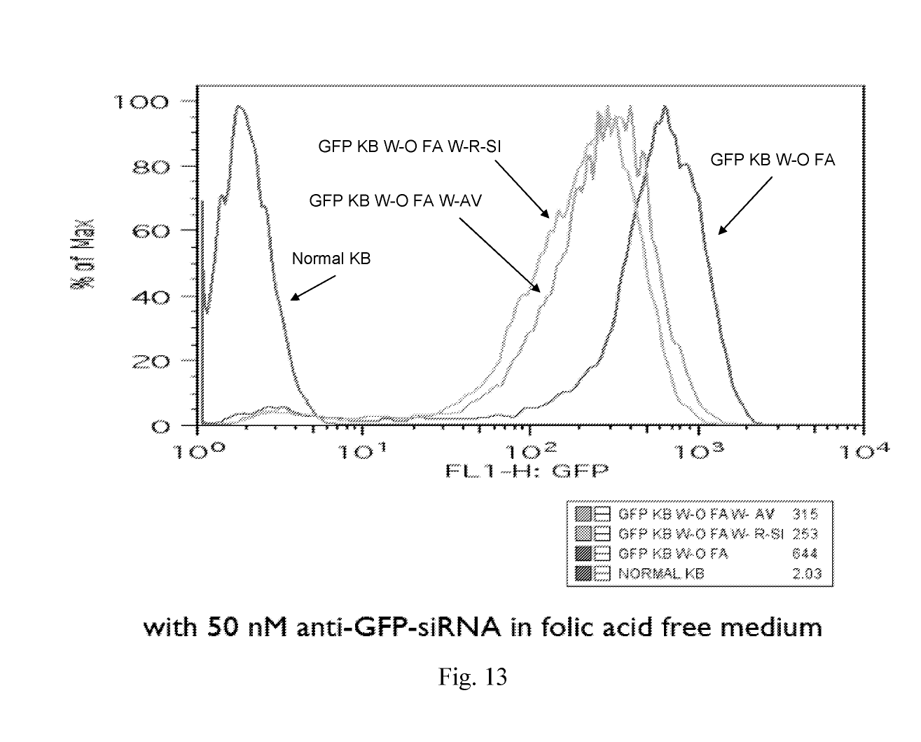

FIG. 14: Demonstrates assembly of Folate conjucted siRNA attached to a 6-nick tetrahedral delivery system as described herein.

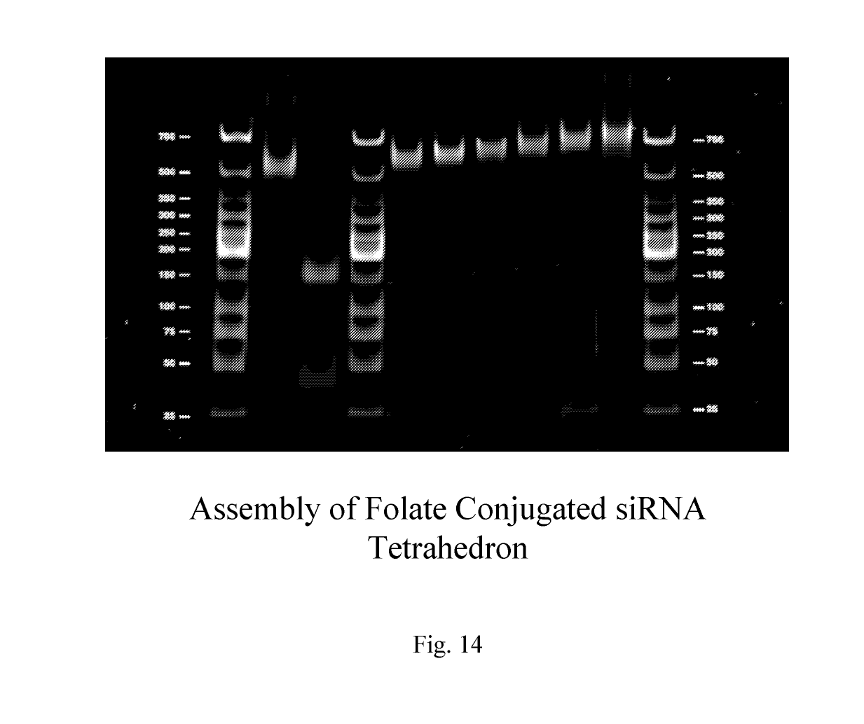

FIG. 15: Illustrates effect of the number of Folate conjugated siRNA hybridization on gene silencing

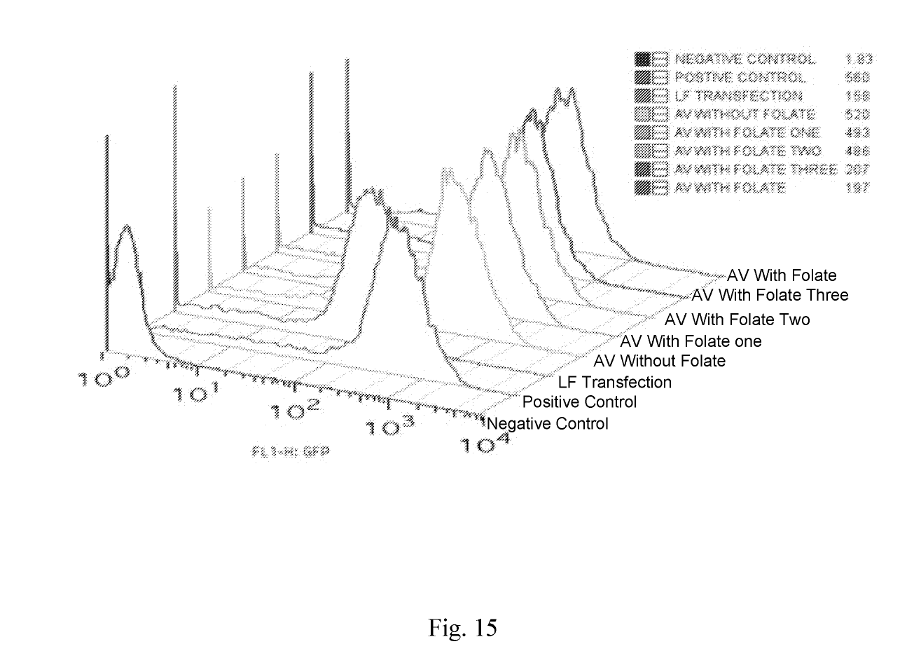

FIG. 16: Demonstrates the assembly of higher order tetrahedral structures as described herein.

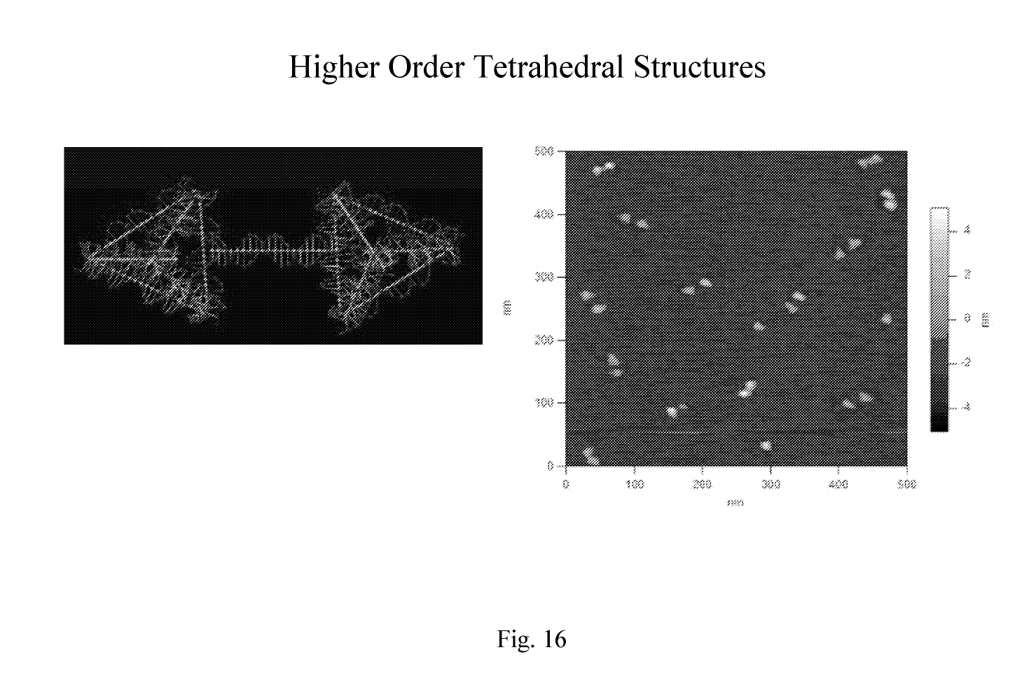

FIG. 17: Depicts an artificial virus comprised of 7 delivery systems as described herein and siRNA to be delivered; the outer layer can be associated with one or more targeting and/or cell penetrating domains.

FIG. 18: Illustrates the formation of an artificial virus comprised of 7 delivery systems as described herein and siRNA to be delivered

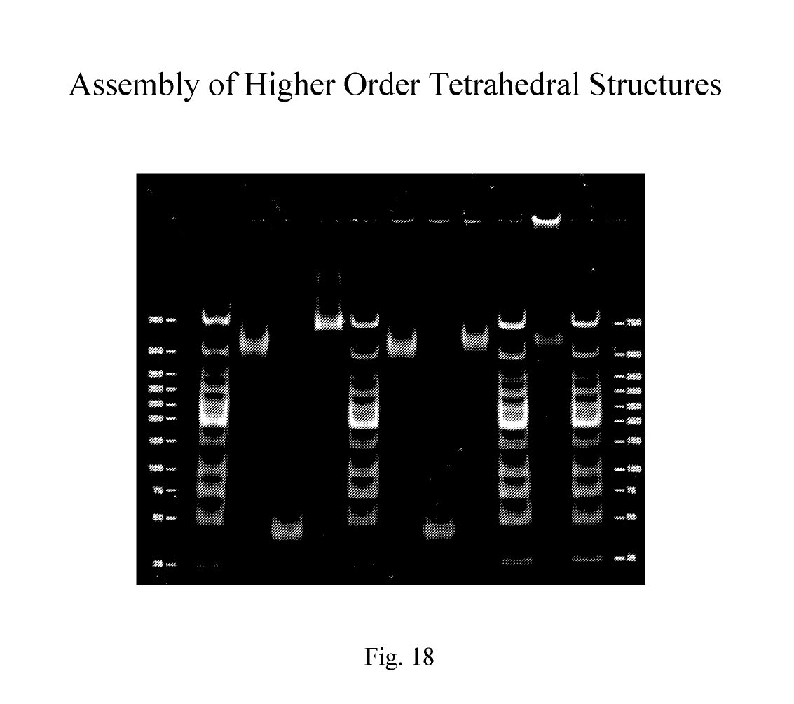

FIG. 19: Depicts design of DNA strands for tetrahedron formation (arrowed head stands for 5′ end of nucleic acid strand and each lower case letter correspond to the 6 edges of tetrahedron) and schematic representation showing site specific hybridization of siRNA to self-assembled nanoparticles; Panel B presents an AFM image showing mono-disperse tetrahedron DNA/siRNA nanoparticles

FIG. 20: Presents an AFM image showing mono-disperse tetrahedron DNA/siRNA nanoparticles

FIG. 21: Panel A depicts in vitro screening of tumor targeting ligands by Luc silencing in Hela cells; Panel B illustrates GFP gene silencing efficiency varies with FA density on ONPs; Panel C shows Structure/Function relation of ligand orientation and gene silencing efficiency of ONPs (set A: FA on 1, 2, and 3a; set B: FA on 1, 2, and 3b; set C: FA on 1, 2, and 3c); Panel D depicts results of automated confocal analysis of intracellular uptake of ONPs with different FA orientation.

FIG. 22: Panel A shows PK of ONPs in KB tumor-bearing mice and ex vivo fluorescence image of major organs and tumor at 12 h post-injection (T: Tumor, Lv: Liver, S: Spleen, K: Kidney, Lu: Lung, H: Heart); Panel B illustrates tumor specific accumulation of ONPs by FMT-CT (left: CT scan, right: 3D FMT-CT); Panel C depicts in vivo Luc silencing in KB tumor implants; Panel D shows quantitative analysis of Luc mRNA in KB tumors 2 days after ONP injection; Panel E presents in vivo live fluorescence images showing dose responsive accumulation of ONPs in KB tumors compared to naked folic acid conjugated siRNA (siRNA alone) through systemic injection.

FIG. 23: Panel A shows PAGE analysis of DNA tetrahedron formation and hybridization of siRNA to DNA cores; Panel B shows PAGE image confirming the full control of siRNA density on DNA tetrahedron.

FIG. 24: Depicts flow cytometry analysis of GFP gene silencing in GFP-KB cells by folic acid conjugated ONPs (35 nM).

FIG. 25: Panel A shows a time course siRNA concentration in blood after ONP injection (3 nmol of siRNA).

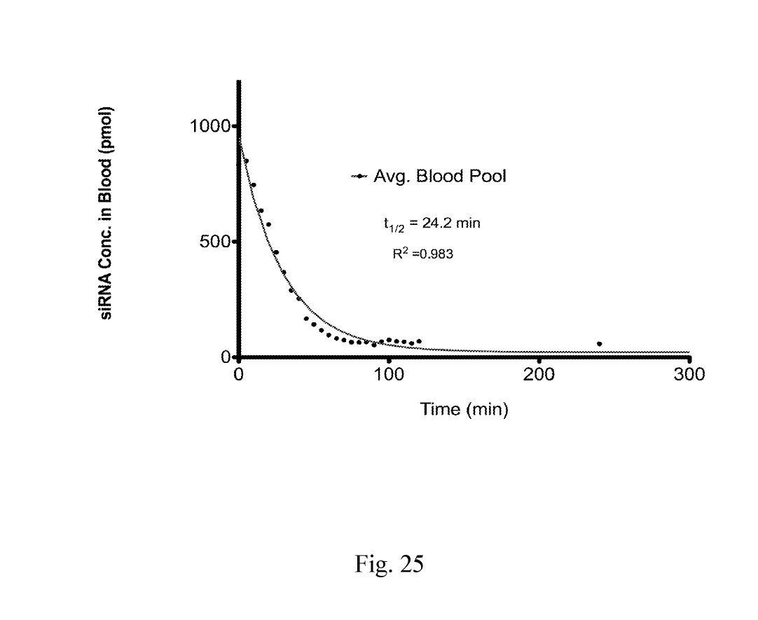

FIG. 26: Panel A shows dose responsive Luc silencing by systemic injection of ONPs and Panel B shows immune response data from in vivo study.

FIG. 27: Depicts alternative oligo-nucleic acid nanoparticle shapes that can be assembled using approaches as described herein.

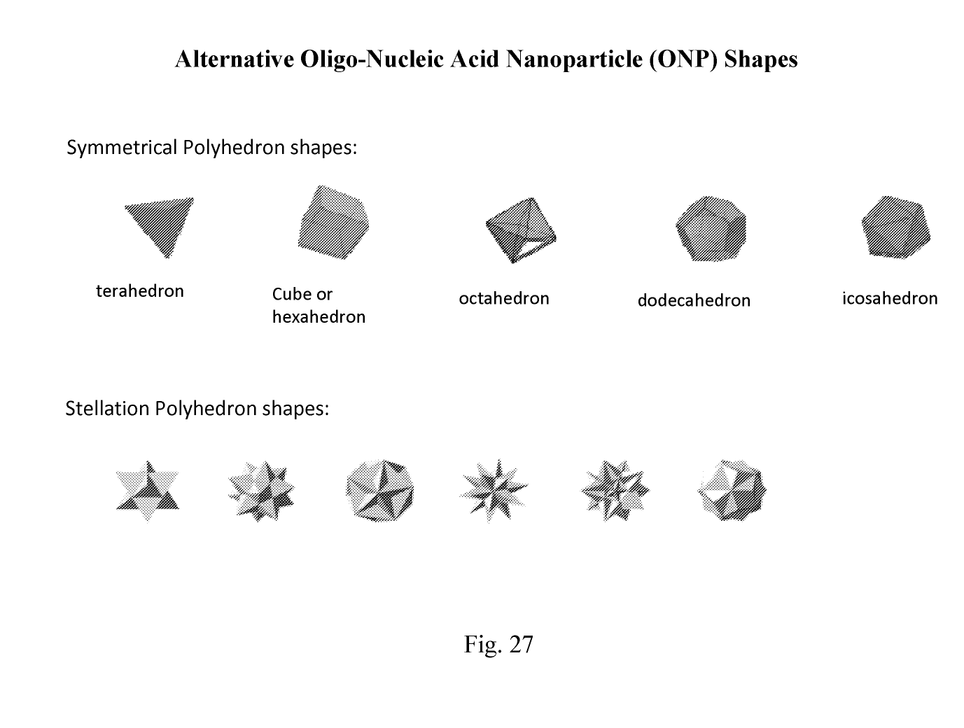

FIG. 28: Depicts alternative oligo-nucleic acid nanoparticle shapes that can be assembled using approaches as described herein.

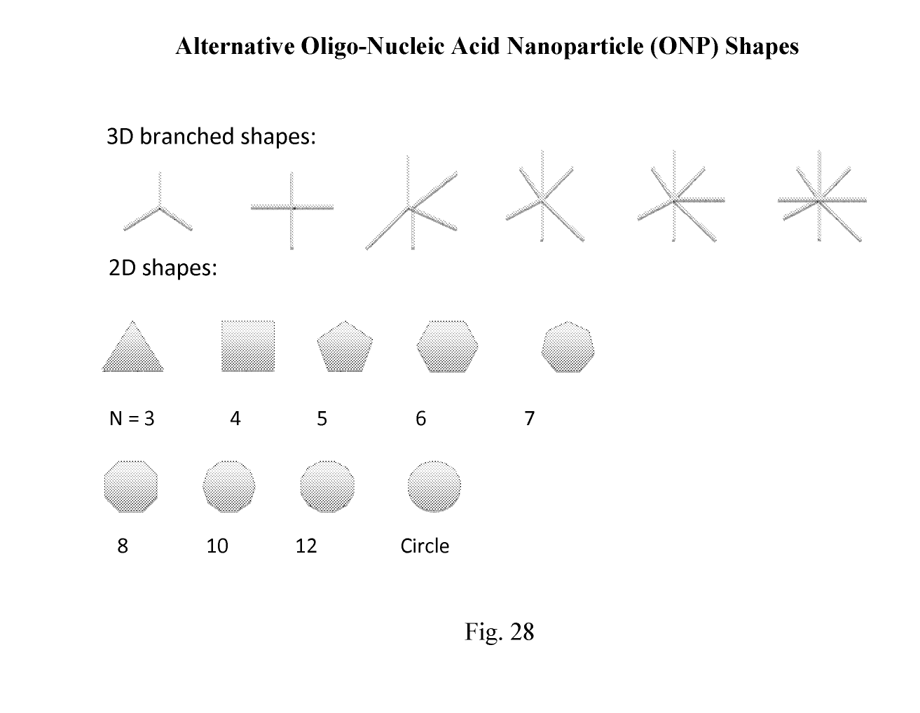

FIG. 29: Demonstrates different approaches for generating a double stranded siRNA molecule with an overhang, for attachment to the artificial virus.

FIG. 30: Illustrates GFP gene silencing efficiency using different approaches for attaching the siRNA to the ONP.

FIG. 31: Depicts preparation of a 6-nick tetrahedral delivery system according to the present invention from indicated oligonucleotides.

### DEFINITIONS

In order for the present invention to be more readily understood, certain terms are first defined. Additional definitions for the following terms and other terms are set forth throughout the specification.

Amplification: As used herein, the term “amplification” refers to any methods known in the art for copying a target nucleic acid, thereby increasing the number of copies of a selected nucleic acid sequence. Amplification may be exponential or linear. A target nucleic acid may be either DNA or RNA. Typically, the sequences amplified in this manner form an “amplicon.” Amplification may be accomplished with various methods including, but not limited to, the polymerase chain reaction (“PCR”), transcription-based amplification, isothermal amplification, rolling circle amplification, etc. Amplification may be performed with relatively similar amount of each primer of a primer pair to generate a double stranded amplicon. However, asymmetric PCR may be used to amplify predominantly or exclusively a single stranded product as is well known in the art (e.g., Poddar et al. Molec. And Cell. Probes 14:25-32 (2000)). This can be achieved using each pair of primers by reducing the concentration of one primer significantly relative to the other primer of the pair (e.g., 100 fold difference). Amplification by asymmetric PCR is generally linear. A skilled artisan will understand that different amplification methods may be used together.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Antibody: As used herein, the term “antibody” refers to a polypeptide consisting of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are typically classified as either kappa or lambda. Heavy chains are typically classified as gamma, mu, alpha, delta, or epsilon, which in turn define the immunoglobulin classes, IgG, IgM, IgA, IgD and IgE, respectively. A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms “variable light chain” (VL) and “variable heavy chain” (VH) refer to these light and heavy chains respectively. An antibody can be specific for a particular antigen. The antibody or its antigen can be either an analyte or a binding partner. Antibodies exist as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region thereby converting the (Fab′)2 dimer into an Fab′ monomer. The Fab′ monomer is essentially an Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of ordinary skill in the art will appreciate that such Fab′ fragments may be synthesized de novo either chemically or by utilizing recombinant DNA methodology. Thus, the term “antibody,” as used herein also includes antibody fragments either produced by the modification of whole antibodies or synthesized de novo using recombinant DNA methodologies. In some embodiments, antibodies are single chain antibodies, such as single chain Fv (scFv) antibodies in which a variable heavy and a variable light chain are joined together (directly or through a peptide linker) to form a continuous polypeptide. A single chain Fv (“scFv”) polypeptide is a covalently linked VH::VL heterodimer which may be expressed from a nucleic acid including VH- and VL-encoding sequences either joined directly or joined by a peptide-encoding linker. (See, e.g., Huston, et al. (1988) Proc. Nat. Acad. Sci. USA, 85:5879-5883, the entire contents of which are herein incorporated by reference.) A number of structures exist for converting the naturally aggregated, but chemically separated light and heavy polypeptide chains from an antibody V region into an scFv molecule which will fold into a three dimensional structure substantially similar to the structure of an antigen-binding site. See, e.g. U.S. Pat. Nos. 5,091,513 and 5,132,405 and 4,956,778.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: The term “associated with”, in its most general sense, refers to any direct or indirect attachment between two (or more) entities. In some embodiments, the entities are directly associated with one another in that there is no intervening entity (e.g., linker). In some embodiments, entities are considered to be directly associated with one another if they are covalently bound to one another. In some embodiments, an association is or comprises a covalent bond. In some embodiments, an association is or comprises one or more non-covalent interactions (e.g., involving one or more of hydrophobic forces, van der Waals forces, hydrogen bonds, magnetic interactions, etc). In some embodiments, associated entities are reversibly associated with one another in that the association can be disrupted under certain (typically predetermined) conditions. In some embodiments, the reversible associations are associations selected from the group consisting of electrostatic bonding, hydrogen bonding, van der Walls forces, ionic interaction, or donor/acceptor bonding. In some embodiments, the reversible association can include a combination of interactions, such as a combination of hydrogen bonding and ionic bonding, etc. In some embodiments, entities are irreversibly associated with one another. In some embodiments, an association involves specific binding.

Available for hybridization: The term “available for hybridization” is used herein to refer to nucleic acid sequence elements whose sequence is complementary to a ligand nucleic acid and that are capable of hybridization to such ligand nucleic acid in that it is in a state where such hybridization is not blocked. A nucleic acid sequence element may be considered “available for hybridization” with its cognate ligand nucleic acid when it is in fact so hybridized.

Coding sequence vs. non-coding sequence: As used herein, the term “coding sequence” refers to a sequence of a nucleic acid or its complement, or a part thereof, that can be transcribed and/or translated to produce the mRNA for and/or the polypeptide or a fragment thereof. Coding sequences include exons in a genomic DNA or immature primary RNA transcripts, which are joined together by the cell's biochemical machinery to provide a mature mRNA. The anti-sense strand is the complement of such a nucleic acid, and the encoding sequence can be deduced therefrom. As used herein, the term “non-coding sequence” refers to a sequence of a nucleic acid or its complement, or a part thereof, that is not transcribed into amino acid in vivo, or where tRNA does not interact to place or attempt to place an amino acid. Non-coding sequences include both intron sequences in genomic DNA or immature primary RNA transcripts, and gene-associated sequences such as promoters, enhancers, silencers, etc.

Complement: As used herein, the terms “complement,” “complementary” and “complementarity,” refer to the pairing of nucleotide sequences according to Watson/Crick pairing rules. For example, a sequence 5′-GCGGTCCCA-3′ has the complementary sequence of 5′-TGGGACCGC-3′. A complement sequence can also be a sequence of RNA complementary to the DNA sequence. Certain bases not commonly found in natural nucleic acids may be included in the complementary nucleic acids including, but not limited to, inosine, 7-deazaguanine, Locked Nucleic Acids (LNA), and Peptide Nucleic Acids (PNA). Complementary need not be perfect; stable duplexes may contain mismatched base pairs, degenerative, or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs.

Compound and Agent: The terms “compound” and “agent” are used herein interchangeably. They refer to any naturally occurring or non-naturally occurring (i.e., synthetic or recombinant) molecule, such as a biological macromolecule (e.g., nucleic acid, polypeptide or protein), organic or inorganic molecule, or an extract made from biological materials such as bacteria, plants, fungi, or animal (particularly mammalian, including human) cells or tissues. The compound may be a single molecule or a mixture or complex of at least two molecules.

Control: As used herein, the term “control” has its art-understood meaning of being a standard against which results are compared. Typically, controls are used to augment integrity in experiments by isolating variables in order to make a conclusion about such variables. In some embodiments, a control is a reaction or assay that is performed simultaneously with a test reaction or assay to provide a comparator. In one experiment, the “test” (i.e., the variable being tested) is applied. In the second experiment, the “control,” the variable being tested is not applied. In some embodiments, a control is a historical control (i.e., of a test or assay performed previously, or an amount or result that is previously known). In some embodiments, a control is or comprises a printed or otherwise saved record. A control may be a positive control or a negative control.

Deletion: As used herein, the term “deletion” encompasses a mutation that removes one or more nucleotides from a naturally-occurring nucleic acid.

Detect: As used herein, the term “detect” refers to a determination of whether a particular entity or moiety is present or absent, and or to a determination of a level at which the entity or moiety is present, and/or to a determination of a change in such level. Detection may involve a detectable or selectable marker, and/or an identifiable characteristic such as a fluorescent or radioactive moiety. Detection may involve analysis of a sample, for example using one or more techniques such as mass spectrometry or related methods, electrophoretic methods, nuclear magnetic resonance, chromatographic methods, FRET, and/or combinations thereof.

Epitope: As used herein, the term “epitope” refers to a fragment or portion of a molecule or a molecule compound (e.g., a polypeptide or a protein complex) that makes contact with a particular antibody or antibody like proteins.

Flanking: As used herein, the term “flanking” is meant that a primer hybridizes to a target nucleic acid adjoining a region of interest sought to be amplified on the target. The skilled artisan will understand that preferred primers are pairs of primers that hybridize 3′ from a region of interest, one on each strand of a target double stranded DNA molecule, such that nucleotides may be add to the 3′ end of the primer by a suitable DNA polymerase. For example, primers that flank mutant CTFR sequences do not actually anneal to the mutant sequence but rather anneal to sequence that adjoins the mutant sequence. In some cases, primers that flank a CFTR exon are generally designed not to anneal to the exon sequence but rather to anneal to sequence that adjoins the exon (e.g. intron sequence). However, in some cases, amplification primer may be designed to anneal to the exon sequence.

Hybridizing: The term “hybridizing” refers to the binding of two single stranded nucleic acids via complementary base pairing. The term “specific hybridization” refers to a process in which a nucleic acid molecule preferentially binds, duplexes, or hybridizes to a particular nucleic acid sequence under stringent conditions (e.g., in the presence of competitor nucleic acids with a lower degree of complementarity to the hybridizing strand). In certain embodiments of the present invention, these terms more specifically refer to a process in which a nucleic acid fragment (or segment) from a test sample preferentially binds to a particular probe and to a lesser extent or not at all, to other probes, for example, when these probes are immobilized on an array.

Isolated: As used herein, the term “isolated” refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from at least about 10%, about 20%, about 30%, about 40%, about 50%, about 60%, about 70%, about 80%, about 90%, about 95%, about 98%, about 99%, substantially 100%, or 100% of the other components with which they were initially associated. In some embodiments, isolated agents are more than about 80%, about 85%, about 90%, about 91%, about 92%, about 93%, about 94%, about 95%, about 96%, about 97%, about 98%, about 99%, substantially 100%, or 100% pure. As used herein, a substance is “pure” if it is substantially free of other components. As used herein, the term “isolated cell” refers to a cell not contained in a multi-cellular organism.

Kit: As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to a delivery systems comprising two or more separate containers that each contain a subportion of the total kit components. The containers may be delivered to the intended recipient together or separately. for example, a first container may contain an enzyme for use in an assay, while a second container contains oligonucleotides. The term “fragmented kit” is intended to encompass kits containing Analyte specific reagents (ASR's) regulated under section 520(e) of the Federal Food, Drug, and Cosmetic Act, but are not limited thereto. Indeed, any delivery system comprising two or more separate containers that each contain a subportion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits.

Label: In general, a “label” is any aspect or entity susceptible to being detected. To give but a few examples, a label may be or be or comprise a color, a tag, a fluorophore, a radioactive moiety, an epitope (e.g., recognized by an antibody), an oligonucleotide or other specific binding partner, a bar code, etc. An entity is considered to be “labeled” if it is associated with a label or with an agent that itself, or together with other agents, generates a label. In some embodiments, a label may be or comprise a dye or mixture of dyes. Dyes may be or comprise, for example, fluorescent dyes, chromophores or phosphors, among others. Dyes may be used individually and/or in mixtures. By varying the composition of the mixture (i.e. the ratio of one dye to another) and/or the concentration of a wide range of different possible labels can be constructed from a relatively small number of dyes. Suitable exemplary dyes for use in accordance with the present disclosure include, but are not limited to, fluorescent lanthanide complexes, including those of Europium and Terbium, fluorescein, rhodamine, tetramethylrhodamine, eosin, erythrosin, coumarin, methyl-coumarins, pyrene, Malacite green, stilbene, Lucifer Yellow, Cascade Blue™, Texas Red, and others (see, for example, the 1989-1991 Molecular Probes Handbook by Richard P. Haugland). In some embodiments, the term “primary label” is used to refer to entities (e.g., radioisotopes (e.g., ³²P, ³³P, ³⁵S, or ¹⁴C), mass-tags, fluorescent labels, etc) that signal generating reporter groups which can be detected without further modifications. In some embodiments, the term “secondary label” is used to refer to entities, such as biotin and various protein antigens, that require the presence of a second intermediate for production of a detectable signal. For biotin, for example, the secondary intermediate may include streptavidin-enzyme conjugates. For antigen labels, secondary intermediates may include, for example, antibody-enzyme conjugates. Some fluorescent groups act as secondary labels because they transfer energy to another group in the process of nonradiative fluorescent resonance energy transfer (FRET), and the second group produces the detected signal. Those of ordinary skill in the art will appreciate that a “fluorescent label”, as used herein, is one that absorbs light energy at a defined excitation wavelength and emit light energy at a different wavelength. Examples of fluorescent labels include, but are not limited to: Alexa Fluor dyes (Alexa Fluor 350, Alexa Fluor 488, Alexa Fluor 532, Alexa Fluor 546, Alexa Fluor 568, Alexa Fluor 594, Alexa Fluor 633, Alexa Fluor 660, and Alexa Fluor 680), AMCA, AMCA-S, BODIPY dyes (BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, and BODIPY 650/665), Carboxyrhodamine 6G, carboxy-X-rhodamine (ROX), Cascade Blue, Cascade Yellow, Coumarin 343, Cyanine dyes (Cy3, Cy5, Cy3.5, and Cy5.5), Dansyl, Dapoxyl, Dialkylaminocoumarin, 4′,5′-Dichloro-2′,7′-dimethoxy-fluorescein, DM-NERF, Eosin, Erythrosin, Fluorescein, FAM, Hydroxycoumarin, IRDyes (IRD40, IRD 700, and IRD 800), JOE, Lissamine rhodamine B, Marina Blue, Methoxycoumarin, Naphthofluorescein, Oregon Green 488, Oregon Green 500, Oregon Green 514, Pacific Blue, PyMPO, Pyrene, Rhodamine B, Rhodamine 6G, Rhodamine Green, Rhodamine Red, Rhodol Green, 2′,4′,5′,7′-Tetra-bromosulfone-fluorescein, Tetramethyl-rhodamine (TMR), Carboxytetramethylrhodamine (TAMRA), Texas Red, and Texas Red-X.

Mass tag: The term “mass-tag” as used herein refers to any moiety that is capable of being uniquely detected by virtue of its mass using mass spectrometry (MS) detection techniques. Examples of mass-tags include electrophore release tags such as N-[3-[4′-[(p-methoxytetrafluorobenzyl)oxy]phenyl]-3-methylglyceronyl]isonipecotic acid, 4′-[2,3,5,6-tetrafluoro-4-(pentafluorophenoxyl)]methyl acetophenone, and their derivatives. The synthesis and utility of these mass-tags is described in U.S. Pat. Nos. 4,650,750, 4,709,016, 5,360,8191, 5,516,931, 5,602,273, 5,604,104, 5,610,020, and 5,650,270. Other examples of mass-tags include, but are not limited to, nucleotides, dideoxynucleotides, oligonucleotides of varying length and base composition, oligopeptides, oligosaccharides, and other synthetic polymers of varying length and monomer composition. A large variety of organic molecules, both neutral and charged (biomolecules or synthetic compounds) of an appropriate mass range (100-2000 Daltons) may also be used as mass-tags.

Nucleic Acid: As used herein refers to an oligonucleotide, nucleotide or polynucleotide, and fragments or portions thereof, and to DNA or RNA of genomic or synthetic origin that may be single or double stranded, and represent the sense or antisense strand.

Nucleic Acid Molecule: The terms “nucleic acid molecule” and “polynucleotide” are used herein interchangeably. They refer to a deoxyribonucleotide or ribonucleotide polymer in either single- or double-stranded form, and unless otherwise stated, encompass known analogs of natural nucleotides that function in a similar manner as naturally occurring nucleotides, for example sharing an ability to be incorporated into a polynucleotide chain and/or to hybridize with bases in a polynucleotide. In some embodiments, the terms refer to nucleic acid-like structures with synthetic backbones. In some embodiments, the terms refer to amplification products. Typically, a nucleic acid molecule has a 5′ end and a 3′ end. A first region along a nucleic acid strand is said to be upstream of another region if the 3′ end of the first region is before the 5′ end of the second region when moving along a strand of nucleic acid in a 5′ to 3′ direction. For example, when two different, non-overlapping oligonucleotides anneal to different regions of the same linear complementary nucleic acid sequence, and the 3′ end of one oligonucleotide points towards the 5′ end of the other, the former may be called the “upstream” oligonucleotide and the latter the “downstream” oligonucleotide. Similarly, when two overlapping oligonucleotides are hybridized to the same linear complementary nucleic acid sequence, with the first oligonucleotide positioned such that its 5′ end is upstream of the 5′ end of the second oligonucleotide, and the 3′ end of the first oligonucleotide is upstream of the 3′ end of the second oligonucleotide, the first oligonucleotide may be called the “upstream” oligonucleotide and the second oligonucleotide may be called the “downstream” oligonucleotide.

Nucleotide Analog: The term “nucleotide analog” as used herein refers to modified or non-naturally occurring nucleotides including but not limited to analogs that have altered stacking interactions such as 7-deaza purines (i.e., 7-deaza-dATP and 7-deaza-dGTP); base analogs with alternative hydrogen bonding configurations (e.g., such as Iso-C and Iso-G and other non-standard base pairs described in U.S. Pat. No. 6,001,983 to S. Benner and herein incorporated by reference); non-hydrogen bonding analogs (e.g., non-polar, aromatic nucleoside analogs such as 2,4-difluorotoluene, described by B. A. Schweitzer and E. T. Kool, J. Org. Chem., 1994, 59, 7238-7242, B. A. Schweitzer and E. T. Kool, J. Am. Chem. Soc., 1995, 117, 1863-1872; each of which is herein incorporated by reference); “universal” bases such as 5-nitroindole and 3-nitropyrrole; and universal purines and pyrimidines (such as “K” and “P” nucleotides, respectively; P. Kong, et al., Nucleic Acids Res., 1989, 17, 10373-10383, P. Kong et al., Nucleic Acids Res., 1992, 20, 5149-5152). Nucleotide analogs include nucleotides having modification on the sugar moiety, such as dideoxy nucleotides and 2′-O-methyl nucleotides. Nucleotide analogs include modified forms of deoxyribonucleotides as well as ribonucleotides.

Protein: In general, a “protein” is a polypeptide (i.e., a string of at least two amino acids linked to one another by peptide bonds). Proteins may include moieties other than amino acids (e.g., may be glycoproteins) and/or may be otherwise processed or modified. Those of ordinary skill in the art will appreciate that a “protein” can be a complete polypeptide chain as produced by a cell (with or without a signal sequence), or can be a functional portion thereof. Those of ordinary skill will further appreciate that a protein can sometimes include more than one polypeptide chain, for example linked by one or more disulfide bonds or associated by other means.

siRNA: The term “siRNAs” refers to short interfering RNAs. In some embodiments, siRNAs comprise a duplex, or double-stranded region, where each strand of the double-stranded region is about 18 to 25 nucleotides long; the double-stranded region can be as short as 16, and as long as 29, base pairs long, where the length is determined by the antisense strand. Often siRNAs contain from about two to four unpaired nucleotides at the 3′ end of each strand. SiRNAs appear to function as key intermediates in triggering RNA interference in invertebrates and in vertebrates, and in triggering sequence-specific RNA degradation during posttranscriptional gene silencing in plants. At least one strand of the duplex or double-stranded region of a siRNA is substantially homologous to or substantially complementary to a target RNA molecule. The strand complementary to a target RNA molecule is the “antisense” strand; the strand homologous to the target RNA molecule is the “sense” strand and is also complementary to the siRNA antisense strand. One strand of the double stranded region need not be the exact length of the opposite strand′ thus, one strand may have at least one fewer nucleotides than the opposite complementary strand, resulting in a “bubble” or at least one unmatched base in the opposite strand. One strand of the double-stranded region need not be exactly complementary to the opposite strand; thus, the strand, preferably the sense strand, may have at least one mismatched base pair. In some embodiments, an siRNA for use in accordance with the present invention contains one or more additional sequences; non-limiting examples of such sequences include linking sequences, or loops, which connect the two strands of the duplex region. This form of siRNAs may be referred to “si-like RNA”, “short hairpin siRNA” where the short refers to the duplex region of the siRNA, or “hairpin siRNA”. Additional non-limiting examples of additional sequences present in siRNAs include stem and other folded structures. Such additional sequences may or may not have known functions; non-limiting examples of such functions include increasing stability of an siRNA molecule, or providing a cellular destination signal.

Substantially complementary: As used herein, the term “substantially complementary” refers to two sequences that can hybridize under stringent hybridization conditions. The skilled artisan will understand that substantially complementary sequences need not hybridize along their entire length. In some embodiments, “stringent hybridization conditions” refer to hybridization conditions at least as stringent as the following: hybridization in 50% formamide, 5×SSC, 50 mM NaH2PO4, pH 6.8, 0.5% SDS, 0.1 mg/mL sonicated salmon sperm DNA, and 5×Denhart's solution at 42° C. overnight; washing with 2×SSC, 0.1% SDS at 45° C.; and washing with 0.2×SSC, 0.1% SDS at 45° C. In some embodiments, stringent hybridization conditions should not allow for hybridization of two nucleic acids which differ over a stretch of 20 contiguous nucleotides by more than two bases.

Sample: As used herein, the term “Sample” encompasses any sample obtained from a biological source. The terms “biological sample” and “sample” are used interchangeably. A biological sample can, by way of non-limiting example, include cerebrospinal fluid (CSF), blood, amniotic fluid, sera, urine, feces, epidermal sample, skin sample, cheek swab, sperm, amniotic fluid, cultured cells, bone marrow sample and/or chorionic villi. Convenient biological samples may be obtained by, for example, scraping cells from the surface of the buccal cavity. Cell cultures of any biological samples can also be used as biological samples, e.g., cultures of chorionic villus samples and/or amniotic fluid cultures such as amniocyte cultures. A biological sample can also be, e.g., a sample obtained from any organ or tissue (including a biopsy or autopsy specimen), can comprise cells (whether primary cells or cultured cells), medium conditioned by any cell, tissue or organ, tissue culture. In some embodiments, biological samples suitable for the invention are samples which have been processed to release or otherwise make available a nucleic acid for detection as described herein. Suitable biological samples may be obtained from a stage of life such as a fetus, young adult, adult (e.g., pregnant women), and the like. Fixed or frozen tissues also may be used.

System: The term “system” and “biological system” are used herein interchangeably. A system may be any biological entity that can express or comprise at least one inventive biomarker. In the context of the present invention, in vitro, in vivo, and ex vivo systems are considered; and the system may be a cell, a biological fluid, a biological tissue, or an animal. For example, a system may originate from a living subject (e.g., it may be obtained by drawing blood, or by performing needle biopsy), or from a deceased subject (e.g., it may be obtained at autopsy).

Therapeutic agent: As used herein, the phrase “therapeutic agent” refers to any agent that elicits a desired pharmacological effect when administered to an organism. In some embodiments, an agent is considered to be a therapeutic agent if it demonstrates a statistically significant effect across an appropriate population. In some embodiments, the appropriate population may be a population of model organisms. In some embodiments, an appropriate population may be defined by various criteria, such as a certain age group, gender, genetic background, preexisting clinical conditions, etc. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition.

Treatment: As used herein, the term “treatment” refers to a therapeutic protocol that alleviates, delays onset of, reduces severity or incidence of, and/or yield prophylaxis of one or more symptoms or aspects of a disease, disorder, or condition. In some embodiments, treatment is administered before, during, and/or after the onset of symptoms. In some embodiments, treatment may be administered to a subject who does not exhibit signs of a disease, disorder, and/or condition. In some embodiments, treatment may be administered to a subject who exhibits only early signs of the disease, disorder, and/or condition, for example for the purpose of decreasing risk of developing pathology associated with the disease, disorder, and/or condition.

### DETAILED DESCRIPTION OF CERTAIN EMBODIMENTS

Oligonucleotide Delivery Systems

As described herein, the present invention provides nanoparticle delivery systems comprised of oligonucleotides. In some embodiments, provided oligonucleotides self-assemble into first order three-dimensional structures with nanoscale dimensions (i.e., nanoparticles). In some embodiments, such first order three-dimensional nanoparticle structures assemble into higher-order structures. In some embodiments, even the higher-order structures have nanoscale dimensions. For example, in some embodiments, provided oligonucleotides assemble (e.g., self-assemble) into first order and/or higher order structures that are under 500 nm, 400 nm, 300 nm, 200 nm, 100 nm, 90 nm, 80 nm, 70 nm, 60 nm, 50 nm, 40 nm, 30 nm, 20 nm, 10 nm, or even 5 nm in size. In some embodiments, provided oligonucleotides assemble into first order and/or higher order structures that are within the range of 6 nm-100 nm in size. In some embodiments, provided oligonucleotides assemble into structures whose size is within a range having a lower limit of 5, 6, 7, 8, 9, 10, 11, 12, 13, 14, 15, 16, 17, 18, 19, 20, 25, 30, 25, 40, 45, 50, 55, 60, 65, 70, 75, 80, 85, 90, 95, 96, 97, 98, 99, 100 nm or more, and an upper limit of 500, 450, 400, 350, 300, 250, 200, 150, 100, 95, 94, 93, 92, 91, 90 nm or less.

DNA is known to be able to assemble into a wide range of nanostructures (see, for example, Seeman, N. C. DNA in a material world. Nature 421, 427-431 (2003), Seeman, N. C. DNA enables nanoscale control of the structure of matter. Q. Rev., Biophys. 38, 363-371 (2005), Feldkamp, U. & Niemeyer, C. M. Rational design of DNA nanoarchitecture, Angew. Chem. Int. Edn Engl. 45, 1856-1876 (2006), Adleman, L. M. Molecular computation of solutions to combinatorial problems, Science 266, 1021-1024 (1994), Winfree, E., Liu, F. R., Wenzler, L. A. & Seeman, N. C. Design and self-assembly of two-dimensional DNA crystals. Nature 394, 539-544 (1998)., Rothemund, P. W. K., Papadakis, N. & Winfree, E. Algorithmic self-assembly of DNA Sierpinski triangles. PLoS Biol. 2, 2041-2053 (2004), Yan, H., Park, S. H., Finkelstein, G., Reif, J. H. & LaBean, T. H. DNA-templated selfassembly of protein arrays and highly conductive nanowires. Science 301, 1882-1884 (2003), Scheffler, M., Dorenbeck, A., Jordan, S., Wustefeld, M. & von Kiedrowski, G. Selfassembly of trisoligo-nucleotidyls: the case for nano-acetylene and nanocyclobutadiene. Angew. Chem. Int. Edn Engl. 38, 3312-3315 (1999), Rothemund, P. W. K. Folding DNA to create nanoscale shapes and patterns. Nature 440, 297-302 (2006), Chen, J. H. & Seeman, N. C. Synthesis from DNA of a molecule with the connectivity of a cube. Nature 350, 631-633 (1991), Zhang, Y. W. & Seeman, N. C. Construction of a DNA-truncated octahedron. J. Am. Chem. Soc. 116, 1661-1669 (1994), Shih, W. M., Quispe, J. D. & Joyce, G. F. A 1.7-kilobase single-stranded DNA that folds into a nanoscale octahedron. Nature 427, 618-621 (2004), Goodman, R. P. et al. Rapid chiral assembly of rigid DNA building blocks for molecular nanofabrication. Science 310, 1661-1665 (2005), and Goodman, R. P., Berry, R. M. & Turberfield, A. J. The single-step synthesis of a DNA tetrahedron. Chem. Commun 1372-1373 (2004); including cubes (Chen, J. H. & Seeman, N. C. Synthesis from DNA of a molecule with the connectivity of a cube. Nature 350, 631-633 (1991), truncated octahedral (Zhang, Y. W. & Seeman, N. C. Construction of a DNA-truncated octahedron. J. Am. Chem. Soc. 116, 1661-1669 (1994), ocrohedra (Shih, W. M., Quispe, J. D. & Joyce, G. F. A 1.7-kilobase single-stranded DNA that folds into a nanoscale octahedron. Nature 427, 618-621 (2004), and tetrahedra (Goodman, R. P. et al. Rapid chiral assembly of rigid DNA building blocks for molecular nanofabrication. Science 310, 1661-1665 (2005), Goodman, R. P., Berry, R. M. & Turberfield, A. J. The single-step synthesis of a DNA tetrahedron. Chem. Commun 1372-1373 (2004)). Many of these structures have been exemplified in the literature through assembly of a plurality of oligonucleotides having unique sequences. Work has also been done to establish that sets of oligonucleotides of identical sequence can also self-assemble into three-dimensional structures such as, for example, tetrahedral, dodecahedra, and buckyballs (see, for example, Goodman et al., Science 310:1661, Dec. 9, 2005; He et al., Nature 452:198, Mar. 13, 2008, each of which is incorporated herein by reference in its entirety). In many such embodiments, the three-dimensional structures are tens of nanometers in size

RNA is also known to be able to assemble into various nanostructures. For example, packaging RNA (pRNA) of bacteriophage phi29 assembles into a unique 11 nm particle (see, for example, Guo et al., Mol Cell 2:149, 1998; Hoeprich et al., J Biol Chem 277:20749, 2002; Gue et al., Nat Nanotechnology 5: 1964, 2005; Guo et al., Adv Drug Deliv 62:650, 2010; Abdelmawla et al., Mol Therap doi:10.1038/mt.2011.35, Apr. 5, 2011, each of which is incorporated herein by reference in its entirety). pRNA is a component of the bacteriophage phi29 DNA-packaging motor. pRNA can form dimers, trimers, and hexamers. Each pRNA contains two functional domains. The central domain of pRNA contains two interlocking left hand and right hand loops that can be engineered to form stable intermolecular loop-loop interactions. The DNA-packaging domain is located at the 5′/3′ paired ends. The two domains fold separately, and replacement of the packaging domain with siRNA does not affect pRNA structure, folding, or intermolecular interactions. The resultant pRNA/siRNA chimera is useful for gene therapy.

Inventors notes that literature describing nucleic acid nanostructures, and particularly that describing higher-order nanostructures, emphasize desirability of and/or need for covalent linkages to establish higher-order nanostructures. Surprisingly, the present disclosure demonstrates that certain nanoparticles described herein show beneficial stability (as manifested, for example, through an ability to achieve successful delivery of particular payloads) without covalent association to generate higher-order structures. This feature of certain provided nanoparticles may provide additional advantages including, for example, ensuring feasibility of renal clearance (e.g., by permitting disassembly to sufficiently small structures even if untoward aggregation may have occurred). Moreover, provided nanoparticles can achieve higher-order assembly through self-assembly mechanisms, which are typically not available if covalent linkage is required.

Still further, the inventors note that literature describing higher-order assembly of nanoparticle nucleic acid structures often describes a requirement for face-to-face association in order to achieve such higher-order assembly. In some embodiments, provided nanoparticles achieve higher-order assembly at least in part through face-to-face association. In some embodiments, however, face-to-face association is not required and/or is not involved in higher-order assembly according to the present invention. For example, particularly in embodiments in which provided delivery systems comprise nanoparticles and payload moieties (e.g., targeting payload moieties) associated with one or more particular nanoparticle faces, face-to-face association may not be required for or involved in higher-order assembly. Indeed, in some embodiments, presence of one or more exterior payload moieties may reduce or abolish face-to-face association.

The present invention provides three dimensional nanoparticles comprised of a plurality of oligonucleotides whose sequences are selected so that they self-assemble into three-dimensional structures by hybridization of complementary subsequences. In some embodiments, each oligonucleotide in the plurality has a sequence including a portion that is complementary to a portion of at least one other oligonucleotide in the plurality. In some embodiments, the oligonucleotides in the plurality have sequences selected so that, when the oligonucleotides are hybridized to one another to form the three-dimensional structure, sides of the structure are substantially double stranded; in some embodiments one or more nicks may be present.

In some embodiments, at least some of the oligonucleotides in the plurality have a nucleotide sequence that includes a portion that is a ligand hybridization element in that it remains single stranded in the three-dimensional structure, so that it is available for hybridization with a complementary ligand sequence. In some embodiments, at least three oligonucleotides in the plurality have such a ligand hybridization element. In some embodiments, all oligonucleotides in the plurality have such a ligand hybridization element. In some embodiments, different oligonucleotides in the plurality have different such ligand hybridization elements, such that they hybridize with different complementary ligand sequences.

In some embodiments, oligonucleotides in the plurality have nucleotide sequences selected so that, when the oligonucleotides hybridize with one another and form the three-dimensional nanoparticle, the nanoparticle has a plurality of ligand hybridization elements. In some embodiments, oligonucleotides in the plurality have nucleotide sequences selected so that, when the oligonucleotides hybridize with one another and form the three-dimensional nanoparticle, the nanoparticle has a plurality of ligand hybridization elements arranged relative to one another so that, when ligands associated with payload moieties hybridize to the ligand hybridization elements, at least 2 such payload moieties are displayed on a single face of the nanoparticle.

In some embodiments, oligonucleotides in the plurality have nucleotide sequences selected so that, when the oligonucleotides hybridize with one another and form the three-dimensional nanoparticle, a plurality of ligand hybridization elements is located arms defining the same face of the three-dimensional structure. In some such embodiments, at least three ligand hybridization elements are located on arms defining the same face of the three-dimensional structure.

In some embodiments, oligonucleotides in the plurality have nucleotide sequences selected so that, when the oligonucleotides hybridize with one another and form the three-dimensional nanoparticle, the nanoparticle comprises a plurality of different ligand hybridization elements (i.e., ligand hybridization elements that differ from one another in length and/or sequence composition).

In some embodiments, oligonucleotides in the plurality have sequences selected so that ligand hybridization elements comprise one or more natural nucleotides. In some embodiments, oligonucleotides in the plurality have sequences selected so that ligand hybridization elements comprise one or more non-natural nucleotides. In some embodiments, oligonucleotides in the plurality have sequences selected so that ligand hybridization elements comprise poly-purine. In some embodiments, oligonucleotides in the plurality have sequences selected so that ligand hybridization elements comprise polyT or polyA.

In some embodiments, ligand hybridization elements are at least 10 nucleotide bases long. In some embodiments, ligand hybridization elements have a length within the range of 10 to 40 nucleotide bases long. In some embodiments, length of ligand hybridization elements is selected with consideration of ligand hybridization sequence, in order to achieve appropriate stability for in vivo delivery (see, for example, Example 3).

In some embodiments, one or more ligand hybridization elements is present as a single stranded nick or gap, e.g., in a nanoparticle arm. In some embodiments, one or more ligand hybridization elements is present as a single stranded portion (e.g., “overhang”), for example extending from an arm or point on a nanoparticle.

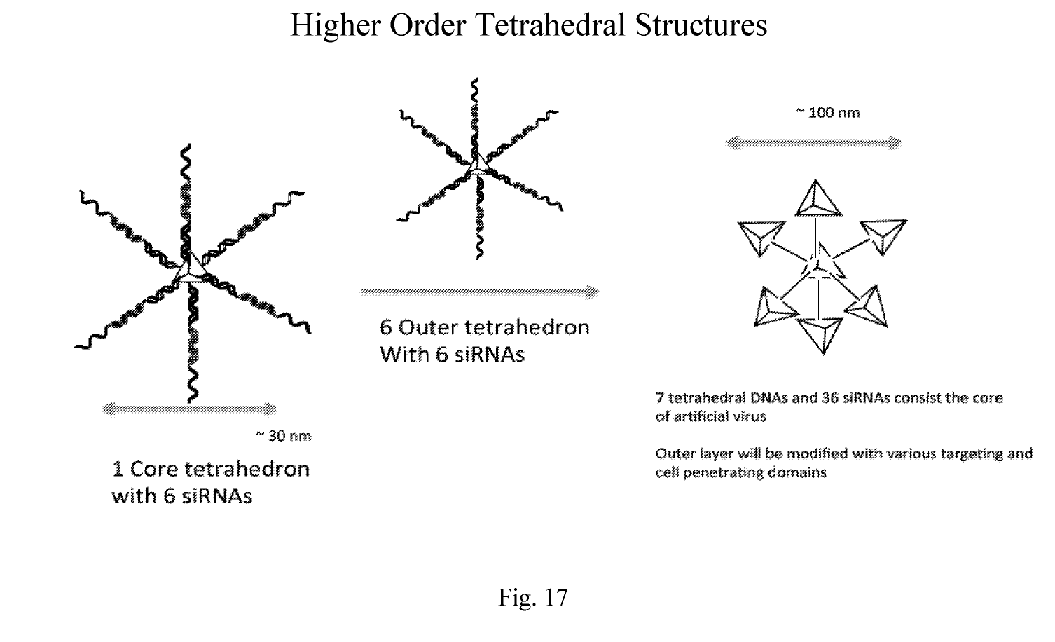

In some embodiments, ligand hybridization elements on different nanoparticles within a composition comprising a plurality of nanoparticles have complementary sequences such that nanoparticles in the plurality self-assemble into higher-order structures through hybridization of ligand hybridization elements with one another. In some embodiments, ligand hybridization elements on different nanoparticles within a composition comprising a plurality of nanoparticles have sequences selected to be complementary to a common “bridge” oligonucleotide, such that addition of the bridge oligonucleotide to the composition permits higher-order association of nanoparticles through common association of two or more nanoparticles in the composition with the same bridge oligonucleotide. In some embodiments, such a bridge oligonucleotide is or comprises a ligand oligonucleotide and/or a payload moiety (e.g., an siRNA, such as is illustrated, for example, in FIG. 17).

In some embodiments, oligonucleotides that assemble into delivery structures as provided herein have a length that is shorter than about 500 nt. In some embodiments, oligonucleotides that assemble into delivery structures as provided herein have a length that is shorter than 500, 450, 400, 350, 300, 250, 200, 150, 100, 95, 90, 85, 80, 75, 70, 65, 60, 55, 50, 45, 40, 35, 30, or fewer nucleotides. In some embodiments, oligonucleotides that assemble into delivery structures as provided herein have a length that is longer than about 5, 6, 7, 8, 9, 10, 11, 12, 13, 14, 15, 16, 17, 18, 19, 20, 21, 22, 23, 24, 25, 26, 27, 28, 29, 30 or more nucleotides. In some embodiments, oligonucleotides that assemble into delivery structures as provided herein have a length that is within the range of 5-100, 10-90, 10-80, 10-70, 10-60, 15-50, or 20-40 nucleotides.

In some embodiments, oligonucleotides as provided herein assemble into three-dimensional nanoparticles having at least two faces defined by substantially double-stranded arms. In some embodiments, a nanoparticle as described herein has at least 3, 4, 5, 6, 7, 8, 9, 10, 11, 12, 13, 14, 15, 16, 17, 18, 19, 20, 21, 22, 23, 24, 25 faces or more. In some embodiments, oligonucleotides as provided herein assemble into three-dimensional structures that are polyhedral structures, having 3, 4, 5, 6, 7, 8, 9, 10, 11, 12 or more arms.

In some embodiments, oligonucleotides as provided herein assemble into three-dimensional nanoparticles that define an interior and an exterior. In some such embodiments, oligonucleotides as provided herein assemble into three-dimensional nanoparticle comprising ligand hybridization elements arranged relative to one another such that, when ligand sequences associated with payload moieties hybridize to the ligand hybridization elements, at least one payload moiety is positioned in the nanoparticle interior. In some embodiments, oligonucleotides as provided herein assemble into three-dimensional nanoparticles that define an interior and an exterior. In some embodiments, oligonucleotides as provided herein assemble into three-dimensional nanoparticle comprising ligand hybridization elements arranged relative to one another such that, when ligand sequences associated with payload moieties hybridize to the ligand hybridization elements, at least a plurality payload moieties is positioned in the nanoparticle interior.

In some such embodiments, oligonucleotides as provided herein assemble into three-dimensional nanoparticle comprising ligand hybridization elements arranged relative to one another such that, when ligand sequences associated with payload moieties hybridize to the ligand hybridization elements, at least one payload moiety is positioned on the nanoparticle exterior. In some such embodiments, at least a plurality payload moieties is positioned on the nanoparticle exterior. In some such embodiments, at least three payload moieties are positioned in the nanoparticle exterior.

In some such embodiments, oligonucleotides as provided herein assemble into three-dimensional nanoparticle comprising ligand hybridization elements arranged relative to one another such that, when ligand sequences associated with payload moieties hybridize to the ligand hybridization elements, at least one payload moiety is positioned in the nanoparticle interior and at least one payload moiety is positioned on the nanoparticle exterior. In some such embodiments, at least a plurality (e.g., at least 3) of payload moieties is positioned on the nanoparticle exterior; alternatively or additionally, at least a plurality of payload moieties is positioned in the nanoparticle interior.

The present inventors note that certain literature in the field of delivery systems emphasize desirability of including delivered ligands only interior to delivery systems. The present invention, by contrast, specifically provides and encompasses delivery systems comprising payloads (including delivered payload moieties and/or payload moieties [e.g., certain targeting moieties] that may remain associated with the nanoparticle after delivery, etc) exterior to the nanoparticle, including, as described herein, delivery systems comprising both interior and exterior payload moieties.

Additional Components

In some embodiments, provided three-dimensional oligonucleotide structures are assembled together with one or more additional components comprising an oligonucleotide with a ligand sequence. In some such embodiments, the ligand sequence oligonucleotide is associated with at least one payload moiety. In some embodiments, the association is or comprises a covalent bond. In some embodiments, the association is or comprises one or more non-covalent interactions. In some embodiments, the association is direct. In some embodiments the association is indirect in that the oligonucleotide and the payload moiety are associated with one another by means of a biological or chemical linker. A wide variety of linkers is known in the art. In some such embodiments, the linker is cleavable. In some embodiments, the linker is cleaved intracellularly (e.g., in the endosome or in the cytosol or in the nucleus or in another organelle).

In some embodiments, the payload moiety comprises a therapeutic moiety. In some such embodiments, the therapeutic moiety comprises a therapeutic nucleic acid (e.g., an siRNA, antisense nucleic acid, gene for gene therapy, immunostimulatory oligonucleotide, etc). In some such embodiments, the payload moiety comprises a therapeutic protein or protein complex including, for example, and antibody (or fragment or alternative format thereof). In some embodiments, the payload moiety comprises a small molecule therapeutic. In some embodiments, the payload moiety comprises a sugar and/or a lipid.

In some embodiments, the payload moiety comprises a detectable moiety. In some embodiments, the payload moiety comprises a targeting moiety. In some embodiments, the payload moiety comprises a coating moiety (e.g., a polymer such as polyethylene glycol [PEG]).

In some embodiments, a single oligonucleotide comprising a ligand sequence is associated with a plurality of payload moieties. In some embodiments, a single nanoparticle structure carries a plurality of different payload moieties.

In some embodiments, the payload moiety is attached to a ligand. In some embodiments, the ligand and payload moiety are attached to the nanoparticle through hybridization. In some embodiments, the payload moiety is delivered to a cellular target. In some embodiments, the payload moiety is releasable delivered to the cellular target. In some embodiments, the payload moiety is released from the ligand. In some embodiments, the payload is released as a moiety still attached to the ligand. In some embodiments, the moiety is cleaved from the ligand post, prior to or during release.

It will be appreciate by those skilled in the art, that in some embodiments, the total number of targeting moieties used for a delivery system is empirically determined, so that the number of target moieties, does not saturate and/or exceed the total number of reciprocal cellular receptors. In some embodiments, this is achieved by controlling the number and/or special arrangement of the targeting moieties attached to the three-dimensional nanoparticle structure. In some embodiments, this is achieved by controlling the number and/or special arrangement of the targeting moieties attached to the higher-ordered nanoparticle structure.

One feature of provided three-dimensional structures formed by assembly of oligonucleotides is that size and/or character of the structures, including number, type, and/or arrangement of ligand hybridization elements, can be designed through selection of oligonucleotide sequences according to known techniques. In some embodiments, delivery systems are prepared having at least two types of hybridization elements, in predetermined ratio(s) to one another, such that relative amounts of payload moieties to be hybridized to the delivery system are controlled.

In some embodiments, oligonucleotides are designed and/or selected to assemble into structures in which different payloads will hybridize to different arms within, and/or on different faces of an assembled structure.

Therapeutic Moieties

In some embodiments, therapeutic moieties are or comprise nucleic acid molecules. To give but a few examples, in some embodiments therapeutic moieties are or comprise DNA (e.g., antisense DNA) or RNA. In some embodiments, therapeutic moieties are or comprise siRNA, miRNA, or other small RNA whose presence correlates with altered level and/or activity of a gene product (protein or nucleic acid) of interest. In some embodiments, therapeutic moieties are or comprise genes (e.g., for gene therapy), with or without introns and/or regulatory elements.

In certain embodiments, a payload moiety is a drug selected from analgesics, anti-inflammatory agents, antihelminthics, anti-arrhythmic agents, anti-bacterial agents, anti-viral agents, anti-coagulants, anti-depressants, anti-diabetics, anti-epileptics, anti-fungal agents, anti-gout agents, anti-hypertensive agents, anti-malarials, anti-migraine agents, anti-muscarinic agents, anti-neoplastic agents, erectile dysfunction improvement agents, immunosuppressants, anti-protozoal agents, anti-thyroid agents, anxiolytic agents, sedatives, hypnotics, neuroleptics, β-blockers, cardiac inotropic agents, corticosteroids, diuretics, anti-parkinsonian agents, gastro-intestinal agents, histamine receptor antagonists, keratolyptics, lipid regulating agents, anti-anginal agents, Cox-2 inhibitors, leukotriene inhibitors, macrolides, muscle relaxants, nutritional agents, opiod analgesics, protease inhibitors, sex hormones, stimulants, muscle relaxants, anti-osteoporosis agents, anti-obesity agents, cognition enhancers, anti-urinary incontinence agents, anti-benign prostate hypertrophy agents, essential fatty acids, non-essential fatty acids, and mixtures thereof.

In some embodiments, a payload moiety is a drug selected from any one or more of acetretin, albendazole, albuterol, aminoglutethimide, amiodarone, amlodipine, amphetamine, amphotericin B, atorvastatin, atovaquone, azithromycin, baclofen, beclomethasone, benezepril, benzonatate, betamethasone, bicalutanide, budesonide, bupropion, busulfan, butenafine, calcifediol, calcipotriene, calcitriol, camptothecin, candesartan, capsaicin, carbamezepine, carotenes, celecoxib, cerivastatin, cetirizine, chlorpheniramine, cholecalciferol, cilostazol, cimetidine, cinnarizine, ciprofloxacin, cisapride, clarithromycin, clemastine, clomiphene, clomipramine, clopidogrel, codeine, coenzyme Q10, cyclobenzaprine, cyclosporin, danazol, dantrolene, dexchlorpheniramine, diclofenac, dicoumarol, digoxin, dehydroepiandrosterone, dihydroergotamine, dihydrotachysterol, dirithromycin, donezepil, efavirenz, eprosartan, ergocalciferol, ergotamine, essential fatty acid sources, etodolac, etoposide, famotidine, fenofibrate, fentanyl, fexofenadine, finasteride, fluconazole, flurbiprofen, fluvastatin, fosphenytoin, frovatriptan, furazolidone, gabapentin, gemfibrozil, glibenclamide, glipizide, glyburide, glimepiride, griseofulvin, halofantrine, ibuprofen, irbesartan, irinotecan, isosorbide dinitrate, isotretinoin, itraconazole, ivermectin, ketoconazole, ketorolac, lamotrigine, lansoprazole, leflunomide, lisinopril, loperamide, loratadine, lovastatin, L-thryroxine, lutein, lycopene, medroxyprogesterone, mifepristone, mefloquine, megestrol acetate, methadone, methoxsalen, metronidazole, miconazole, midazolam, miglitol, minoxidil, mitoxantrone, montelukast, nabumetone, nalbuphine, naratriptan, nelfinavir, nifedipine, nilsolidipine, nilutanide, nitrofurantoin, nizatidine, omeprazole, oprevelkin, oestradiol, oxaprozin, paclitaxel, paracalcitol, paroxetine, pentazocine, pioglitazone, pizofetin, pravastatin, prednisolone, probucol, progesterone, pseudoephedrine, pyridostigmine, rabeprazole, raloxifene, rofecoxib, repaglinide, rifabutine, rifapentine, rimexolone, ritanovir, rizatriptan, rosiglitazone, saquinavir, sertraline, sibutramine, sildenafil citrate, simvastatin, sirolimus, spironolactone, sumatriptan, tacrine, tacrolimus, tamoxifen, tamsulosin, targretin, tazarotene, telmisartan, teniposide, terbinafine, terazosin, tetrahydrocannabinol, tiagabine, ticlopidine, tirofibran, tizanidine, topiramate, topotecan, toremitfene, tramadol, tretinoin, troglitazone, trovafloxacin, ubidecarenone, valsartan, venlafaxine, verteporfin, vigabatrin, vitamin A, vitamin D, vitamin E, vitamin K, zafirlukast, zileuton, zolmitriptan, zolpidem, zopiclone, pharmaceutically acceptable salts, isomers, and derivatives thereof, and mixtures thereof.

In some embodiments, a payload moiety is a drug selected from an antiproliferative or chemotherapeutic drug. In certain embodiments, the hydrophobic antiproliferative or chemotherapeutic drug is selected from any one or more of a taxane (e.g., paclitaxel), vincristine, adriamycin, vinca alkaloids (e.g., vinblastine), anthracyclines (e.g., doxorubicin), epipodophyllotoxins (e.g., etoposide), cisplatin, methotrexate, actinomycin D, actinomycin D, dolastatin 10, colchicine, emetine, trimetrexate, metoprine, cyclosporine, daunorubicin, teniposide, amphotericin, alkylating agents (e.g., chlorambucil), 5-fluorouracil, campthothecin, cisplatin, and metronidazole.

In some embodiments, a payload moiety is a drug selected from Abarelix, aldesleukin, Aldesleukin, Alemtuzumab, Alitretinoin, Allopurinol, Altretamine, Amifostine, Anastrozole, Arsenic trioxide, Asparaginase, Azacitidine, BCG Live, Bevacuzimab, Avastin, Fluorouracil, Bexarotene, Bleomycin, Bortezomib, Busulfan, Calusterone, Capecitabine, Camptothecin, Carboplatin, Carmustine, Celecoxib, Cetuximab, Chlorambucil, Cisplatin, Cladribine, Clofarabine, Cyclophosphamide, Cytarabine, Dactinomycin, Darbepoetin alfa, Daunorubicin, Denileukin, Dexrazoxane, Docetaxel, Doxorubicin (neutral), Doxorubicin hydrochloride, Dromostanolone Propionate, Epirubicin, Epoetin alfa, Erlotinib, Estramustine, Etoposide Phosphate, Etoposide, Exemestane, Filgrastim, floxuridine fludarabine, Fulvestrant, Gefitinib, Gemcitabine, Gemtuzumab, Goserelin Acetate, Histrelin Acetate, Hydroxyurea, Ibritumomab, Idarubicin, Ifosfamide, Imatinib Mesylate, Interferon Alfa-2a, Interferon Alfa-2b, Irinotecan, Lenalidomide, Letrozole, Leucovorin, Leuprolide Acetate, Levamisole, Lomustine, Megestrol Acetate, Melphalan, Mercaptopurine, 6-MP, Mesna, Methotrexate, Methoxsalen, Mitomycin C, Mitotane, Mitoxantrone, Nandrolone, Nelarabine, Nofetumomab, Oprelvekin, Oxaliplatin, Paclitaxel, Palifermin, Pamidronate, Pegademase, Pegaspargase, Pegfilgrastim, Pemetrexed Disodium, Pentostatin, Pipobroman, Plicamycin, Porfimer Sodium, Procarbazine, Quinacrine, Rasburicase, Rituximab, Sargramostim, Sorafenib, Streptozocin, Sunitinib Maleate, Talc, Tamoxifen, Temozolomide, Teniposide, VM-26, Testolactone, Thioguanine, 6-TG, Thiotepa, Topotecan, Toremifene, Tositumomab, Trastuzumab, Tretinoin, ATRA, Uracil Mustard, Valrubicin, Vinblastine, Vincristine, Vinorelbine, Zoledronate, or Zoledronic acid.

In some embodiments, a payload moiety is a drug for the treatment for Alzheimer's Disease such as Aricept® or Excelon®; a drug for the treatment for Parkinson's Disease such as L-DOPA/carbidopa, entacapone, ropinrole, pramipexole, bromocriptine, pergolide, trihexephendyl, or amantadine; an agent for treating Multiple Sclerosis (MS) such as beta interferon (e.g., Avonex® and Rebif®), Copaxone®, or mitoxantrone; a drug for the treatment for asthma such as a steroid, albuterol or Singulair®; an agent for treating schizophrenia such as zyprexa, risperdal, seroquel, or haloperidol; an anti-inflammatory agent such as corticosteroids, TNF blockers, IL-1 RA, azathioprine, cyclophosphamide, or sulfasalazine; an immunomodulatory and immunosuppressive agent such as cyclosporin, tacrolimus, rapamycin, mycophenolate mofetil, interferons, corticosteroids, cyclophophamide, azathioprine, or sulfasalazine; a neurotrophic factor such as acetylcholinesterase inhibitors, MAO inhibitors, interferons, anti-convulsants, ion channel blockers, riluzole, or anti-Parkinsonian agents; an agent for treating cardiovascular disease such as beta-blockers, ACE inhibitors, diuretics, nitrates, calcium channel blockers, or statins; an agent for treating liver disease such as corticosteroids, cholestyramine, interferons, or anti-viral agents; an agent for treating blood disorders such as corticosteroids, anti-leukemic agents, or growth factors; and an agent for treating immunodeficiency disorders such as gamma globulin.

In some embodiments, a payload moiety is a DNA plasmid, oligonucleotides, short interfering RNA (siRNAs), micro RNA (miRNAs), short hairpin RNA (shRNAs), antisense RNA, and/or other RNA-based therapeutics. Other ionic, or charged, therapeutic agents include oligopeptides, peptides, monoclonal antibodies, cytokines, and other protein therapeutics.

One of ordinary skill in the art will understand that in any of the embodiments provided above, one or more compounds (e.g., drugs) may be used in combination as a payload.

Detectable Moieties

In some embodiments, detectable moieties are or comprise primary labels and/or secondary labels as defined and described above and herein. For example, in some embodiments, detectable moieties are primary labels, such as radioisotopes (e.g., ³²P, ³³P, ³⁵S, or ¹⁴C) and/or mass-tags. In some embodiments, detectable moieties are fluorescent labels and/or signal generating reporter groups which can be detected without further modifications. In some embodiments, detectable moieties are fluorescent labels and/or dyes, and/or fluorophores, as defined and described herein.

In some embodiments, detectable moieties are or comprise a diagnostic label. Exemplary diagnostic labels include, for instance, radionuclides, fluorophores, chromophores, paramagnetic ions or moieties, superparamagnetic nanoparticles, heavy metal ions, enzymes, biotin, etc. In some embodiments, a diagnostic label is detected in vivo or in vitro using, e.g., radioactivity measurement, gamma scintography, positron emission tomography, nuclear magnetic resonance spectroscopy, magnetic resonance imaging, fluorescence spectroscopy, photoimaging, x-ray tomography, electron microscopy, enzyme assay, and the like.

Targeting Moieties

In some embodiments, targeting moieties target cell surface markers. In some embodiments, targeting moieties target intracellular markers. In some embodiments, targeting moieties are or comprise cell penetrating entities (e.g., that facilitate entry of targeting delivery systems into cells).

In some embodiments, as noted herein, provided delivery systems comprise a plurality of delivery structures (e.g., oligonucleotide delivery structures) associated with one another to form an assemblage. In some such embodiments, some or all of the structures in such an assemblage contain or are associated with targeting moieties. In some embodiments, delivery structures at the surface of the assemblage are associated with targeting moieties. In some embodiments, other delivery structures (e.g., structures internal to the assemblage) are not associated with targeting moieties.

In some embodiments, targeted markers are selected from the group consisting of polysaccharides, polypeptides, carbohydrates and lipids. It is to be understood that the terms “proteins,” “polysaccharides,” “polypeptides,” “carbohydrates,” and “lipids” are intended to also refer to related compounds, or derivatives, such as glycoproteins, glycolipids, lipopolysaccharides, proteoglycans, lipoproteins, lipid-protein complexes, nucleosomes, and lipoteichoic acids.

To give but a few examples, in some embodiments, a targeted marker can be a cell surface receptor such as a transmembrane receptor (e.g., G-protein coupled receptors, tyrosine kinase receptors, growth factor receptors). In some embodiments, a targeted marker comprises a molecule or entities that is found expressed exclusively or predominantly on, in, or in association with pathological cells, tissues or organs. For example, a targeted marker may be or comprise one or more of asialofetuin receptors (hepatocytes), viral antigens (cells infected by herpes or other viruses), scavenger receptors (macrophages), HER-2/neu (some breast cancer types), cytokine receptors, receptors of growth factors, surface glycolipids and glycoproteins, integrins, selectins, etc. For example, malignant epithelial cells in primary human lung carcinomas coexpress in vivo PDGF (ligand) and PDGF receptor (cell or tissue marker) (Antoniades, H. N., et al., Proceedings of the National Academy of Sciences, 89:3942-6 (1992), the teachings of which are hereby incorporated by reference in their entirety); macrophage colony-stimulating factor (ligand) and its receptor (cell or tissue marker) are expressed in ovarian and endometrial carcinomas (Baiocchi, G., et al., Cancer, 67:990-6 (1991), the teachings of which are hereby incorporated by reference in their entirety); coexpression of HER-2/neu and the epidermal growth factor receptor (cell or tissue marker) has been observed in 65% of epithelial ovarian cancers and in a limited number of normal tissue from a fraction of donors (Bast, R. C., Jr., et al., Cancer, 71:1597-601 (1993), the teachings of which are hereby incorporated by reference in their entirety); cellular expression of Fuc-GMI generally was seen together with NCAM in lung carcinomas (Brezicka, F. T., et al., Tumour Biology, 13:308-15 (1992), the teachings of which are hereby incorporated by reference in their entirety); thrombospondin-1 is codistributed with CD51 in most of the invasive lobular breast carcinoma cells (40 to 80%) and with CD36 in a subpopulation (30 to 40%) of these cells (Clezardin, P., et al., Cancer Research, 53:1421-30 (1993), the teachings of which are hereby incorporated by reference in their entirety); both H-2 and Le(y) were coexpressed in the same individual colorectal carcinoma cells in 92% of cancers expressing both these blood group antigens (Cooper, H. S., et al., Am. J. Pathol., 138:103-10 (1991), the teachings of which are hereby incorporated by reference in their entirety); uPAR and plasminogen activator inhibitor-1 were overexpressed in invasive breast cancer in comparison with normal and benign breast tissues (Costantini, V., et al., Cancer, 77:1079-88 (1996), the teachings of which are hereby incorporated by reference in their entirety); coexpression of at least two of cytokeratin, neurofilament, vimentin, and desmin was found in pulmonary neoplasms, whereas in normal tissues these have a different and non-overlapping distribution (Gatter, K. C., et al., J. of Clinical Pathology, 39:950-4 (1986), the teachings of which are hereby incorporated by reference in their entirety); the majority of cases of childhood medulloblastoma expressed two or more receptor proteins of EGFR family members (EGFR, HER2, HER3, and HER4); coexpression of the HER2 and HER4 receptors occurred in 54% (Gilbertson, R. J., et al., Cancer Research, 57:3272-80 (1997), the teachings of which are hereby incorporated by reference in their entirety); coexpression of multiple (three or more) mucin core proteins occurred in 15 of 25 (60%) advanced (stages III and IV) cancers compared with 1 of 8 (12.5%) early (stages I and II) cancers in gastric adenocarcinomas (Ho, S. B., et al., Cancer Research, 55:2681-90 (1995), the teachings of which are hereby incorporated by reference in their entirety); in colorectal cancer, EGFR positive malignant tumors showed coexpression of IL-4 receptor (Kaklamanis, L., et al., Brit. J. of Cancer, 66:712-6 (1992), the teachings of which are hereby incorporated by reference in their entirety); overexpression of p53 protein correlated closely with the overexpression of c-erbB-2 in malignant salivary gland tumors (Kamio, N., et al., Virchows Archiv., 428:75-83 (1996); epidermal growth factor receptor EGF-R and C-erbB-2 have been shown to be expressed in human tumors and in some cases relate to the histological grade of the lesions and clinical outcome (Lakshmi, S., et al., Pathobiology, 65:163-8 (1997), the teachings of which are hereby incorporated by reference in their entirety); colocalization of MMP-9 was seen with high molecular weight melanoma-associated antigen, the pericyte marker, in ductal breast cancer (Nielsen, B. S., et al., Lab. Investigation, 77:345-55 (1997), the teachings of which are hereby incorporated by reference in their entirety); distribution of laminin-5-positive budding cancer cells at the invasion front in colon adenocarcinomas was identical to that of the receptor for urokinase-type plasminogen activator (Pyke, C., et al., Cancer Research, 55:4132-9 (1995), the teachings of which are hereby incorporated by reference in their entirety), etc.

Coating Moieties

In some particular embodiments, coating moieties are entities that protect or shield other components of provided delivery systems (e.g., the delivery structure itself or one or more payload moieties associated with the delivery structure).

In some embodiments, a coating moiety is or comprises a polymer. In some embodiments, a coating moiety is or is comprised of a biocompatible entity (e.g., a biocompatible polymer). In some embodiments, a coating moiety is or is comprised of a biodegradable entity (e.g., a biodegradable polymer). To give but a few specific examples, in some embodiments, a coating moiety is or comprises polyethylene glycol (PEG).

Those of ordinary skill in the art will readily appreciate that, in some embodiments, a single additional component may comprise a plurality of different payload moieties. To give but one example, in some embodiments, a provided therapeutic or diagnostic moiety further comprises a coating moiety (e.g., polyethylene glycol), for example to stabilize the therapeutic or diagnostic moiety.

Coatings

In some embodiments, provided delivery systems are completely or partially covered with a coating (e.g., a polymer coating) that is not a component of the delivery system (i.e., is not associated with the delivery structure by hybridization). A wide variety of coating systems is known in the art. For example, in some embodiments, provided delivery systems are embedded in or otherwise surrounded a coating.

In some embodiments, a coating comprises a polymer. In some embodiments, a coating comprises a hydrogel. In some embodiments a coating is biocompatible and/or biodegradable.

In some embodiments, provided delivery structures are coated with a coating having an average thickness in a range of about 1 nm and about 100 μm. In some embodiments, a coating has an average thickness in a range of about 1 μm and about 50 μm. In some embodiments, a coating has an average thickness in a range of about 2 μm and about 5 μm. In some embodiments, the average thickness of a coating is or more than about 1 nm, about 100 nm, about 500 nm, about 1 μm, about 2 μm, about 3 μm, about 4 μm, about 5 μm, about 10 μm, bout 20 μm, about 50 μm, about 100 μm. In some embodiments, a coating has an average thickness in a range of any two values above.

In some embodiments, a coating can be or comprise a decomposition film. Decomposition of such coating may be characterized by the substantially sequential degradation of at least a portion of the polyelectrolyte layers that make up the thin films. Degradation may be at least partially hydrolytic, at least partially enzymatic, at least partially thermal, and/or at least partially photolytic.

In some embodiments, a coating comprises multilayer units with alternating layers of opposite charge, such as alternating anionic and cationic layers. At least one of the layers in a coating includes a degradable polyelectrolyte. In some embodiments, a coating include a plurality of polyelectrolyte layers. In some embodiments, a coating include a plurality of a single unit (e.g., a bilayer unit, a tetralayer unit, etc.). In some embodiments, a coating is a composite that include more than one units. For example, more than one units can have be different in film materials (e.g., polymers), film architecture (e.g., bilayers, tetralayer, etc.), film thickness, and/or releasable agents that are associate with one of the units. In some embodiments, a coating is a composite that include more than one bilayer units, more than one tetralayer units, or any combination thereof. In some embodiments, a coating is a composite that include a plurality of a single bilayer unit and a plurality of a single tetralayer unit.

In some particular embodiments, provided delivery structures are coated with a degradable coating. In some such embodiments, the coating is hydrolytically degradable, biodegradable, thermally degradable, and photolytically degradable polyelectrolytes.

Various degradable (e.g., hydrolytically degradable) polymers known in the art include for example, certain polyesters, polyanhydrides, polyorthoesters, polyphosphazenes, and polyphosphoesters. Biodegradable polymers known in the art, include, for example, certain polyhydroxyacids, polypropylfumerates, polycaprolactones, polyamides, poly(amino acids), polyacetals, polyethers, biodegradable polycyanoacrylates, biodegradable polyurethanes and polysaccharides. Specific exemplary biodegradable polymers include but are not limited to polylysine, poly(lactic acid) (PLA), poly(glycolic acid) (PGA), poly(caprolactone) (PCL), poly(lactide-co-glycolide) (PLG), poly(lactide-co-caprolactone) (PLC), and poly(glycolide-co-caprolactone) (PGC). Those skilled in the art will appreciate that co-polymers, mixtures, and adducts of these polymers may also be employed.

In some embodiments, degradable polymers comprise polyelectrolytes, for example with anionic and/or cationic groups distributed along a polymer backbone (e.g., pendant from or incorporated into the backbone). Exemplary such anionic groups include, for example, carboxylate, sulfonate, sulphate, phosphate, nitrate, or other negatively charged or ionizable groupings. Exemplary such cationic groups include, for example, protonated amine, quaternary ammonium or phosphonium-derived functions or other positively charged or ionizable groups, may be disposed in side groups pendant from the backbone, may be attached to the backbone directly, or can be incorporated in the backbone itself.

A variety of hydrolytically degradable amine containing polyesters bearing cationic side chains have been developed (Putnam et al. Macromolecules 32:3658-3662, 1999; Barrera et al. J. Am. Chem. Soc. 115:11010-11011, 1993; Kwon et al. Macromolecules 22:3250-3255, 1989; Lim et al. J. Am. Chem. Soc. 121:5633-5639, 1999; Zhou et al. Macromolecules 23:3399-3406, 1990; each of which is incorporated herein by reference). Examples of these polyesters include poly(L-lactide-co-L-lysine) (Barrera et al. J. Am. Chem. Soc. 115:11010-11011, 1993; incorporated herein by reference), poly(serine ester) (Zhou et al. Macromolecules 23:3399-3406, 1990; which is incorporated herein by reference), poly(4-hydroxy-L-proline ester) (Putnam et al. Macromolecules 32:3658-3662, 1999; Lim et al. J. Am. Chem. Soc. 121:5633-5639, 1999; each of which is incorporated herein by reference), and more recently, poly[α-(4-aminobutyl)-L-glycolic acid].

In addition, poly(β-amino ester)s, prepared from the conjugate addition of primary or secondary amines to diacrylates, are suitable for use. Typically, poly(β-amino ester)s have one or more tertiary amines in the backbone of the polymer, preferably one or two per repeating backbone unit. Alternatively, a co-polymer may be used in which one of the components is a poly(β-amino ester). Poly(β-amino ester)s are described in U.S. Pat. Nos. 6,998,115 and 7,427,394, entitled “Biodegradable poly(β-amino esters) and uses thereof” and Lynn et al., J. Am. Chem. Soc. 122:10761-10768, 2000, the entire contents of both of which are incorporated herein by reference.

In some embodiments, poly(β-amino ester)s can have a formula below:

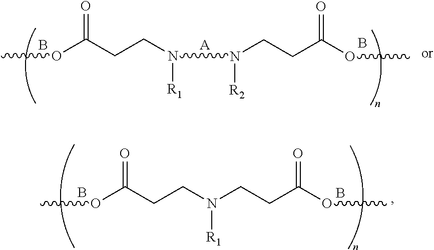

where A and B are linkers which may be any substituted or unsubstituted, branched or unbranched chain of carbon atoms or heteroatoms. The molecular weights of the polymers may range from 1000 g/mol to 20,000 g/mol, preferably from 5000 g/mol to 15,000 g/mol. In certain embodiments, B is an alkyl chain of one to twelve carbons atoms. In other embodiments, B is a heteroaliphatic chain containing a total of one to twelve carbon atoms and heteroatoms. The groups R₁ and R₂ may be any of a wide variety of substituents. In certain embodiments, R₁ and R₂ may contain primary amines, secondary amines, tertiary amines, hydroxyl groups, and alkoxy groups. In certain embodiments, the polymers are amine-terminated; and in other embodiments, the polymers are acrylated terminated. In some embodiments, the groups R₁ and/or R₂ form cyclic structures with the linker A.

Exemplary poly(β-amino esters) include

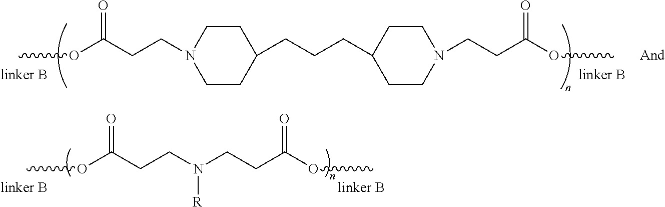

Exemplary R groups include hydrogen, branched and unbranched alkyl, branched and unbranched alkenyl, branched and unbranched alkynyl, aryl, halogen, hydroxyl, alkoxy, carbamoyl, carboxyl ester, carbonyldioxyl, amide, thiohydroxyl, alkylthioether, amino, alkylamino, dialkylamino, trialkylamino, cyano, ureido, a substituted alkanoyl group, cyclic, cyclic aromatic, heterocyclic, and aromatic heterocyclic groups, each of which may be substituted with at least one substituent selected from the group consisting of branched and unbranched alkyl, branched and unbranched alkenyl, branched and unbranched alkynyl, amino, alkylamino, dialkylamino, trialkylamino, aryl, ureido, heterocyclic, aromatic heterocyclic, cyclic, aromatic cyclic, halogen, hydroxyl, alkoxy, cyano, amide, carbamoyl, carboxylic acid, ester, carbonyl, carbonyldioxyl, alkylthioether, and thiol groups.

Exemplary linker groups B includes carbon chains of 1 to 30 carbon atoms, heteroatom-containing carbon chains of 1 to 30 atoms, and carbon chains and heteroatom-containing carbon chains with at least one substituent selected from the group consisting of branched and unbranched alkyl, branched and unbranched alkenyl, branched and unbranched alkynyl, amino, alkylamino, dialkylamino, trialkylamino, aryl, ureido, heterocyclic, aromatic heterocyclic, cyclic, aromatic cyclic, halogen, hydroxyl, alkoxy, cyano, amide, carbamoyl, carboxylic acid, ester, carbonyl, carbonyldioxyl, alkylthioether, and thiol groups. Such a polymer may include, for example, between 5 and 10,000 repeat units.

In some embodiments, the poly(β-amino ester)s are selected from the group consisting of

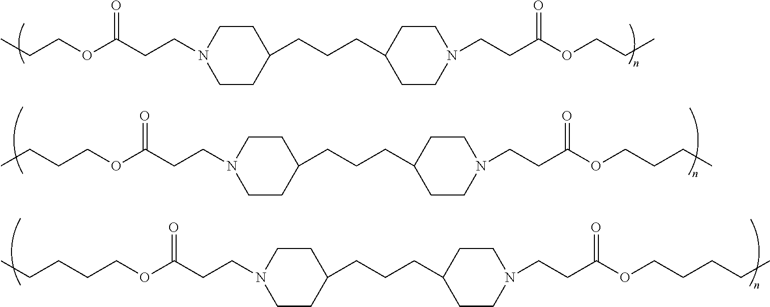

derivatives thereof, and combinations thereof

Alternatively or additionally, zwitterionic polyelectrolytes may be used. Such polyelectrolytes may have both anionic and cationic groups incorporated into the backbone or covalently attached to the backbone as part of a pendant group. Such polymers may be neutrally charged at one pH, positively charged at another pH, and negatively charged at a third pH. For example, a coating may be constructed by layer-by-layer (LbL) deposition using dip coating in solutions of a first pH at which one layer is anionic and a second layer is cationic. If such a coating is put into a solution having a second different pH, then the first layer may be rendered cationic while the second layer is rendered anionic, thereby changing the charges on those layers.

In some embodiments, a coating may include a mixture of degradable and non-degradable polyelectrolytes. Any non-degradable polyelectrolyte can be used. Exemplary non-degradable polyelectrolytes that could be used in a coating include poly(styrene sulfonate) (SPS), poly(acrylic acid) (PAA), linear poly(ethylene imine) (LPEI), poly(diallyldimethyl ammonium chloride) (PDAC), and poly(allylamine hydrochloride) (PAH).

Uses

Those of ordinary skill in the art will appreciate that provided delivery systems are useful in any of a variety of contexts. In many embodiments, provided delivery systems are useful in medicine, for example to deliver therapeutic and/or detectable moieties. In some embodiments, provided delivery systems achieve targeted delivery of therapeutic and/or detectable moieties, for example to sites on or within cells.

### EXEMPLIFICATION

### Example 1

### Self-Assembling Nanoparticle Delivery System, for Example, for siRNAs

The present Example describes certain particular embodiments of a delivery system in accordance with the present invention. Specifically, the present Example describes molecularly identical, self-assembling nanoparticles for targeted gene silencing in tumor-bearing mice, and particularly describes such nanoparticles and their assembly and use without using any cationic delivery agents. Described nanoparticles are useful, among other things, in the delivery of small interfering RNAs (siRNAs). siRNAs, and the RNA interference (RNAi) pathways in which they are involved, have the potential to address a broad range of diseases, including, for example, cancer.¹⁻³

Monodisperse nanoparticles were prepared with defined molecular structure through the self-assembly of DNA/RNA nucleic acids. External surfaces of these nanoparticles were specifically modified at different positions with affinity ligands, including peptides and folate. Interestingly, potent delivery activity was observed for nanoparticles with folate alone, in the absence of known endosomal escape moieties. Optimal delivery was observed with at least three folate ligands per nanoparticle, preferably having a defined spatial orientation on the particle.

In vivo, provided nanoparticles achieved targeted delivery, resulting in effective RNAi in tumor xeno-grafts. The ability to precisely control the molecular composition and orientation of ligands on such nanoparticles offers broad potential for their use as therapeutic nucleic acid delivery carriers.

### Results

The discovery of RNA interference (RNAi) in mammalian cells has attracted broad therapeutic interest, given its potential to induce specific reduction of target transcripts.^{1,2} However, safe and effective delivery methods are needed in order for the therapeutic potential of RNAi to be realized.³ To date, a variety of materials have been investigated for their potential to deliver siRNA as nanoformulations.⁴ Nanoparticulate therapeutics of a size above 20 nm have the potential to avoid renal clearance, which is a typical occurrence for monomeric siRNA, and to enhance delivery to certain tumor types through the enhanced permeability and retention effect (EPR).⁵

Many strategies for delivery of siRNA utilize cationic carriers, for example to “hide” the nucleic acid charge so that trans-membrane delivery is facilitated. Although animal studies have shown siRNA-mediated gene silencing in tumor models through systemic administration utilizing such cationic carriers, the inherent toxicity and lack of tissue specificity of the cationic carriers have remained major challenges.^{6,7} In addition, these delivery formulations are generally polydisperse when formulated, creating a distribution of particles with different sizes and chemistries.

The present disclosure provides improved nanoparticle delivery systems, constructed from oligonucleotide elements that hybridize with one another to form stable nanoscale structures comprising a plurality of double-stranded “arms” and at least one single-stranded ligands hybridization site. The present Example describes particular embodiments of such nanoparticle delivery systems, and specifically describes a collection of nanoparticle devices that are molecularly identical to one another and achieve delivery of selected payloads, linked to nucleic acid ligands that hybridize to ligand hybridization sites within the nanoparticle.

The present Example specifically describes delivery of siRNA payloads, and particularly of double-stranded siRNAs that have a single stranded overhang that acts as the ligands and hybridizes to ligand hybridization sites in the nanoparticle.

The particular embodiment of a provided nanoparticle delivery system described in the present Example is an oligo-nucleic acid nanoparticle (ONP) formed from six oligonucleotide strands, with sticky-overhangs at the 3′ end, that self-assemble into a tetrahedron consisting of 186 Watson-Crick base pairs.⁸ The six edges are each 30 base pairs in length and the theoretical tetrahedron height is 8 nm with 10 nm edges. Each edge contains a nick with a sticky-overhang in the middle where the 5′ and 3′ ends of an oligonucleotide meet. The sticky-overhang is designed to have sequence specific hybridization with the complementary overhang of an siRNA (total of 6 siRNAs per nanoparticle, 1 per edge) (FIG. 17).

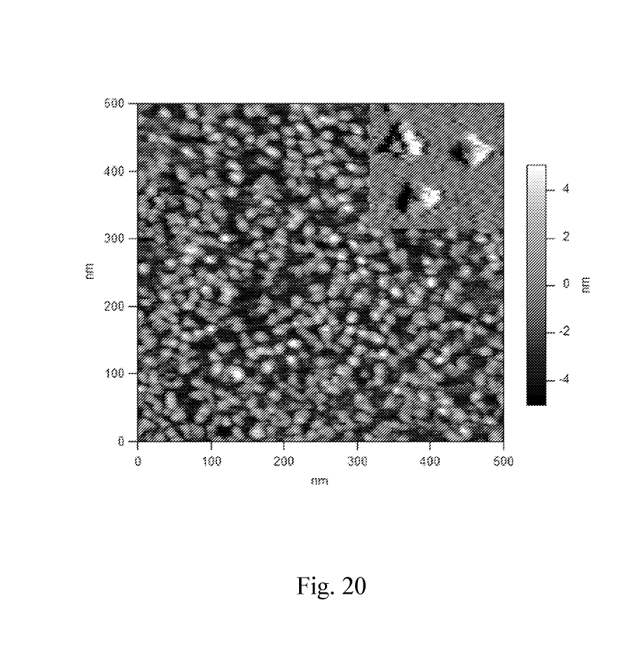

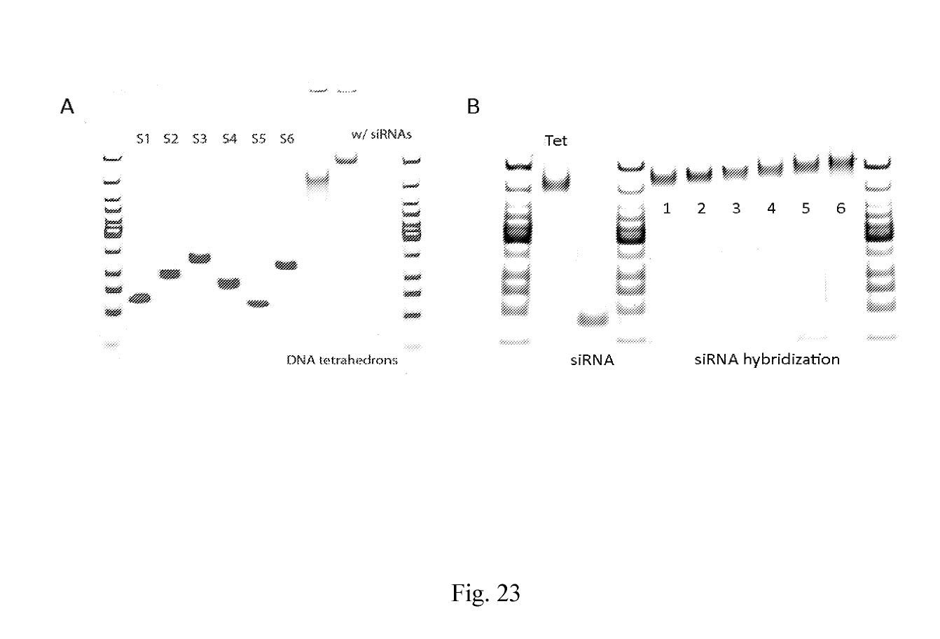

The utilized siRNA payloads utilized in the present Example were constructed with chemically modified ribonucleotides shown to significantly enhance serum stability as well as reduce immune stimulation potential.⁹ Hybridization of siRNAs to the core DNA tetrahedron was confirmed by native PAGE analysis and the yield was over 98% (FIG. 23). The tetrahedron structure was verified by AFM imaging in aqueous buffer solution (FIG. 20). High-resolution images confirmed the presence of the three upper edges of individual tetrahedrons as well as its height of ˜7.5 nm. The tetrahedron size by DLS measurement showed a hydrodynamic diameter of ˜30 nm with a narrow size distribution.

It has been suggested that the optimal particle size of a nano-delivery carrier is 10-100 nm.^{10,11} In theory, the ONPs described in this Example are large enough to avoid renal filtration (>20 nm) but small enough to penetrate through the leaky vasculatures in a tumor region, bind to cell surface receptors, and facilitate intracellular uptake, while reducing reticuloendothelial system (RES)-mediated clearance. Because the ONPs described herein are molecularly defined, they exist as a single uniform population in terms of size and shape. This is distinctly different from traditional cationic delivery carriers that can exist in a range of undefined shapes and sizes. The nucleic acid composition of provided ONPs renders them addressable at any location by the design of overhangs allowing precise spatial control of all decorating ligands via hybridization.

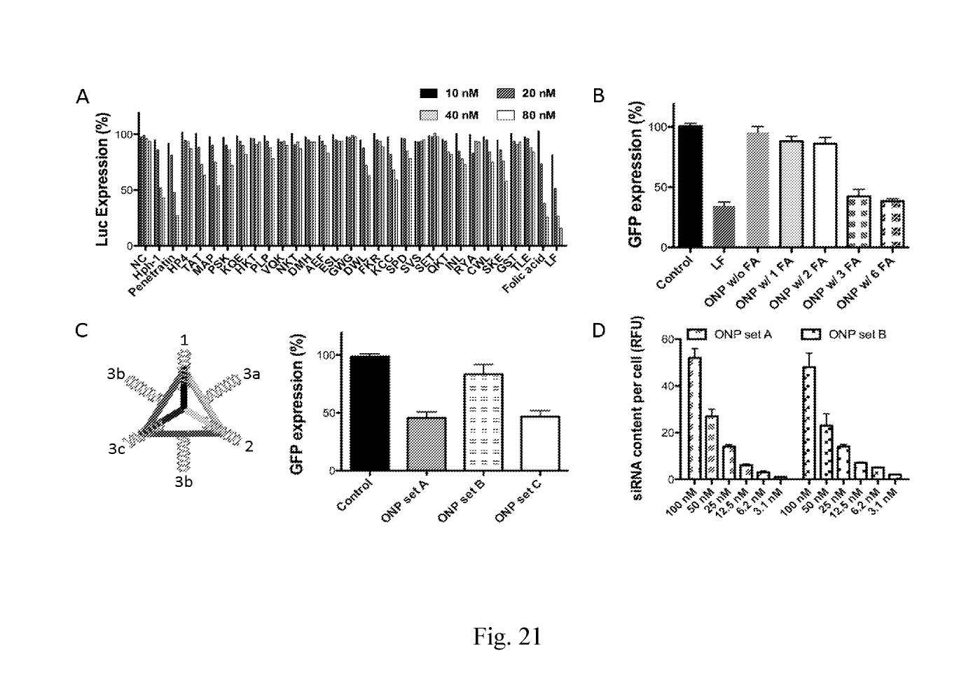

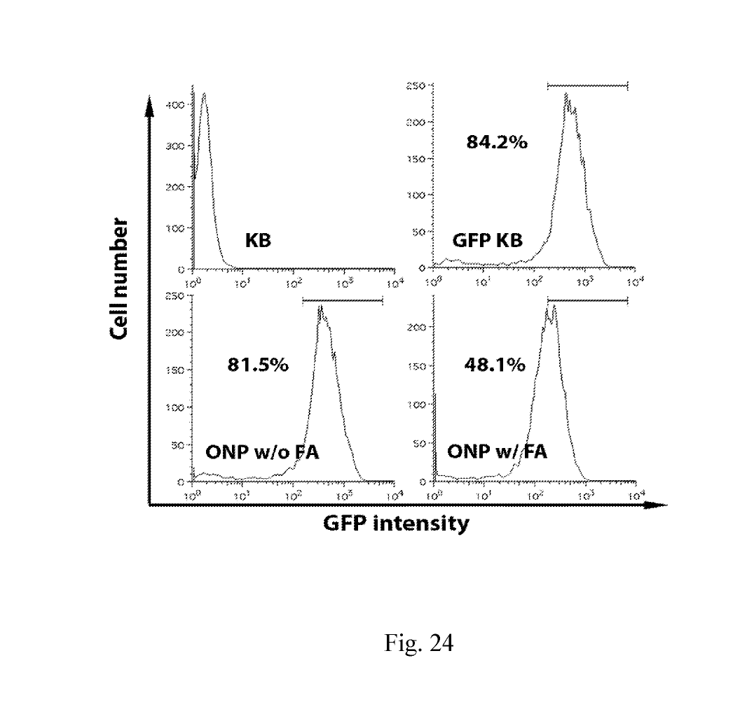

To ascertain whether the ONPs described herein provide effective targeted delivery of siRNA to human cancer cells, we investigated various cancer targeting ligands from peptides to small molecules. Without wishing to be bound by any particular theory, it is proposed that intracellular delivery of ONPs described herein may be further promoted by active targeting of specific surface receptors on cancer cells, as has been suggested for other delivery systems.^{12,13} Among the 30 different targeting moieties tested here, we found that one particular targeting agent, folic acid, conjugated to the ONPs exhibited surprisingly potent gene silencing, achieving over 50% reduction of firefly luciferase expression in Hela cells in a dose dependent manner (FIG. 21A). A few cationic peptides (Hph-1 and Penetratin) also reduced firefly luciferase expression, however the structural integrity of the ONPs became obscure due to the charge interaction between the cationic peptides and anionic nucleic acids. Folate-mediated gene silencing was also confirmed in KB cells (GFP-KB). At a dose of 35 nM, we observed over 60% reduction of GFP expression in these cells (FIG. 24).

In addition to optimal particle size and cancer specificity for intracellular siRNA delivery, provided ONPs allow full control of spatial orientation of targeting moieties as well as the density of such targeting moieties on the nanoparticle surface. Since the geometry of the ONPs is well defined, it is possible to investigate structure/function correlation of targeting moiety density and orientation for optimal gene silencing.

The present disclosure also surprisingly demonstrates that, in some embodiments, spatial orientation and/or density of a targeting moiety (e.g., folate) dramatically impacts effective delivery. Specifically, six different over-hang sequences were designed for siRNA hybridization, and several parameters were varied: 1) the number of hybridized siRNAs; 2) the density of targeting folate moieties; and 3) the location/spatial orientation of targeting folate moieties. We can specifically control the number of siRNAs on the core DNA tetrahedron from one to six hybridized siRNA (FIG. 23). By using both folic acid conjugated and non-conjugated siRNAs, the level of GFP gene silencing was investigated by varying the number of folate moieties, while maintaining the same number of siRNA on each nanoparticle (FIG. 21B).

Our results indicate that GFP gene silencing is observed when at least three folate moieties are present. Interestingly, more than three folates on one particle (e.g., four, five, and six folate moieties) did not improve gene silencing efficiency in these studies.

Our results also demonstrated that orientation/location of the targeting folate moieties did affect the gene silencing (FIG. 21C). When one plane of the tetrahedron was decorated with folate (three folic acid on same plane (or face): example location 1, 2, 3 or 1, 2, 6 in (FIG. 21C), GFP silencing was observed. However, when three folic acids were decorated onto different planes/faces (example location 1, 2, 4 or 1, 2, 5) of a tetrahedron, we did not observe any GFP silencing. Interestingly, our confocal study revealed that the intracellular uptake of these nanoparticles was similar, regardless of folic acid location; however, potent gene silencing was only observed for nanoparticles with the appropriate spatial orientation of folate (FIG. 21D). Conventional nanoparticles do not permit precise control of ligand density and orientation on each single nanoparticle, so that prior to the present invention it has been difficult or impossible to assess what impact density and/or location of ligands can have on nanoparticle/cell membrane interactions and/or intracellular uptake pathways.^{14,15} To our knowledge, the work described in the present Example is the first in vitro evaluation of ways in which ligand density and orientation can alter the function of a nanoparticle-delivery system.

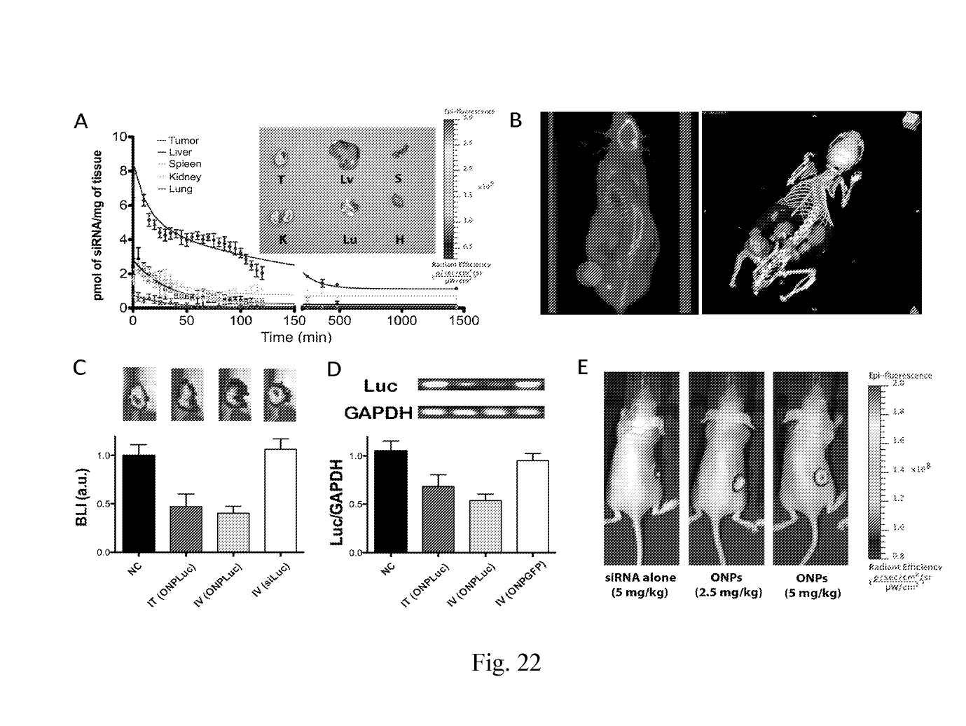

To verify in vivo delivery of ONPs, a pharmacokinetic (PK) profile and organ bio-distribution were investigated in nude mice bearing KB xenograft tumors. Cy5 labeled ONPs with folate ligands (3 nmol of siRNA, ˜2.5 mg/kg) were systemically delivered via tail-vein injection and their in vivo behavior was quantitatively measured for 24 h by hybrid fluorescence molecular tomography fused with computed tomography (FMT-CT).^{16,17} Co-registration of FMT and CT has provided high-resolution fluorescence images for the tumor targeting by ONPs as well as the PK of nanoparticles in five major organs. As shown in FIG. 22A, ONPs primarily accumulate in tumor and kidney, with little accumulation in other organs such as liver, spleen, lung, and heart. Ex vivo fluorescence images at 12 h post-injection also correspond well with the PK data. A reconstructed 3D FMT-CT image of a tumor bearing mouse supports the accumulation of ONPs in the tumor region as early as 25 min post-injection (FIG. 22B). In addition, the blood half-life data indicate that ONPs have a longer blood circulation time (t_{1/2}˜24 2 min) compared to native siRNA (t½˜6 min) (FIG. 22).¹⁸

In order to assess the therapeutic potential of ONPs as cationic-free gene delivery carriers, we conducted in vivo gene silencing of firefly luciferase expressing KB xenograft. Folic acid conjugated ONPs with anti-Luc siRNA were administrated (dose: 2.5 mg/kg siRNA) into mice either by tail-vein injection or intra-tumor injection. Silencing was tested 48 h post-injection by measuring bioluminescent intensity in the tumor. FIG. 22C exhibits the percent expression of bioluminescence in the tumor. Both tail vein and intra-tumor injections resulted in approximately 60% decrease in bioluminescent intensity. Comparatively, when mice were injected with naked folic acid conjugated anti-Luc siRNAs (not delivered by self-assembled nanoparticles), no decrease in bioluminescent intensity was observed. This result corresponds well with our in vitro silencing experiment, confirming that the ONPs are required to achieve gene silencing. In addition, measurement of the firefly luciferase mRNA level strongly supports the target specific mRNA cleavage by both systemic and local delivery of ONPs (FIG. 22D).

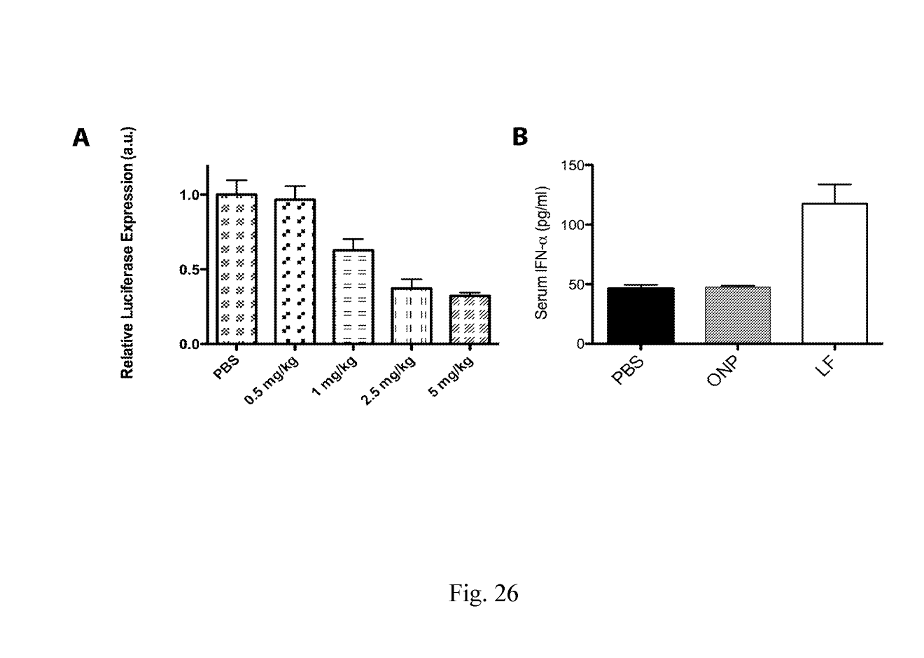

A dose dependent study revealed that tumor specific accumulation of ONPs was achieved by systemic delivery (FIG. 22E) and the IC₅₀ dose of Luc silencing was estimated around 2 mg/kg (FIG. 26A). Finally, the immune response of ONPs was monitored by measuring the IFN-α levels in blood samples 6 h after injection. We observed no significant increase in IFN-α compared to untreated mice (FIG. 26B).

The work described in this Example demonstrates that nucleic acid-based nanoparticle delivery systems, and particularly DNA tetrahedron nanoparticles formed from self-assembly of cross-hybridizing oligonucleotides, achieves targeted, in vivo siRNA delivery of siRNAs. Overhang design on nucleic acid strands in the nanoparticles allows specific hybridization of complementary siRNA sequences, providing control over spatial orientation of siRNA as well as the location and density of cancer targeting ligands. Particular advantages of these particles are the precise control over particle size, ligand orientation and density afforded by these particles. Those of ordinary skill, reading the present Example in light of the disclosure herein, will readily appreciate that described ONPs can be utilized to deliver any of a number of payload moieties, that can hybridize with single stranded sequences within or extending from the ONPs. The present Example thus establishes that ONP particles can provide a robust platform for the intracellular delivery of payloads. The present Example further establishes that particular ONP structures are surprisingly superior, when compared with other ONP structures, at achieving payload delivery. For example, particularly effective ONP structures, as defined herein include:

1. Those with folate targeting moieties;

2. Those with at least three targeting moieties, particularly at least three folate targeting moieties. In some embodiments, ONPs with precisely three targeting moieties show unique effectiveness;

3. Those with multiple targeting moieties on a single plane or face of the ONP particle, particularly at least three targeting moieties on a single plane or face, and specifically at least three folate moieties.

### Methods Summary

Preparation of Self-Assembled DNA/siRNA Nanoparticles (ONPs)

Six single strand DNAs from IDT (Chicago, Ill.) and 6 double strand siRNAs from Alnylam (Cambridge, Mass.) were annealed to prepare ONPs. Each DNA strand (final conc. each strand 8.33 μM) was mixed in equal molar ratio in annealing buffer containing Mg²⁺ (5 mM) and a 6 fold molar excess of siRNA strands were added to the solution. For annealing, sample solution was heated to 90° C. for 2 min and rapidly cooled to 4° C. to generate ONPs (8.33 μM). For folic acid conjugated ONPs, folic acid conjugated Luc or GFP siRNA were gifted from Alnylam and used without further purification. All siRNA was chemically modified during synthesis preparation to avoid immune response activation.⁹

In Vitro Testing of ONPs

Hela cells, which were modified to stably express both firefly and Renilla luciferase, were utilized for in vitro screening of ligands conjugated on ONPs. ONPs with different ligands were applied to 1.5×10⁴ Hela cells in serum containing medium. Firefly luciferase silencing was assessed 24 h post-transfection using a Dual-Glo Luciferase Assay kit (Promega, Madison, Wis.). Transfections were performed in quadruplicate. Lipofectamine RNAiMax (Invitrogen) was used as a positive control. For folate receptor targeted GFP gene silencing, GFP expressing KB cells were used. The level of GFP silencing was evaluated after 24 h post-transfection using a BD FACSCalibur (Franklin Lakes, N.J.).

siRNA Delivery in Mice Using ONPs

All animal experiments were conducted using institutionally approved protocols. Luc-KB tumor bearing female BALB/c nude mice (Charles River Laboratories, Wilmington, Mass.) received tail vein or intra-tumoral injections of either PBS (negative control) or ONPs containing anti-Luc siRNA diluted in PBS (n=7 per each group). Two days post-injection, bioluminescence intensity (BLI) in KB tumors was measured by a IVIS Lumina imaging system (Hopkinton, Mass.).

DNA Strands and Sticky-Overhang Sequence Design

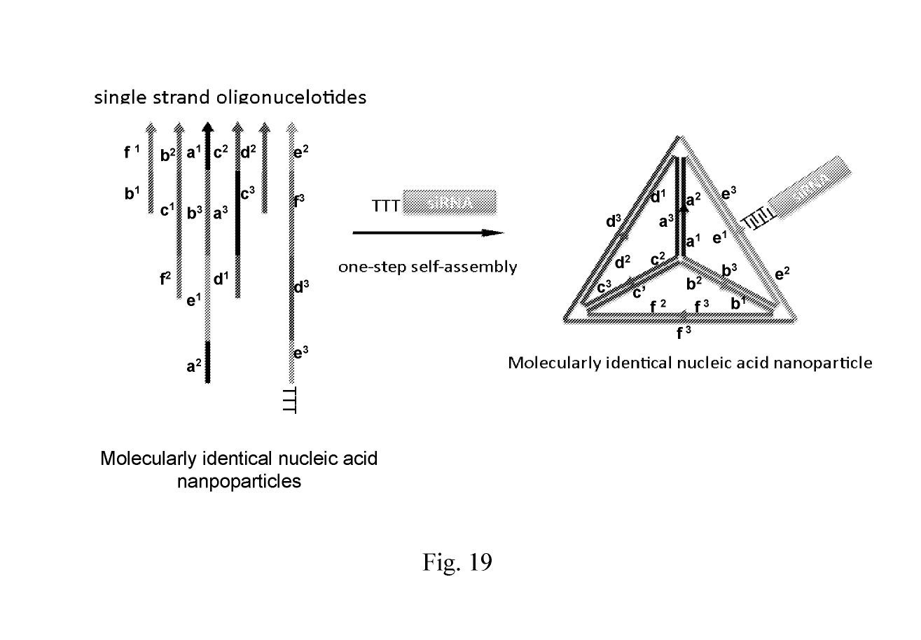

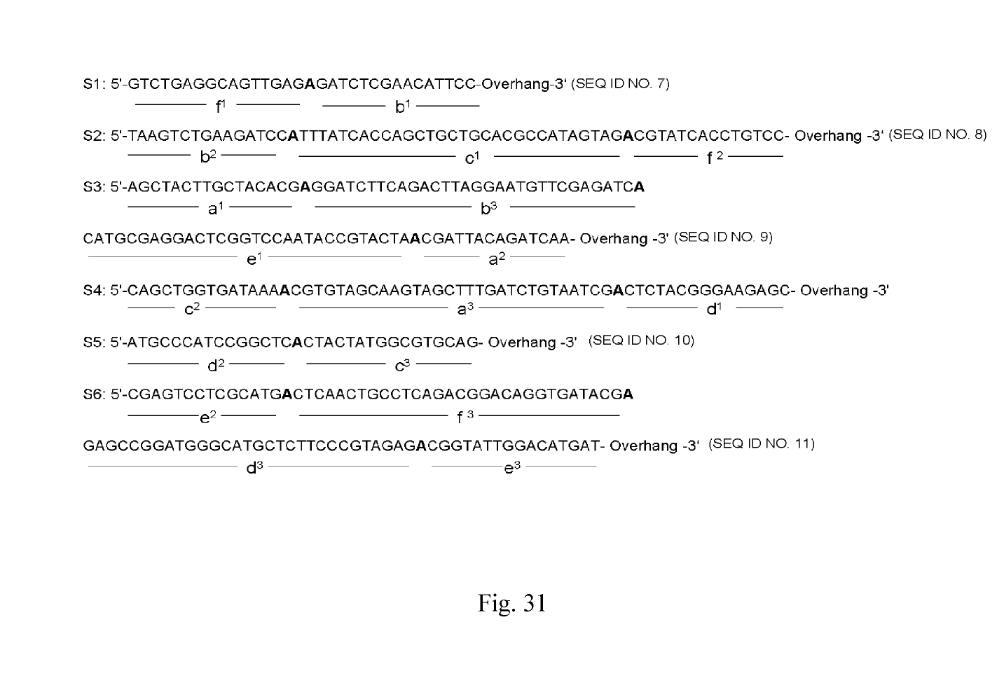

Sequence design is originally from Goodman et al. and modified to have 30 bp for each side along with a sticky-overhang for siRNA hybridization (FIG. 31). Subsequences corresponding to the edges of the tetrahedron are identified by the numeric code, consistent with that used in FIG. 19. Each DNA strand has an additional sticky-overhang sequences on the 3′ end (OH for all other experiment, except for ligand density study for FIG. 22).

Sticky-Overhang (OH) Design

### General Sticky-Overhang Sequence for In Vivo Study

(SEQ ID NO. 12)
OH: 5′-TTT TTT TTT TTT TTT TTT TTT-3′

Six Different Sticky-Overhang Sequences for Ligand Density Study

(SEQ ID NO. 13)
OH1: 5′-ATC GTA CGA TCA TAG ATC AAT-3′
(SEQ ID NO. 14)
OH2: 5′-TAC AGT CGT ATT GCA TTC CGA-3′
(SEQ ID NO. 15)
OH3: 5′-ATT CTA GAC GTT ACT TAA CAT-3′
(SEQ ID NO. 16)
OH4: 5′-TAA CTA TAG CTA CAA GCT TTC-3′
(SEQ ID NO. 17)
OH5: 5′-CCA TAC CGC CAT TTC CAA CTA-3′
(SEQ ID NO. 18)
OH6: 5′-AAG CAC ATG CGA TGT TTA ACT-3′

siRNA Sequences for Gene Silencing

anti-Luc siRNA
(SEQ ID NO. 19)
sense: 5′-Q37-AAcGcuGGaGuuAAucAAdTdT-L104-3′
(SEQ ID NO. 20)
antisense: 5′-UUGAUuAACGCCcAGCGUUdTdTOH^{c}-3′
anti-GFP siRNA
(SEQ ID NO. 21)
sense: 5′-Q37-AcAuGAAGcAGcACGACuUdTdT-L104-3′
(SEQ ID NO. 22)
antisense: 5′-AAGUCGUGCUGCUUCAUGUdTdTOH^{c}-3′

nomenclature: lower case letters=2′Omethyl modified RNA base

Q37 = Cy5
L104 = Floate
(SEQ ID NO. 23)
OH^{c} = 5′-AAA AAA AAA AAA AAA AAA AAA-3′

Self-Assembly of Tetrahedron DNA/siRNA Nanoparticles (ONPs)

Stoichiometric mixtures of component oligonucleotides (single strand DNA and double strand siRNA) were combined in TM buffer (10 mM Tris, 5 mM MgCl2), heated to 95° C. for 2 minutes, then rapidly cooled to 4° C. using a PCR machine (Eppendorf Mastercycler Personal). Particle size of ONPs was acquired in PBS using a ZETAPals analyzer (Brookhaven Instruments, Holtsville, N.Y.). All DNA strands were supplied by Integrated DNA Technologies and PAGE gel purified prior to the use. All siRNA strands were gifted from Alnylam Pharmaceutics.

Quantification

To quantify self-assembly yields, tetrahedron DNA nanoparticles with/without siRNA hybridization were run on a PAGE gel (5% TBE Ready Gels, Bio-Rad) with 1×TBE buffer and stained with SYBR Gold (Invitrogen). Band intensities were quantified with Image Lab software (Bio-Rad). Yields were estimated to be >95% for tetrahedron formation from 20 μM stock component oligonucleotides and >98% for siRNA hybridization, respectively.

AFM Imaging

10 μl of ONPs, diluted to 10 nM in TM buffer supplemented with 10 mM NiCl₂, were incubated for 15 minutes on freshly cleaved mica. After 15 minutes, buffer was added to a total volume of 200 μl and the sample was scanned in an MFD-3D atomic force microscope (Asylum Research, USA) operated in AC mode. Cantilever type: For general scanning, BL-AC40TS (Olympus Inc., Japan). For the high-resolution, SNL-10 (Veeco Inc., USA) with super-sharp tips of 2-3 nm radius were used.

Automated Confocal Microscopy

GFP expressing KB cells were seeded at 2.0×10⁴ cells/well in black 96-well plates with clear bottom (Corning, N.Y.). Cells were incubated with various concentrations (3.1-100 nM) of ONPs containing Cy5 fluorophore for 24 h at 37° C. Cells were washed with PBS then counter-stained in PBS-containing Hoescht (2 μg/ml) for nuclei identification. Stained live cell imaging was performed with an automated spinning disk confocal microscope (OPERA; Perkin Elmer, Shelton, Conn.) with a ×40 objective. The same defined pattern of 20 fields from each well was acquired to eliminate bias and provide a statistically significant number of cells for analysis. After identification of cell location and perimeter, siRNA content per each cell was calculated as well as the intensity of endogenous GFP using Acapella software. Data presented are an average of intracellular intensity from 20 different fields.

Preparation of Luc-KB Tumor Bearing Mice

All animal care and experimental procedures were conducted using institutionally approved protocols. For in vivo optical imaging, a xenograft tumor model was generated by subcutaneous injection of firefly Luc expressing KB (2×10⁶ cells/mouse) into a flank region of female nude mice (BALB/c). Tumor bearing mice were used when the diameter of tumors reached 6-8 mm.

In Vivo FMT-CT

CT and FMT imaging was done while anesthetized mice were restrained in a dedicated multi-modal imaging cassette (dimensions 50×30×280 mm; VisEn Medical). The nontransparent body of the cassette has an adjustable height to accommodate mice of different sizes and holds two transparent acrylic windows that allow laser excitation and photon emission in trans-illumination geometry during FMT imaging. During imaging, mice were anesthetized (isoflurane 1.5%, O2 2 L/min). An isoflurane delivery system is integrated into the multi-modal imaging cassette. 3 nmol of Cy5-labeled ONPs were administrated via tail-vein injection. We acquired 30 frontal slices of 0.5 mm thickness in the z direction, with an in-plane resolution of 1×1 mm. Time course images were taken every 5 min for 2 h, and then images were taken at 4, 6, 8, and 24 h post-injection. After image acquisition, datasets were post-processed using a normalized Born forward equation to calculate fluorochrome concentration, expressed in nM fluorescence per voxel, as described previously.^{S2}

mRNA Assay for Tumor Implants

The cellular level of firefly Luc mRNA was analyzed by a reverse transcriptase-polymerase chain reaction (RT-PCR). The tumor implants were collected from mice 2 days post-injection of ONPs. Tissue samples were homogenized and total RNA was isolated using Trizol reagent (Invitrogen, Carlsbad, Calif.) according to the manufacturer's protocol. Quantitative RT-PCR was performed with the purified total RNA (1 μg) using SuperScript™ III One-Step RT-PCR system with Platinum Taq DNA polymerase (Invitrogen, Carlsbad, Calif.). The real time PCR was performed according to our previous study.3 The PCR primers to detect firefly Luc and GAPDH were obtained from IDT (Chicago, Ill.).

Luc Primers Used:

(SEQ ID NO. 24)
Forward: TCC AAC ACC CCA ACA TCT TC
(SEQ ID NO. 25)
Reverse: GTC TTT CCG TGC TCC AAA AC
(SEQ ID NO. 26)
Probe: /56-FAM/AGA CCT GCG ACA CCT GCG T/36-TAMSp/

The PCR products were also analyzed by 2% agarose gel electrophoresis and visualized by SYBR gold (Invitrogen) staining.

IFN-α ELISA for Early Immune Response

To evaluate early immune responses in vivo, plasma IFN-α levels were determined using female C57BL/6 mice (Charles River Laboratory, Wilmington, Mass.). All mice were 7 weeks old and 20-25 g in body weight at the time of injection. A group of three mice was intravenously injected with siRNAs/Lipofectamine polyplexes and ONPs (2.5 mg/kg dose) in a 200 μl injection volume. Control mice were injected with PBS solution. After 6 h incubation, blood samples were collected from the mice and plasma IFN-α levels were analyzed by an ELISA kit (PBL Biomedical, Piscataway, N.J.).

### REFERENCES

1. Elbashir, S. M. et al. Duplexes of 21-nucleotide RNAs mediate RNA interference in cultured mammalian cells. Nature 411, 494-498 (2001).
2. Bumcrot, D., Manoharan, M., Koteliansky, V. and Sah, D. W. Y. RNAi therapeutics: a potential new class of pharmaceutical drugs. Nature Chem. Biol. 2, 711-719 (2006).
3. Whitehead, K. A., Langer, R., and Anderson, D. G. Knocking down barriers: advances in siRNA delivery. Nat. Rev. Drug Discov. 8, 129-138 (2009).
4. Oh, Y. K., and Park, T. G. siRNA delivery systems for cancer treatment, Adv. Drug Deliv. Rev. 61, 850-862 (2009).
5. Allen, T. M., and Cullis, P. R. Drug delivery systems: entering the mainstream. Science 303, 1818-1822 (2004).
6. Thomas, M. et al. Full deacylation of polyethylenimine dramatically boosts its gene delivery efficiency and specificity to mouse lung. Proc. Natl Acad. Sci. USA 102, 5679-5684 (2005).
7. Lv, H., Zhang, S., Wang, B., Cui, S., and Yan, J. Toxicity of cationic lipids and cationic polymers in gene delivery, J. Control. Release 114, 100-109 (2006).
8. Goodman, R. P. et al. Rapid chiral assembly of rigid DNA building blocks for molecular nanofabrication, Science 310, 1661-1664 (2005).
9. Gaglione, M., and Messere, A. Recent progress in chemically modified siRNAs, Mini Rev. Med. Chem. 10, 578-595 (2010).
10. Guo, P. et al. Engineering RNA for targeted siRNA delivery and medical application. Adv. Drug Deliv. Rev. 62, 650-666 (2010).
11. Davis, M. R. et al. Evidence of RNAi in humans from systemically administered siRNA via targeted nanoparticles. Nature 464, 1067-1071 (2010).
12. Song, E. et al. Antibody mediated in vivo delivery of small interfering RNAs via cell-surface receptors. Nature Biotech. 23, 709-717 (2005).
13. Li, S. D., Chen, Y. C., Hackett, M. J., and Huang, L. Tumor-targeted delivery of siRNA by self-assembled nanoparticles, Mol. Ther. 16,163-169 (2008).
14. Tarapore, P., Shu, Y., Guo, P., and Ho, S. M. Application of Phi29 motor pRNA for targeted therapeutic delivery of siRNA silencing metallothionein-IIA and surviving in ovarian cancers. Mol. Ther. 19, 386-394 (2011).
15. Manz, B. N. et al. T-cell triggering thresholds are modulated by the number of antigen within individual T-cell receptor clusters. Proc. Natl Acad. Sci. USA 108, 9089-9094 (2011).
16. Nahrendorf, M. et al. Hybrid PET-optical imaging using targeted probes. Proc. Natl. Acad. Sci. USA 107, 7910-7915 (2010).
17. Nahrendorf, M. et al. Hybrid in vivo FMT-CT imaging of protease activity in atherosclerosis with customized nanosensors. Arterioscler. Thromb. Vasc. Biol. 29, 1444-1451 (2009).
18. Soutschek, J. et al. Therapeutic silencing of an endogenous gene by systemic administration of modified siRNAs. Nature 432, 173-178 (2004).
51. Goodman, R. P. et al. Rapid chiral assembly of rigid DNA building blocks for molecular nanofabrication, Science 310, 1661-1664 (2005).
S2. Nahrendorf, M. et al. Hybrid PET-optical imaging using targeted probes, Proc. Natl. Acad. Sci. USA 107, 7910-7915 (2010).
S3. Love, K. T. et al. Lipid-like materials for low-dose, in vivo gene silencing, Proc. Natl. Acad. Sci. USA 107, 1864-1869 (2010).

### Example 2

### Design of siRNA Payload Structures

The present Example describes analyses that demonstrate different potencies of differently structured siRNA payload agents for use in accordance with the present invention. In particular, the present Example demonstrates surprising superiority of double-stranded siRNA payload agents having 3′ overhangs (that can act as ligand sequence in accordance with the present invention) as compared with double-stranded siRNA payload agents having 5′ overhangs. Double-stranded siRNA payload agents without overhangs were also effective, though less potent than the 3 ‘-overhang-containing agents. Agents with dual overhangs (3’ and 5′) were not effective.

Accordingly, in some embodiments, the present invention delivers double stranded siRNA payloads lacking an overhang or containing a 3′ overhang. In some particular embodiments, the present invention delivers double stranded siRNA payloads with a 3′ overhang. In some embodiments, the present invention utilizes double-stranded siRNA payloads that lack an overhang but are biologically or chemically linked (e.g., covalently linked, for example by “branching” from the siRNA backbone) to a single-stranded oligonucleotide comprising a ligand hybridization element.

In some embodiments, nanoparticle delivery structures as described herein for delivering such siRNA payloads comprise ligand hybridization elements complementary to an siRNA overhang. In some such embodiments, the ligand hybridization element is or comprises a 5′ single stranded portion.

Materials

GFP-siRNAs with different sticky overhang location were obtained from IDT.

Sequences of these siRNAs were:

(SEQ ID NO. 27)
Sense GFP: 5′-AAC UUC AGG GUC AGC UUG CdTdT-3′
(SEQ ID NO. 28)
AS GFP: 5′-GCA AGC UGA CCC UGA AGU UdTdT-3′

5′ Overhang Sense GFP:

(SEQ ID NO. 29)
5′-dAdAdA dAdAdA dAdAdA dAdAdA dAdAdA dAdAdA dAdA
AAC UUC AGG GUC AGC UUG CdTdT-3′

3′ Overhang AS GFP:

(SEQ ID NO. 30)
5′-GCA AGC UGA CCC UGA AGU UdTdT dAdAdA dAdAdA
dAdAdA dAdAdA dAdAdA dAdAdA dAdA-3′

For delivering agent, commercially available Lipofectamine (LF) RNAi Max (Invitrogen) was used for in vitro assays.

Methods

1. Hybridization of Double Stranded siRNA

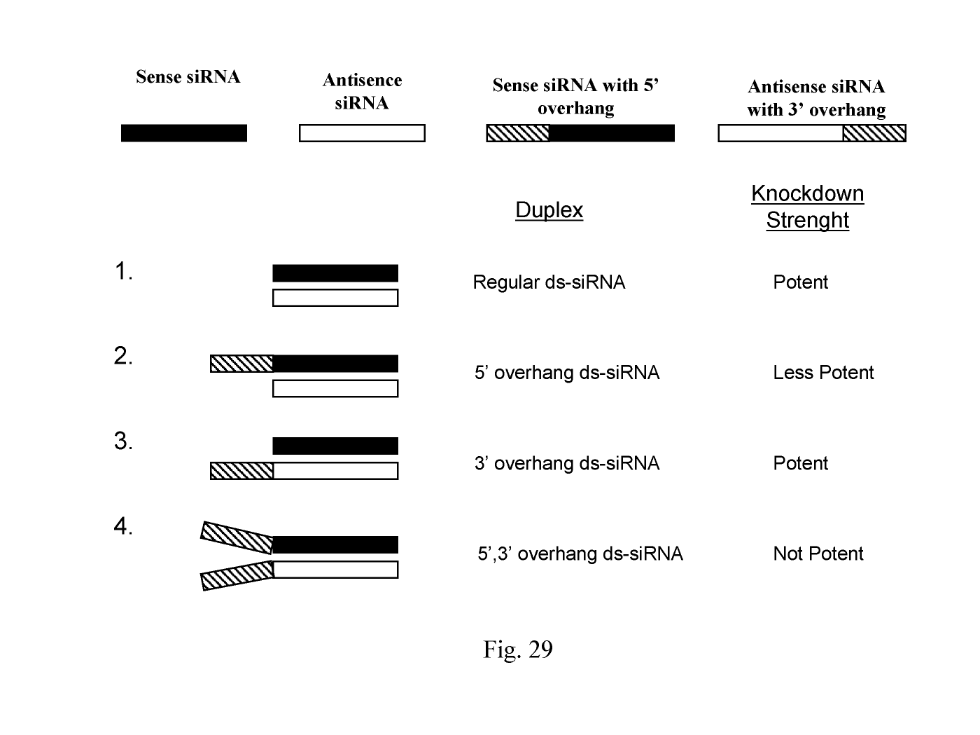

For the preparation of ds-siRNA, sense and antisense strand in PBS were mixed together in equal molar ratio. Hybridization was confirmed by PAGE (10% native PAGE) assay. There were four different samples generated using sticky overhang containing siRNA, including one control which does not have overhang on its structure (FIG. 29).

2. Transfection of GFP Expressing KB Cells

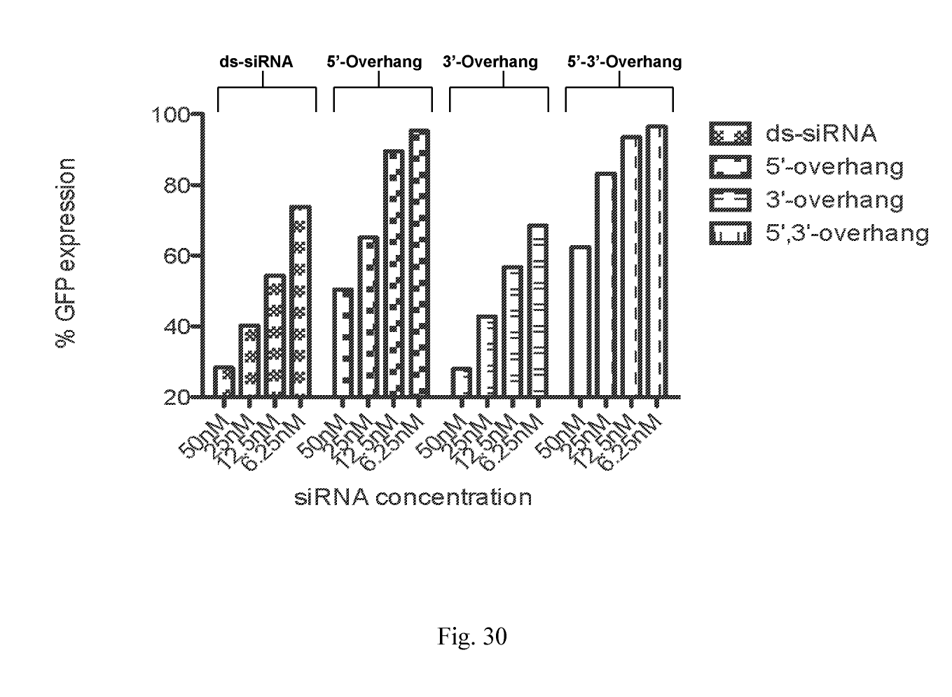

For the gene silencing experiment in vitro, stable GFP expressing KB cells were utilized. For the transfection, 2×10⁵ cells were plated in 6 well culture plate and incubated for overnight. To this, various concentrations of ds-siRNA were treated (50, 25, 12.5, and 6.25 nM). Briefly, for 50 nM concentration, 100 pmol (˜1.3 ug) of ds-siRNA were complexed with 2.5 ul of LF solution and incubated for 15 min, then applied to the cells for 24 hr. Cells were treated with trypsin and collected in 1 ml PBS containing 1% FBS. Fluorescence level of transfected cells was determined by FACS. KB cells without treatment was used as negative control and the fluorescence level was normalized to the control (FIG. 30).

### Example 3

### Design of Ligand Hybridization Elements

The present Example describes certain insights provided by the present disclosure with respect to design of ligand hybridization elements in nanoparticle delivery systems. In particular, in accordance with the present invention, length of such ligand hybridization elements (and/or of their complementary ligand sequences) can be varied depending on the application. However, in certain embodiments, a minimum length is utilized that gives sufficient stability under body temperature (37° C.).

In some embodiments, natural DNA bases are utilized; in some such embodiments, minimum overhang length is determined by calculating the melting temperature of double stranded DNA for overhang sequences and the melting temperature should be higher than 37° C. Typical overhang length for natural DNA bases to have melting temperature above body temperature is in the range of 15 bp˜30 bp depending on the sequence design.

In some embodiments, one or more non-natural DNA bases are utilized; those of ordinary skill will appreciate strategies for calculating melting temperature, and therefore for determining appropriate length and/or content of ligand hybridization elements and/or ligand sequences including such non-natural DNA bases, for use as described herein.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

We claim:

1. A delivery system comprising:
a nanoparticle comprised of a plurality of DNA oligonucleotides, each of which has a structure comprising:
a first portion that is complementary to a first portion of another oligonucleotide in the plurality such that the plurality of oligonucleotides self-assembles to form the nanoparticle, which is a three-dimensional tetrahedral structure comprising a plurality of faces defined by substantially double stranded arms,
wherein at least some oligonucleotides within the plurality have a structure that further comprises:
a second portion that does not hybridize with another oligonucleotide in the plurality, so that such second portions comprises a ligand hybridization element, available for hybridization to a ligand associated with a payload moiety,
a folate targeting moiety; and
an siRNA payload moiety;
wherein the delivery system is configured and arranged such that the payload moiety is liberated from the delivery system and deposited within a cell.

2. The delivery system of claim 1, wherein the ligand hybridization element comprises a single stranded overhang.

3. The delivery system of claim 1, wherein the ligand hybridization element comprises a single stranded 5′ overhang.

4. (canceled)

5. The delivery system of claim 1, wherein the nanoparticle comprises two or more siRNA payload moieties.

6. The delivery system of claim 1, wherein the siRNA payload moiety has a 3′ overhang.

7. The delivery system of claim 6, wherein at least one siRNA payload moiety is hybridized to the nanoparticle by way of its 3′ overhang.

8. (canceled)

9. The delivery system of claim 1, wherein the ligand and ligand hybridization element each have a sequence selected from the group consisting of polyA and polyT.

10. The delivery system of claim 1, wherein the delivery system comprises at least three targeting moieties, wherein the oligonucleotides within the plurality being designed and constructed such that, when the at least three targeting moieties hybridize to the ligand hybridization elements, the at least three targeting moieties are displayed on the same face of the nanoparticle.

11-24. (canceled)

25. The delivery system of claim 1, wherein at least one ligand hybridized element comprises a cleavage site.

26-54. (canceled)

55. A method of delivering a payload moiety to a cell comprising the steps of:
A) forming a nanoparticle with a three-dimensional tetrahedral structure from a plurality of self-assembling DNA oligonucleotides, each of which having a structure which comprises:
i) a first portion that is complementary to a first portion of another oligonucleotide in the plurality such that the plurality of oligonucleotides self-assembles to form the nanoparticle, which has a three-dimensional structure comprising a plurality of faces defined by substantially double stranded arms, wherein at least some oligonucleotides within the plurality have a structure that further comprises:
(a) a second portion that does not hybridize with another oligonucleotide in the plurality, so that such second portions comprises a ligand hybridization element, available for hybridization to a ligand associated with a payload moiety
ii) a folate targeting moiety; and
iii) an siRNA payload moiety;
B) administering the nanoparticle to a plurality of cells;
C) binding of the folate targeting moiety to a corresponding cellular target associated with a defined subset of cells within the plurality of cells;
D) internalizing of the nanoparticle by the subset of cells; and
E) releasing the siRNA payload from the ligand hybridization element, such that the siRNA payload moiety is release.

56. The method of claim 15, wherein the ligand hybridization element comprises a single stranded overhang.

57. The method of claim 15, wherein the ligand hybridization element comprises a single stranded 5′ overhang.

58. The method of claim 15, wherein the nanoparticle comprises two or more payload moieties.

59. The method of claim 15, wherein the siRNA payload moiety has a 3′ overhang.

60. The method of claim 19, wherein the siRNA payload moiety is hybridized to the nanoparticle by way of its 3′ overhang.

61. The method of claim 15, wherein the ligand and ligand hybridization element each have a sequence selected from the group consisting of polyA and polyT.

62. The method of claim 15, wherein the nanoparticle comprises at least three targeting moieties, wherein the oligonucleotides within the plurality being designed and constructed such that, when the at least three targeting moieties hybridize to the ligand hybridization elements, the at least three targeting moieties are displayed on the same face of the nanoparticle.

63. The method of claim 22, wherein the at least three targeting moieties are each a folate targeting moiety.

64. The method of claim 23, wherein the at least three targeting moieties target an intracellular marker.

65. The delivery system of claim 23, wherein the at least three targeting moieties target an extracellular marker.

66. The delivery system of claim 22, wherein the at least three targeting moieties target a cancer cell.
